SEQUENCE LISTING (64 sequences)

SEQ ID NO: 1 - DNA, 823 bp, artificial sequence
  synthetic sequence
ctagcctaccctccatcattgttataaaaaacttaggaaccaggtccacacagccgccagcccatcaacgcgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagttcgaactacagctcaacttacct

SEQ ID NO: 2 - DNA, 144 bp, artificial sequence
  synthetic sequence
ttcgaactacagctcaacttacctgccaaccccatgccagacgacccaactagtacaacctaaatccattataaaaaacttaggaaccaggtccacacagccgccagcccatcaaccatccactcccacgattggaggccggcc

SEQ ID NO: 3 - DNA, 7648 bp, artificial sequence
  synthetic sequence
ccatcacattgggatcctaagcttagaattccacgtggacaagttgtcgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattaatacgactcactatagggtcagtgcggccgcgacttcaagtcacgtgaattccaagcttcggatcccactgtggtgg

SEQ ID NO: 4 - DNA, 7591 bp, artificial sequence
  synthetic sequence
ttataaaaaacttaggaaccaggtccacacagccgccagcccatcaacgcgtacgatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgagagcggtaccccaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtcaagggcaccatcgataacgcggacctggccaaactggcctttaagcggtcatctaagtatgaccttgaatgcgctcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccatgctcccagccaccttgcacgccctgctgctacgagaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtcctaaaggcgaaactctgacggtgggattcactgacagtaggaagattagtcactcatgtacgcacccatttcaccacgaccctcctgtgataggtcgggagaaattccattcccgaccgcagcacggtaaagagctaccttgcagcacctacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccggaaaggaaaaattcacatcccgtttccgctggcaaatgtaacatgcagggtgcctaaagcaaggaaccccaccgtgacgtatggcaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcacgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtcccattcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcttacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccttacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgcccttcggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaatagctacagctcaacttacctgccaaccccatgccagacgacccaactagtacaacctaaatccattataaaaaacttaggaaccaggtccacacagccgccagcccatcaaccatccactcccacgattggaggccggccatgagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggtcaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggattatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtctaattcgaactacagctcaacttacctgccaaccccatgccagacgacccaactagtacaacctaaatccattataaaaaacttaggaaccaggtccacacagccgccagcccatcaaccatccactcccacgattggagcggccgcatggagaagaagcggagaggagcagacacaagcgtgggaatcgtgggcctgctgctgaccacagcaatggcagcagaggtgaccaggagaggaagcgcctactatatgtacctggacaggaatgatgccggcgaggccatctccttcccaaccacactgggcatgaacaagtgctacatccagatcatggacctgggccacatgtgcgatgccaccatgtcctatgagtgtccaatgctggacgagggcgtggagcccgacgatgtggattgctggtgtaataccacatctacatgggtggtgtacggcacctgtcaccacaagaagggagaggcccggcggagccggcgggccgtgacactgccttcccactctaccaggaagctgcagacacgcagccagacctggctggagtccagagagtataccaagcacctgatcagggtggagaactggatctttcgcaatccaggattcgcactggcagcagcagcaatcgcatggctgctgggaagctccaccagccagaaagtgatctacctggtcatgatcctgctgatcgctcctgcctattctatccggtgcatcggcgtgagcaatagagacttcgtggagggaatgtccggaggaacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcaggtgtccaacccagggagaggcatacctggacaagcagtccgatacacagtacgtgtgcaagcggaccctggtggacagaggctggggcaatggctgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactctccaagagccgaggccaccctgggaggatttggctctctgggcctggactgcgagcctagaacaggcctggacttctccgatctgtactatctgaccatgaacaataagcactggctggtgcacaaggagtggtttcacgacatcccactgccatggcacgcaggagcagatacaggaacaccacactggaacaataaggaggccctggtggagttcaaggatgcccacgccaagcggcagacagtggtggtgctgggcagccaggagggagcagtgcacaccgccctggcaggcgccctggaggcagagatggacggagctaagggcagactgtctagcggccacctgaagtgcaggctgaagatggataagctgcgcctgaagggcgtgtcctactctctgtgcacagccgccttcaccttcaccaagatccctgccgagacactgcacggcacagtgaccgtggaggtgcagtatgccggcacagacggaccctgtaaggtgcctgcccagatggccgtggatatgcagacactgacacctgtgggcaggctgatcaccgccaatccagtgatcacagagtctaccgagaacagcaagatgatgctggagctggacccaccatttggcgatagctatatcgtgatcggcgtgggcgagaagaagatcacacaccactggcaccgcagcggctccacaatcggcaaggcctttgaggcaaccgtgcgcggagcaaagagaatggccgtgctgggcgacaccgcatgggatttcggatctgtgggaggcgccctgaacagcctgggcaagggcatccaccagatcttcggcgccgcctttaagtccctgttcggcggcatgagctggttctcacagatcctgatcggcacactgctgatgtggctgggcctgaacaccaagaatggctctatcagcctgatgtgcctggccctgggaggcgtgctgatcttcctgtccaccgccgtgtctgcctgatgataatagttcgaactacagctcaacttacct

SEQ ID NO: 5 - DNA, 18967 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 6 - DNA, 19795 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 7 - DNA, 25717 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagttcgaactacagctcaacttacctgccaaccccatgccagacgacccaactagtacaacctaaatccattataaaaaacttaggaaccaggtccacacagccgccagcccatcaaccatccactcccacgattggaggccggccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgtagtaaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 8 - DNA, 24062 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 9 - DNA, 7648 bp, artificial sequence; identical to SEQ ID NO: 3
  synthetic sequence

SEQ ID NO: 10 - DNA, 7859 bp, artificial sequence
  synthetic sequence
gcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 11 - DNA, 144 bp, artificial sequence
  synthetic sequence
  misc_feature (1)..(6): n is a, c, g, or t
  misc_feature (137)..(144): n is a, c, g, or t
nnnnnnctacagctcaacttacctgccaaccccatgccagacgacccaactagtacaacctaaatccattataaaaaacttaggaaccaggtccacacagccgccagcccatcaaccatccactcccacgattggannnnnnnn

SEQ ID NO: 12 - DNA, 31676 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgagagcggtaccccaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtcaagggcaccatcgataacgcggacctggccaaactggcctttaagcggtcatctaagtatgaccttgaatgcgctcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccatgctcccagccaccttgcacgccctgctgctacgagaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtcctaaaggcgaaactctgacggtgggattcactgacagtaggaagattagtcactcatgtacgcacccatttcaccacgaccctcctgtgataggtcgggagaaattccattcccgaccgcagcacggtaaagagctaccttgcagcacctacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccggaaaggaaaaattcacatcccgtttccgctggcaaatgtaacatgcagggtgcctaaagcaaggaaccccaccgtgacgtatggcaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcacgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtcccattcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcttacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccttacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgcccttcggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaatagctacagctcaacttacctgccaaccccatgccagacgacccaactagtacaacctaaatccattataaaaaacttaggaaccaggtccacacagccgccagcccatcaaccatccactcccacgattggaggccggccatgagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggtcaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggattatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtctaattcgaactacagctcaacttacctgccaaccccatgccagacgacccaactagtacaacctaaatccattataaaaaacttaggaaccaggtccacacagccgccagcccatcaaccatccactcccacgattggagcggccgcatggagaagaagcggagaggagcagacacaagcgtgggaatcgtgggcctgctgctgaccacagcaatggcagcagaggtgaccaggagaggaagcgcctactatatgtacctggacaggaatgatgccggcgaggccatctccttcccaaccacactgggcatgaacaagtgctacatccagatcatggacctgggccacatgtgcgatgccaccatgtcctatgagtgtccaatgctggacgagggcgtggagcccgacgatgtggattgctggtgtaataccacatctacatgggtggtgtacggcacctgtcaccacaagaagggagaggcccggcggagccggcgggccgtgacactgccttcccactctaccaggaagctgcagacacgcagccagacctggctggagtccagagagtataccaagcacctgatcagggtggagaactggatctttcgcaatccaggattcgcactggcagcagcagcaatcgcatggctgctgggaagctccaccagccagaaagtgatctacctggtcatgatcctgctgatcgctcctgcctattctatccggtgcatcggcgtgagcaatagagacttcgtggagggaatgtccggaggaacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcaggtgtccaacccagggagaggcatacctggacaagcagtccgatacacagtacgtgtgcaagcggaccctggtggacagaggctggggcaatggctgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactctccaagagccgaggccaccctgggaggatttggctctctgggcctggactgcgagcctagaacaggcctggacttctccgatctgtactatctgaccatgaacaataagcactggctggtgcacaaggagtggtttcacgacatcccactgccatggcacgcaggagcagatacaggaacaccacactggaacaataaggaggccctggtggagttcaaggatgcccacgccaagcggcagacagtggtggtgctgggcagccaggagggagcagtgcacaccgccctggcaggcgccctggaggcagagatggacggagctaagggcagactgtctagcggccacctgaagtgcaggctgaagatggataagctgcgcctgaagggcgtgtcctactctctgtgcacagccgccttcaccttcaccaagatccctgccgagacactgcacggcacagtgaccgtggaggtgcagtatgccggcacagacggaccctgtaaggtgcctgcccagatggccgtggatatgcagacactgacacctgtgggcaggctgatcaccgccaatccagtgatcacagagtctaccgagaacagcaagatgatgctggagctggacccaccatttggcgatagctatatcgtgatcggcgtgggcgagaagaagatcacacaccactggcaccgcagcggctccacaatcggcaaggcctttgaggcaaccgtgcgcggagcaaagagaatggccgtgctgggcgacaccgcatgggatttcggatctgtgggaggcgccctgaacagcctgggcaagggcatccaccagatcttcggcgccgcctttaagtccctgttcggcggcatgagctggttctcacagatcctgatcggcacactgctgatgtggctgggcctgaacaccaagaatggctctatcagcctgatgtgcctggccctgggaggcgtgctgatcttcctgtccaccgccgtgtctgcctgatgaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 13 - protein, 1248 aa, artificial sequence
  synthetic sequence
MEFIPTQTFYNRRYQPRPWTPRPTIQVIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLAIPVMCLLANTTFPCSQPPCTPCCYEKEPEETLRMLEDNVMRPGYYQLLQASLTCSPHRQRRSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKAATYQEAAIYLWNEQQPLFWLQALIPLAALIVLCNCLRLLPCCCKTLAFLAVMSVGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 14 - protein, 459 aa, artificial sequence
  synthetic sequence
MRVVFVVLLLLVAPAYSFNCLGMSNRDFLEGVSGATWVDLVLEGDSCVTIMSKDKPTIDVKMMNMEAVNLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAV

SEQ ID NO: 15 - protein, 696 aa, artificial sequence
  synthetic sequence
MEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 16 - DNA, 58 bp, artificial sequence
  synthetic sequence
ttataaaaaacttaggaaccaggtccacacagccgccagcccatcaacgcgtacgatg

SEQ ID NO: 17 - DNA, 84 bp, artificial sequence
  synthetic sequence
taataggcgcgcagcgcttagacgtctcgcgatcgatactagtacaacctaaatccattataaaaaacttaggagcaaagtgat

SEQ ID NO: 18 - DNA, 8 bp, artificial sequence
  synthetic sequence
gcggccgc

SEQ ID NO: 19 - DNA, 78 bp, artificial sequence
  synthetic sequence
tataaaaaacttaggaaccaggtccacacagccgccagcccatcaaccatccactcccacgattggaggccggccatg

SEQ ID NO: 20 - DNA, 78 bp, artificial sequence
  synthetic sequence
tataaaaaacttaggaaccaggtccacacagccgccagcccatcaaccatccactcccacgattggagcggccgcatg

SEQ ID NO: 21 - DNA, 31 bp, artificial sequence
  synthetic sequence
cattactcctacgcgagcaattaacgaatcc

SEQ ID NO: 22 - DNA, 31 bp, artificial sequence
  synthetic sequence
ggattcgttaattgctgccgtaggagtaatg

SEQ ID NO: 23 - DNA, 31 bp, artificial sequence
  synthetic sequence
acccaggttaggcgcactgtcaataactatg

SEQ ID NO: 24 - DNA, 31 bp, artificial sequence
  synthetic sequence
catagttattgacagtgcgcctaacctgggt

SEQ ID NO: 25 - DNA, 32 bp, artificial sequence
  synthetic sequence
tgatttaaattcgtttagtgaaccgtcagatc

SEQ ID NO: 26 - DNA, 32 bp, artificial sequence
  synthetic sequence
ttgcggccgcagtactctagccttaagagctg

SEQ ID NO: 27 - DNA, 32 bp, artificial sequence
  synthetic sequence
ttacgtacgatggagttcatcccaacccaaac

SEQ ID NO: 28 - DNA, 96 bp, artificial sequence
  synthetic sequence
ataatggatttaggttgtactagttgggtcgactggcatggggttggcaggtaagttgagctgtagttcgaactattagtgcctgctgaacgacac

SEQ ID NO: 29 - DNA, 106 bp, artificial sequence
  synthetic sequence
aacctaaatccattataaaaaacttaggaaccaggtccacacagccgccagcccatcaaccatccactcccacgattggacctgcaggatgagagttgtgtttgtc

SEQ ID NO: 30 - DNA, 90 bp, artificial sequence
  synthetic sequence
ataatggatttaggttgtactagttgggtcgactggcatggggttggcaggtaagttgagctgtagttcgaattagacagccttcccaac

SEQ ID NO: 31 - DNA, 107 bp, artificial sequence
  synthetic sequence
aacctaaatccattataaaaaacttaggaaccaggtccacacagccgccagcccatcaaccatccactcccacgattggaggccggccatggagaagaagcggagag

SEQ ID NO: 32 - DNA, 27 bp, artificial sequence
  synthetic sequence
tgagcgcgcttagacagccttcccaac

SEQ ID NO: 33 - DNA, 25 bp, artificial sequence
  synthetic sequence
ttagcgcgctcatcaggcagacacg

SEQ ID NO: 34 - DNA, 29 bp, artificial sequence
  synthetic sequence
attcctgcaggatgagagttgtgtttgtc

SEQ ID NO: 35 - DNA, 30 bp, artificial sequence
  synthetic sequence
ttacctgcaggatggagaagaagcggagag

SEQ ID NO: 36 - DNA, 28 bp, artificial sequence
  synthetic sequence
ttacgtacgatggagaagaagcggagag

SEQ ID NO: 37 - DNA, 97 bp, artificial sequence
  synthetic sequence
ataatggatttaggttgtactagttgggtcgactggcatggggttggcaggtaagttgagctgtagttcgaatcatcaggcagacacggcggtggac

SEQ ID NO: 38 - DNA, 111 bp, artificial sequence
  synthetic sequence
aacctaaatccattataaaaaacttaggaaccaggtccacacagccgccagcccatcaaccatccactcccacgattggacctgcaggatggagttcatcccaacccaaac

SEQ ID NO: 39 - DNA, 31 bp, artificial sequence
  synthetic sequence
ttagcgcgcctattagtgcctgctgaacgac

SEQ ID NO: 40 - DNA, 36 bp, artificial sequence
  synthetic sequence
cttaggattcaagatcctattatcagggacaagagc

SEQ ID NO: 41 - DNA, 36 bp, artificial sequence
  synthetic sequence
cttaggaaccaggtccacacagccgccagcccatca

SEQ ID NO: 42 - DNA, 35 bp, artificial sequence
  synthetic sequence
cttaggagcaaagtgattgcctcccaaggtccaca

SEQ ID NO: 43 - DNA, 34 bp, artificial sequence
  synthetic sequence
cttagggccaaggaacatacacacccaacagaac

SEQ ID NO: 44 - DNA, 23 bp, artificial sequence
  synthetic sequence
cttagggtgcaagatcatccaca

SEQ ID NO: 45 - DNA, 25 bp, artificial sequence
  synthetic sequence
cgtagggtccaagtggttccccgtt

SEQ ID NO: 46 - DNA, 33 bp, artificial sequence
  synthetic sequence
ccgcctaccatccatcattgttataaaaaactt

SEQ ID NO: 47 - DNA, 33 bp, artificial sequence
  synthetic sequence
actagtacaacctaaatccattataaaaaactt

SEQ ID NO: 48 - DNA, 33 bp, artificial sequence
  synthetic sequence
gatcctctctccccggcaaactaaacaaaactt

SEQ ID NO: 49 - DNA, 20 bp, artificial sequence
  synthetic sequence
tcggtagttaatcaaaactt

SEQ ID NO: 50 - DNA, 33 bp, artificial sequence
  synthetic sequence
gtgtgaaatagacatcagaattaagaaaaacgt

SEQ ID NO: 51 - DNA, 33 bp, artificial sequence
  synthetic sequence
taggcattatttgcaatatattaaagaaaactt

SEQ ID NO: 52 - DNA, 116 bp, artificial sequence
  synthetic sequence
  misc_feature (49)..(49): n is a, c, g, or t
cttaggattcaagatcctattatcagggacaagagcacgcgtacgatgntaggcgcgcagcgcttagacgtctcgcgatcgatccgcctaccatccatcattgttataaaaaactt

SEQ ID NO: 53 - DNA, 116 bp, artificial sequence
  synthetic sequence
  misc_feature (49)..(49): n is a, c, g, or t
cttaggaaccaggtccacacagccgccagcccatcaacgcgtacgatgntaggcgcgcagcgcttagacgtctcgcgatcgatactagtacaacctaaatccattataaaaaactt

SEQ ID NO: 54 - DNA, 115 bp, artificial sequence
  synthetic sequence
  misc_feature (48)..(48): n is a, c, g, or t
cttaggagcaaagtgattgcctcccaaggtccacaacgcgtacgatgntaggcgcgcagcgcttagacgtctcgcgatcgatgatcctctctccccggcaaactaaacaaaactt

SEQ ID NO: 55 - DNA, 101 bp, artificial sequence
  synthetic sequence
  misc_feature (47)..(47): n is a, c, g, or t
cttagggccaaggaacatacacacccaacagaacacgcgtacgatgntaggcgcgcagcgcttagacgtctcgcgatcgattcggtagttaatcaaaactt

SEQ ID NO: 56 - DNA, 103 bp, artificial sequence
  synthetic sequence
  misc_feature (36)..(36): n is a, c, g, or t
cttagggtgcaagatcatccacaacgcgtacgatgntaggcgcgcagcgcttagacgtctcgcgatcgatgtgtgaaatagacatcagaattaagaaaaacgt

SEQ ID NO: 57 - DNA, 105 bp, artificial sequence
  synthetic sequence
  misc_feature (38)..(38): n is a, c, g, or t
cgtagggtccaagtggttccccgttacgcgtacgatgntaggcgcgcagcgcttagacgtctcgcgatcgattaggcattatttgcaatatattaaagaaaactt

SEQ ID NO: 58 - DNA, 15894 bp, artificial sequence
  synthetic sequence
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggacaaatacttttcacatgatgatccaattagtagtgatcaatccaggttcggatggttcgagaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgaggaccagaacaacatccgcctaccctccatcattgttataaaaaacttaggaaccaggtccacacagccgccagcccatcaaccatccactcccacgattggagccgatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattatgtttatgatcacagcggtgaagcggttaagggaatccaagatgctgactctatcatggttcaatcaggccttgatggtgatagcaccctatcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtatgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagccaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggtcgatcgctccgatacaaccgaccacctacagtgatggcaggctggtgccccaggtcagagtcatagatcctggtctaggcgacaggaaggatgaatgctttatgtacatgtctctgctgggggttgttgaggacagcgatcccctagggcctccaatcgggcgagcatttgggtccctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccactgaccaagcgagaggccagccagcagccgacggcaagcacgaacaccaggcggccccagcacagaacagccctgatacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacctaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgataactgcccggtagtcgaggtgaacggcgtgaccatccaagtcgggagcaggaggtatccagacgctgtgtacttgcacagaattgacctcggtcctcccatattattggagaggttggacgtagggacaaatctggggaatgcaattgctaagttggaggatgccaaggaattgttggagtcatcggaccagatattgaggagtatgaaaggtttatcgagcacttgcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcaccgggaagattctatcacaattccctatcagggatcagggaaaggtgtcagcttccagctcgtcaagctaggtgtctggaaatccccaaccgacatgcaatcctgggtcaccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccaccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaactaatctcatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcctgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatacaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccaggggcctgcccaccaattcgaggtctaagaccggtagagaaatgtgcagttctaaccgaccatatcaaggcagaggctaggttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctaacaaagatactatagagaagctagaggaattggcagccatcttatcgatggctctgctcctgggcaaaataggatcaatactggtgattaagcttatgcctttcagcggggattttgttcagggatttataagttatgtagggtcttattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccgaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattgattgaactccggaaccctaatcctgccctaggtggttaggcattatttgcaatatattaaagaaaactttgaaaatacgaagtttctattcccagctttgtctggt

SEQ ID NO: 59 - DNA, 15894 bp, artificial sequence
  synthetic sequence
accaaacaaagttgggtaaggatagttcaatcaatgatcattttctagtgcacttaggattcaagatcctattatcagggacaagagcaggattaaggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgagaacaaggaaatctcagatattgaagtgcaagaccctgagggattcaacatgattctgggtaccatcctagctcaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccctccatcattgttataaaaaacttaggaaccaggtccacacagccgccagcccatcaaccatccactcccacgattggagccgatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattatgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcctccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggttgatcgctccgatacaacccaccacctacagtgatggcaggctggtgccccaggtcagagtcatagatcctggtctaggcgacaggaaggatgaatgctttatgtacatgtttctgctgggggttgttgaggacagcgatcccctagggcctccaatcgggcgagcatttgggtccctgcccttaggtgttggcaaatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcagtgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatagatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgatacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctccccttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagatgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagccagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaggccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatctcatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcctgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatacaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctaggttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctaacaaagatactatagagaagctagaggaattggcagccatcttatcgatggctctgctcctgggcaaaataggatcaatactggtgattaagcttatgcctttcagcggggattttgttcagggatttataagttatgtagggtcccattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatagattaaagaaaactttgaaaatacgaagtttctattcccagctttgtctggt

SEQ ID NO: 60 - DNA, 24061 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggacaaatacttttcacatgatgatccaattagtagtgatcaatccaggttcggatggttcgagaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgaggaccagaacaacatccgcctaccctccatcattgttataaaaaacttaggaaccaggtccacacagccgccagcccatcaaccatccactcccacgattggagccgatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattatgtttatgatcacagcggtgaagcggttaagggaatccaagatgctgactctatcatggttcaatcaggccttgatggtgatagcaccctatcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtatgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagccaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggtcgatcgctccgatacaaccgaccacctacagtgatggcaggctggtgccccaggtcagagtcatagatcctggtctaggcgacaggaaggatgaatgctttatgtacatgtctctgctgggggttgttgaggacagcgatcccctagggcctccaatcgggcgagcatttgggtccctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccactgaccaagcgagaggccagccagcagccgacggcaagcacgaacaccaggcggccccagcacagaacagccctgatacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacctaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgataactgcccggtagtcgaggtgaacggcgtgaccatccaagtcgggagcaggaggtatccagacgctgtgtacttgcacagaattgacctcggtcctcccatattattggagaggttggacgtagggacaaatctggggaatgcaattgctaagttggaggatgccaaggaattgttggagtcatcggaccagatattgaggagtatgaaaggtttatcgagcacttgcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcaccgggaagattctatcacaattccctatcagggatcagggaaaggtgtcagcttccagctcgtcaagctaggtgtctggaaatccccaaccgacatgcaatcctgggtcaccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccaccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaactaatctcatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcctgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatacaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccaggggcctgcccaccaattcgaggtctaagaccggtagagaaatgtgcagttctaaccgaccatatcaaggcagaggctaggttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctaacaaagatactatagagaagctagaggaattggcagccatcttatcgatggctctgctcctgggcaaaataggatcaatactggtgattaagcttatgcctttcagcggggattttgttcagggatttataagttatgtagggtcttattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccgaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattgattgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 61 - DNA, 24061 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcattttctagtgcacttaggattcaagatcctattatcagggacaagagcaggattaaggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgagaacaaggaaatctcagatattgaagtgcaagaccctgagggattcaacatgattctgggtaccatcctagctcaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccctccatcattgttataaaaaacttaggaaccaggtccacacagccgccagcccatcaaccatccactcccacgattggagccgatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattatgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcctccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggttgatcgctccgatacaacccaccacctacagtgatggcaggctggtgccccaggtcagagtcatagatcctggtctaggcgacaggaaggatgaatgctttatgtacatgtttctgctgggggttgttgaggacagcgatcccctagggcctccaatcgggcgagcatttgggtccctgcccttaggtgttggcaaatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcagtgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatagatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgatacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctccccttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagatgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagccagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaggccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatctcatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcctgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatacaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctaggttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctaacaaagatactatagagaagctagaggaattggcagccatcttatcgatggctctgctcctgggcaaaataggatcaatactggtgattaagcttatgcctttcagcggggattttgttcagggatttataagttatgtagggtcccattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatagattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 62 - DNA, 15894 bp, artificial sequence
  synthetic sequence
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 63 - DNA, 31645 bp, artificial sequence
  synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgagagcggtaccccaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtcaagggcaccatcgataacgcggacctggccaaactggcctttaagcggtcatctaagtatgaccttgaatgcgctcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccatgctcccagccaccttgcacgccctgctgctacgagaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtcctaaaggcgaaactctgacggtgggattcactgacagtaggaagattagtcactcatgtacgcacccatttcaccacgaccctcctgtgataggtcgggagaaattccattcccgaccgcagcacggtaaagagctaccttgcagcacctacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccggaaaggaaaaattcacatcccgtttccgctggcaaatgtaacatgcagggtgcctaaagcaaggaaccccaccgtgacgtatggcaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcacgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtcccattcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcttacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccttacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgcccttcggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaataggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgatgagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggtcaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggattatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtctaattcgaactacagctcaacttacctgccaaccccatgccagtcgacccaactagtacaacctaaatccattataaaaaacttaggaaccaggtccacacagccgccagcccatcaaccatccactcccacgattggagcggccgcatggagaagaagcggagaggagcagacacaagcgtgggaatcgtgggcctgctgctgaccacagcaatggcagcagaggtgaccaggagaggaagcgcctactatatgtacctggacaggaatgatgccggcgaggccatctccttcccaaccacactgggcatgaacaagtgctacatccagatcatggacctgggccacatgtgcgatgccaccatgtcctatgagtgtccaatgctggacgagggcgtggagcccgacgatgtggattgctggtgtaataccacatctacatgggtggtgtacggcacctgtcaccacaagaagggagaggcccggcggagccggcgggccgtgacactgccttcccactctaccaggaagctgcagacacgcagccagacctggctggagtccagagagtataccaagcacctgatcagggtggagaactggatctttcgcaatccaggattcgcactggcagcagcagcaatcgcatggctgctgggaagctccaccagccagaaagtgatctacctggtcatgatcctgctgatcgctcctgcctattctatccggtgcatcggcgtgagcaatagagacttcgtggagggaatgtccggaggaacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcaggtgtccaacccagggagaggcatacctggacaagcagtccgatacacagtacgtgtgcaagcggaccctggtggacagaggctggggcaatggctgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactctccaagagccgaggccaccctgggaggatttggctctctgggcctggactgcgagcctagaacaggcctggacttctccgatctgtactatctgaccatgaacaataagcactggctggtgcacaaggagtggtttcacgacatcccactgccatggcacgcaggagcagatacaggaacaccacactggaacaataaggaggccctggtggagttcaaggatgcccacgccaagcggcagacagtggtggtgctgggcagccaggagggagcagtgcacaccgccctggcaggcgccctggaggcagagatggacggagctaagggcagactgtctagcggccacctgaagtgcaggctgaagatggataagctgcgcctgaagggcgtgtcctactctctgtgcacagccgccttcaccttcaccaagatccctgccgagacactgcacggcacagtgaccgtggaggtgcagtatgccggcacagacggaccctgtaaggtgcctgcccagatggccgtggatatgcagacactgacacctgtgggcaggctgatcaccgccaatccagtgatcacagagtctaccgagaacagcaagatgatgctggagctggacccaccatttggcgatagctatatcgtgatcggcgtgggcgagaagaagatcacacaccactggcaccgcagcggctccacaatcggcaaggcctttgaggcaaccgtgcgcggagcaaagagaatggccgtgctgggcgacaccgcatgggatttcggatctgtgggaggcgccctgaacagcctgggcaagggcatccaccagatcttcggcgccgcctttaagtccctgttcggcggcatgagctggttctcacagatcctgatcggcacactgctgatgtggctgggcctgaacaccaagaatggctctatcagcctgatgtgcctggccctgggaggcgtgctgatcttcctgtccaccgccgtgtctgcctgatgagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtact

SEQ ID NO: 64 - DNA, 31676 bp, artificial sequence
  synthetic sequence
ggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggcccgccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagtgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgctcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgacgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgagagcggtaccccaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtcaagggcaccatcgataacgcggacctggccaaactggcctttaagcggtcatctaagtatgaccttgaatgcgctcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccatgctcccagccaccttgcacgccctgctgctacgagaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtcctaaaggcgaaactctgacggtgggattcactgacagtaggaagattagtcactcatgtacgcacccatttcaccacgaccctcctgtgataggtcgggagaaattccattcccgaccgcagcacggtaaagagctaccttgcagcacctacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccggaaaggaaaaattcacatcccgtttccgctggcaaatgtaacatgcagggtgcctaaagcaaggaaccccaccgtgacgtatggcaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcacgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtcccattcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcttacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccttacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgcccttcggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaatagctacagctcaacttacctgccaaccccatgccagacgacccaactagtacaacctaaatccattataaaaaacttaggaaccaggtccacacagccgccagcccatcaaccatccactcccacgattggacctgcaggatgagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggtcaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggattatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtctaattcgaactacagctcaacttacctgccaaccccatgccagacgacccaactagtacaacctaaatccattataaaaaacttaggaaccaggtccacacagccgccagcccatcaaccatccactcccacgattggagcggccgcatggagaagaagcggagaggagcagacacaagcgtgggaatcgtgggcctgctgctgaccacagcaatggcagcagaggtgaccaggagaggaagcgcctactatatgtacctggacaggaatgatgccggcgaggccatctccttcccaaccacactgggcatgaacaagtgctacatccagatcatggacctgggccacatgtgcgatgccaccatgtcctatgagtgtccaatgctggacgagggcgtggagcccgacgatgtggattgctggtgtaataccacatctacatgggtggtgtacggcacctgtcaccacaagaagggagaggcccggcggagccggcgggccgtgacactgccttcccactctaccaggaagctgcagacacgcagccagacctggctggagtccagagagtataccaagcacctgatcagggtggagaactggatctttcgcaatccaggattcgcactggcagcagcagcaatcgcatggctgctgggaagctccaccagccagaaagtgatctacctggtcatgatcctgctgatcgctcctgcctattctatccggtgcatcggcgtgagcaatagagacttcgtggagggaatgtccggaggaacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcaggtgtccaacccagggagaggcatacctggacaagcagtccgatacacagtacgtgtgcaagcggaccctggtggacagaggctggggcaatggctgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactctccaagagccgaggccaccctgggaggatttggctctctgggcctggactgcgagcctagaacaggcctggacttctccgatctgtactatctgaccatgaacaataagcactggctggtgcacaaggagtggtttcacgacatcccactgccatggcacgcaggagcagatacaggaacaccacactggaacaataaggaggccctggtggagttcaaggatgcccacgccaagcggcagacagtggtggtgctgggcagccaggagggagcagtgcacaccgccctggcaggcgccctggaggcagagatggacggagctaagggcagactgtctagcggccacctgaagtgcaggctgaagatggataagctgcgcctgaagggcgtgtcctactctctgtgcacagccgccttcaccttcaccaagatccctgccgagacactgcacggcacagtgaccgtggaggtgcagtatgccggcacagacggaccctgtaaggtgcctgcccagatggccgtggatatgcagacactgacacctgtgggcaggctgatcaccgccaatccagtgatcacagagtctaccgagaacagcaagatgatgctggagctggacccaccatttggcgatagctatatcgtgatcggcgtgggcgagaagaagatcacacaccactggcaccgcagcggctccacaatcggcaaggcctttgaggcaaccgtgcgcggagcaaagagaatggccgtgctgggcgacaccgcatgggatttcggatctgtgggaggcgccctgaacagcctgggcaagggcatccaccagatcttcggcgccgcctttaagtccctgttcggcggcatgagctggttctcacagatcctgatcggcacactgctgatgtggctgggcctgaacaccaagaatggctctatcagcctgatgtgcctggccctgggaggcgtgctgatcttcctgtccaccgccgtgtctgcctgatgaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgc

(19) United States
(12) Patent Application Publication
(10) Pub. No.: US 2023/0235356 A1
(43) Pub. Date: Jul. 27, 2023

(54) AN IMPROVED MEASLES VIRUS VACCINE VECTOR BASED ON MULTIPLE TANDEM ADDITIONAL TRANSCRIPTION UNITS (ATUS)

(71) Applicant: INSTITUT PASTEUR, PARIS (FR)

(72) Inventors: Phanramphoei NAMPRACHAN-FRANTZ, PARIS (FR); Frederic TANGY, PARIS (FR); Chantal COMBREDET, PARIS CEDEX 15 (FR); Segolene GRACIAS, PARIS (FR)

(21) Appl. No.: 18/002,362

(22) PCT Filed: Jul. 8, 2021
(86) PCT No.: PCT/EP2021/069070

§ 371 Date: Dec. 19, 2022

(30) Foreign Application Priority Data
Jul. 8, 2020 (EP) 20305789.8

(51) Int. Cl. C12N 15/86; A61K 39/12

(52) U.S. Cl. CPC C12N 15/86; A61K 39/12; C12N 2770/24134; C12N 2770/20034; C12N 2760/18443; A61K 2039/5156

(57) ABSTRACT

The application generally relates to enhanced recombinant nucleic acid constructs comprising a cDNA molecule encoding a full length antigenomic (+) RNA strand of a non-segmented negative-sense single-stranded RNA virus for expressing at least one heterologous polypeptide, protein, antigen, or antigenic fragment thereof. The application more particularly relates to constructs with multiple ATUs localized within a single intergenic region of a virus. The application also relates to the association between a construct with multiple ATUs and BAG plasmid to facilitate the introduction and expression of large inserts.

### FIELD OF THE INVENTION

The application generally relates to enhanced recombinant genetic constructs comprising a recombinant virus suitable for expressing at least one polypeptide (wherein this term encompasses protein, antigen, or antigenic fragment thereof). The application also relates to the uses of such genetic constructs or viruses, and more particularly their applications for inducing protection against at least one disease, including measles (MeV or MV).

In particular, the present invention is related to:
Genetic constructs, including cDNA molecule encoding a full length antigenomic (+) RNA strand of a non-segmented negative-sense single-stranded RNA virus and a bacterial artificial chromosome (BAC) comprising such a cDNA molecule, with improved ATUs, allowing the insertion and the expression of large size polynucleotides;
Genetic constructs allowing the expression, in particular the simultaneous expression, of at least 2, preferably at least 3, antigens, the antigens being either inserted within a single position in an intergenic region of the genome of the cDNA molecule, or inserted in different localization in intergenic regions in the genome of the cDNA molecule;
A method for rescuing recombinant measles viruses from the BAC plasmid encoding the nucleotide sequence of the full-length antigenomic (+) RNA strand of measles virus;
A method for cultivating the rescued viruses at 32° C. on approved Vero cells in medium without animal serum.

The means of the invention are more particularly dedicated to the provision of a recombinant nucleic acid construct allowing the expression of large heterologous polynucleotide(s) inserted, especially as a result of cloning, within a cDNA molecule encoding a full length antigenomic (+) RNA strand of a non-segmented negative-sense single-stranded RNA virus and the expression of a heterologous polypeptide encoded by such an inserted heterologous polynucleotide, in particular by a heterologous polynucleotide or multiple heterologous polynucleotides having or respectively having together a size of at least 6 kb, and more preferably of at least 6.5 kb, and most preferably over 7 kb.

The means of the invention are more particularly dedicated to a recombinant nucleic acid construct allowing the expression of a plurality of heterologous polypeptides e.g. proteins, antigens, or antigenic fragments thereof, encoded within the recombinant nucleic acid construct, wherein the nucleic acid encoding such heterologous polypeptides e.g., proteins, antigens, or antigenic fragments thereof has a size over 6 kb, more particularly over 6.5 kb, and most preferably over 7 kb.

The means of the invention are more particularly dedicated to a recombinant nucleic acid construct allowing a similar expression (in terms of number of RNA copies issued from the recombinant nucleic acid construct) of a plurality of heterologous polypeptides e.g., proteins, antigens, or antigenic fragments thereof, in particular wherein the nucleic acid encoding such heterologous polypeptides e.g., proteins, antigens, or antigenic fragments thereof has a size over 6 kb, more particularly over 6.5 kb, and most preferably over 7 kb. The invention also relates to the use of these genetic constructs, recombinant nucleic acid constructs, expression vectors comprising these constructs, viruses and virus-like particles issued from the expression of these constructs for inducing an immunogenic response within a host, especially a human host, against at least one disease, including but not limited to an immunogenic response against a pathogenic virus such as measles virus, Chikungunya virus, Zika Virus, West Nile Virus, Dengue virus, SARS, MERS, coronavirus, in particular SARS-Cov-2, Influenza virus, Ebola virus, Nipas virus, HIV and/or a non-viral pathogen such as parasites in particular Plasmodium species causing malaria, in particular those infecting humans P. vivax, P. falciparum, P. malariae, P. ovale, and P. knowlesi.

The invention also relates to the use of a cDNA molecule encoding a full length antigenomic (+) RNA strand of a non-segmented negative-sense single-stranded RNA virus, in particular a Measles virus, as a vector to express immunogens or epitopes of antigens wherein said immunogens or epitopes encompass or derive from heterologous polypeptides originating from pathogens, in particular viruses or parasites, including in particular immunogens or epitopes thereof of measles virus, Chikungunya virus, Zika Virus, West Nile Virus, Dengue virus, SARS, MERS, coronavirus, in particular SARS-Cov-2, Influenza virus, Lassa virus, Ebola virus, Nipah virus, HIV, and non-viral pathogens such as parasites in particular Mycobacterium tuberculosis and Plasmodium species causing malaria, in particular those infecting humans P. vivax, P. falciparum, P. malariae, P. ovale, and P. knowlesi.

### BACKGROUND OF THE INVENTION

Measles virus has been isolated in 1954 (Enders, J. F., and T. C. Peebles. 1954. Propagation in tissue cultures of cytopathogenic agents from patients with measles. Proc. Soc. Exp. Biol. Med. 86:277-286.). Measles virus is a member of the order mononegavirales, i.e. viruses with a non-segmented negative-strand RNA genome. The non-segmented genome of MV has an anti-message polarity which results in a genomic RNA which is neither translated in vivo or in vitro nor infectious when purified. Transcription and replication of non-segmented (−) strand RNA viruses and their assembly into virus particles have been studied and reported especially in Fields virology (3^{rd} edition, vol. 1, 1996, Lippincott-Raven publishers—Fields B N et al.). Transcription and replication of the measles virus do not involve the nucleus of the infected cells but rather take place in the cytoplasm of host cell. The genome of the MV comprises genes encoding six major structural proteins designated N, P, M, F, H and L, and an additional two non-structural proteins from the P gene, C and V. The gene order is the following: from the 3′ end of the genomic RNA; N, P (including C and V), M, F, H and L large polymerase at the 5′ end. The genome furthermore comprises non-coding regions in the intergenic region M/F. This non-coding region contains approximatively 100 nucleotides of untranslated RNA. The cited genes respectively encode the proteins of the nucleocapsid of the virus or nucleoprotein (N), the phosphoprotein (P), the large protein (L) which together assemble around the genome RNA to provide the nucleocapsid, the hemagglutinin (H), the fusion protein (F) and the matrix protein (M).

Attenuated viruses have been derived from MV virus to provide vaccine strains and in particular from the Schwarz strain. The Schwarz measles vaccine is a safe and efficient vaccine currently available for preventing measles. Besides providing vaccine, strains attenuated measles virus such as the Schwarz strain have shown to be stable and suitable for the design of efficient delivery vector for immunization against other infections caused by pathogens like but not limited to West Nile Virus, Chikungunya virus, Dengue Virus, Lassa Virus, Zika virus. Measles vaccine has been administered to billions of children over the last 30 years and have proved both efficiency and safety. It is produced on a large scale in many countries and is distributed at low cost.

Measles vaccine Schwarz/Moraten strain is WHO approved as an efficient and widely used attenuated MV vaccine strain. To generate an infectious clone, its full-length antigenomic viral+strand cDNA was previously cloned into the pTM plasmid under the control of the T7 RNA promoter (Combredet et al, 2003). Several genetic elements such as hammerhead and hepatitis delta virus ribozymes, and T7 terminator, were added for accurate cleavage to ensure the production of full-length viral RNA. The process of viral recovery consists in transfecting pTM-MVschw together with a plasmid expressing the MV-L gene (MV polymerase) into helper HEK293 cells that constitutively express T7 RNA polymerase, MV-N(Nucleoprotein) and MV-P (Phosphoprotein) (Combredet et al, 2003). Transfected cells are then cocultured with an MV permissive cell line such as Vero cells to amplify virus seed. Rescued virus from the cDNA clone was shown to harbor exactly the same sequence as the parental Schwarz virus strain, in particular the sequence of SEQ ID No. 62. To enable insertion of foreign antigens, several additional transcription units (ATUs) were introduced into pTM-MVschw at various sites in MV genome. Cloning sites can accommodate inserts of up to 6 kb in length as multiples of six base pairs to respect the “rule of six” that is essential for measles genome replication (Calain and Roux, 1993).

Recombinant MV vectors generated from attenuated MV vaccine strains are attractive choices for developing vaccine candidates against other diseases. Due to the helicoidal packaging of its genome, MV vector can accommodate foreign genes up to 6 kb in size, although this is very difficult to achieve with the current plasmid-based vectors, and no publication has so far demonstrated such capacity. Heterologous proteins can be stably expressed at high levels for more than 12 passages, and rescued recombinant viruses retain growth capabilities similar to the original MV strain (Frantz et al., 2018). The MV delivery platform has hence been developed from preclinical stage to clinical proof-of-concept with a preclinical track record of fast and effective adaptability to a variety of pathogens. Preclinical immunogenicity and protection from lethal challenges have been shown in mice and non-human primates (NHPs). Target pathogens include HIV (Guerbois et al., 2009; Lorin et al., 2012), West Nile Virus (WNV) (Despres et al., 2005; Brandler et al., 2012), dengue virus (DENV) (Brandler et al., 2007; Brandler et al., 2010), hepatitis B virus (HBV) (del Valle et al., 2007), human papillomavirus (HPV) (Cantarella et al., 2009), Chikungunya virus (CHIKV) (Brandler et al., 2013), Nipah virus (NiV) (Yoneda et al., 2013), respiratory syncytia virus (RSV) (Yamaji et al., 2014), SARS-CoV (Escriou et al., 2014), MERS-CoV (Malczyk et al., 2015), H5N1 influenza A virus (IAV) (unpublished), Zika virus (ZIKV) (unpublished), Lassa virus (LASV) (Mateo et al., 2019) and Ebola virus (EBOV) (unpublished).

Immune protection against several diseases has been reached after vaccination with Measles-derived recombinant vaccines, and protection from lethal infectious challenges has been shown in mice and non-human primates (NHPs). Several recombinant MV vaccines reached clinical trials phase I for treating HIV, ZIKA, and Lassa, and phase II for treating chikungunya infections. The recombinant measles-chikungunya (MV-CHIKV) is the most advanced vaccine candidate. It has completed phase I and II clinical trials in adults (Ramsauer et al, 2015, Reisinger et al, 2018) and is now introduced into phase III trials. These clinical studies showed that the vaccine was well tolerated and elicited a robust and functional antibody response after one (90% sero-converters) or two (100% sero-converters) immunizations. Most importantly, the study demonstrated that the immune response to MV and CHIKV was not dampened by previous MV immunization since all volunteers were pre-immune to measles. Moreover, in this phase 2 trial a group of volunteers received a standard MMR vaccination a month before vaccination with the recombinant MV-CHIKV vaccine and did not show statistically different immunization to CHIKV to the others. Thus, MV recombinant vaccines are well suited to immunize measles pre-immune populations. Also, the antibody response to measles was strongly boosted.

As all RNA viruses, and due to the low fidelity of viral RNA polymerases, MV seeds and stocks are composed of a mixture of various individual viral genome sequences named quasispecies (E. Domingo 1985), among which one genomic sequence is usually greatly preponderant and named the mean genomic sequence. The viral genomic sequences of quasispecies may diverge by single nucleotide variations which may account for great variability in growth capacity or stability. As recombinant MV are generated by reverse genetics systems based on the transfection of helper cells with plasmids encoding the full length antigenomic (+) RNA strand of MV, quasispecies are also generated during this process. Therefore, it is of great importance to be able to isolate, clonally select, and identify appropriate virus clonal seeds that grow efficiently and strongly and stably express the additional antigens. For that purpose, the yield of reverse genetics rescue system is critical, as a sufficient number of viral clones must be generated to screen among them the most performant seed in terms of growing capacity, genomic stability, and expression capacity, which are determinant factors for a live vaccine.

However, the current bacterial plasmids used for MV vector, which are usually around 3 kb in size, accommodate the 16 kb of MV genome plus the different additional genetic material, resulting in plasmids that are frequently larger that 20 kb long. This may be the maximum size that currently used bacterial plasmid for the MV delivery platform can accommodate to be stable in bacteria and mammalian cells without recombining. This issue is responsible for the low yield of reverse genetics rescue systems for MV and other mononegavirales currently observed, and for the impossibility to generate recombinant MV harboring more than 5-6 kb additional nucleotides suitable for use in the preparation of seeds and stocks to elaborate vaccines. With the increasing number of vaccine-preventable emerging diseases, single multivalent vaccines inducing protection from several pathogens are desirable. Multivalent vaccines allow reducing the number of administrations and the costs of manufacturing, storage and transportation. MV recombinant vaccines with single immunogen have proven safe and successful in clinical trials. Therefore, MV vector is considered a front-runner platform to create multivalent vaccines against a combination of diseases, particularly diseases that co-circulate in the same epidemic region such as chikungunya, dengue and Zika fevers for example. For all these reasons, alternatives to the backbone plasmid vector currently used to harbor MV antigenome were considered by the inventors.

Moreover, there is a need for an improved recombinant MV vaccine, allowing the provision of larger antigens and/or more antigens within a single construction, while the construct allows to finely tune the expression gradient of each antigen by providing more insertion sites, allowing to express at the same or at different levels the antigens inserted in the construction. The current MV vaccine vectors do not allow the provision of genetic constructs wherein a plurality of antigenic polypeptides originating from a pathogen or from a plurality of pathogens are individually inserted within a transcription unit and are expressed at the same level. The current state of the art provides such plurality of antigenic polypeptides within a fusion protein or allows the expression of these antigenic polypeptides at different expression levels. However, expressing each antigenic polypeptide in a proper immunogenic configuration may benefit from their individual expression rather than in the form of fusion antigens. Allowing the expression of a plurality of antigenic polypeptides, each in an individual form (e.g. not within a fusion protein or the like), in particular at the same quantitative level may lead to the provision of a more efficient vaccine, either easily produced, and/or eliciting a more suitable immune response when administered to a host, in particular a human subject. It will also facilitate the manufacturing of single multivalent vaccines rather than manufacturing several individual vaccines to be administered simultaneously.

### ASPECTS AND EMBODIMENTS OF THE INVENTION

### Definitions

By “heterologous”, it should be understood that the heterologous polypeptide is encoded by a nucleotide sequence provided, especially recombined into the cDNA molecule encoding a full length antigenomic (+) RNA strand of a non-segmented negative-sense single-stranded RNA virus, while it is not naturally present in such cDNA molecule. Heterologous polynucleotides derive from an organism different from the organism from which the cDNA molecule encoding a full length antigenomic (+) RNA strand of a non-segmented negative-sense single-stranded RNA virus is issued. In a particular embodiment, the heterologous polynucleotide encoding the heterologous polypeptide is cloned from or derived from a different virus species from that of the recipient full-length antigenomic (+) RNA; in particular, the heterologous polynucleotide encodes a heterologous polypeptide that is not naturally encoded by a measles virus. Typically in order to elicit in a host, an immune response against the pathogen or the infection caused by the pathogen providing the heterologous polypeptide, the polypeptide itself is not transferred to the expressing cells or administered to the host, but instead the genetic material encoding for the polypeptide (the complementary DNA or cDNA) is inserted within the cDNA molecule encoding a full length antigenomic (+) RNA strand of a non-segmented negative-sense single-stranded RNA virus.

An “expression cassette” is a component of the cDNA comprising a coding sequence, in particular an Open Reading Frame or a gene together with operably linked regulatory sequence(s) for expression, to be expressed by a transfected cell, when the nucleic acid construct comprising the cDNA is present within the transfected cell. The expression cassette allows the transfected cell's machinery to make RNA and protein(s) from the nucleic acid construct of the invention. In addition to the genes of the cDNA molecule and the inserted polynucleotides encoding heterologous polypeptides, an expression cassette usually comprises at least one of the following elements: (inducible) promoters, multiple cloning site(s), an efficient terminator of transcription. An expression cassette may therefore be defined as a polynucleotide present within the cDNA molecule and composed of one or more ORFs or genes and the sequences controlling their expression within the cDNA. An expression cassette comprises at least three components: a promoter sequence, an Open Reading Frame, and a 3′ untranslated region that, in eukaryotes, usually contains a polyadenylation site.

A “bacterial artificial chromosome plasmid” (also referenced as “BAC” or BAC plasmid or recombinant BAC plasmid in the present description) is a DNA construct, based on a functional fertility plasmid (or F-plasmid). Recombinant BAC plasmid usually comprises a sequence for plasmid replication and regulation of copy number (repE gene); genes for partitioning the F plasmid into daughter cells during division and to ensure stability of the recombinant BAC plasmid (parA and parB genes); a selectable marker giving antibiotic resistance to cells comprising the BAC, and promoters for transcription of gene(s) possibly inserted therein. BAC plasmids have a high capacity for cloning of large sequences of DNA that can be further modified. The product is a supercoiled, circular molecule that is resistant to rearrangement and shearing. Low-copy BAC replicons were particularly used to clone large DNA or RNA viruses of up to 300 kb (Embers et al.) for example, Human herpes virus HHV-6A (Borenstein et al. 2009), poxviruses (Cottingham et al. 2008; Domi et al. 2002; Meisinger-Henschel et al. 2010; Roth et al. 2011), coronaviruses (Almazan et al. 2000, Almazan et al. 2006; Fan et al. 2008; Rasmussen et al. 2010; Jengarn et al. 2015), or flaviviruses (Pu et al. 2011; Aubry et al. 1989). Moreover, BAC plasmids allow an easier cloning of toxic viral proteins in bacterial cells (O'Connor et al.).

The recombinant BAC plasmid may be selected from the list consisting of the pSMART BAC plasmid or any BAC plasmid issued from the pSMART BAC plasmid, pEZ-BAC plasmid, pBeloBACllplasmid (from New England Biolabs), pBACe3.6 (from New England Biolabs), pBAC/OriV, which is issued from pBeloBAC11 and is disclosed in Wild et al. Genome Res. 2002. 12: 1434-1444), pBAC-RT (disclosed in Stavropoulos et al, Genomics 2001 Feb. 15; 72(1):99-104. doi: 10.1006/geno.2000.6481), pHA1 and pHA2 (both disclosed in Adler et al, J Virol. 2000 August; 74(15):6964-74. doi: 10.1128/jvi.74.15.6964-6974.2000), pTARBAC2 disclosed in Zeng et al, Genomics. 2001 September; 77(1/2):27-34 and its derivatives disclosed in Fuentes et al G3 (Bethesda). 2016 Jan. 27. doi: 10.1534/g3.115.026344), the pBAC contruct inserted in Transmissible gastroenteritis coronavirus (TGEV) (disclosed in Sola et al., J Virol. 2003 April; 77(7):4357-69). In a preferred embodiment of the invention, the recombinant BAC plasmid is selected from the list consisting of the pSMART-BAC, the pBeloBAC11 and the pBACe3.6. In a more preferred embodiment, the recombinant BAC plasmid is the pSMART-BAC.

The expression “encodes” used in the present description encompasses the ability of a cDNA molecule to allow transcription of a full length antigenomic (+) RNA, said cDNA serving especially as a template for transcription and where appropriate translation for product expression into cells or cell lines. Hence, when the cDNA is a double stranded molecule, one of the strands has the same nucleotide sequence as the antigenomic (+) RNA of the RNA virus, especially of the measles virus, with the first heterologous polynucleotide cloned within, except “U” nucleotides that are substituted by “T” nucleotides in the cDNA. The nucleic acid construct of the invention may comprise regulatory elements controlling the transcription of the coding sequences, in particular promoters and termination sequences for the transcription, and possibly enhancer and other cis-acting elements. These regulatory elements may be heterologous with respect to the heterologous polynucleotide issued or derived from the polypeptide encoded by the inserted polynucleotide within the cDNA of the MV, and in particular may be the regulatory elements of the measles virus strain, e.g., promoters, enhancers, cis-acting elements of the Measles virus.

The expressions “operatively inserted”, “operably inserted” which may be substituted by the expression “operatively/operably linked” or “operatively/operably cloned”, refer to the functional cloning, or insertion, of a heterologous polynucleotide within the nucleic acid construct of the invention in particular within the cDNA encoding a full length antigenomic (+) RNA strand of a non-segmented negative-sense single-stranded RNA virus, especially of a measles virus, such that said heterologous polynucleotide and nucleic acid construct are effectively, or efficiently, transcribed and if appropriate translated, in particular in cells, cell line, host cell used as a part of a rescue system for the production of recombinant infectious virus particles, especially recombinant infectious MV particles or MV expressing at least one heterologous polypeptide, e.g., at least one protein, or at least one antigen, or at least an antigenic fragment thereof, of the heterologous polynucleotide inserted within the cDNA molecule encoding a full length antigenomic (+) RNA strand of a non-segmented negative-sense single-stranded RNA virus, especially of the Measles virus. In other words, the nucleic acid construct of the invention allows the production, when placed in appropriate conditions, of an infectious antigenomic (+) RNA capable of producing at least one heterologous polypeptide, e.g. at least one protein, or at least one antigen, or at least an antigenic fragment thereof encoded by the heterologous polynucleotide inserted within the cDNA molecule encoding a full length antigenomic (+) RNA strand of a non-segmented negative-sense single-stranded RNA virus, especially of the Measles virus.

A “pathogen” may be any kind of pathogen according to its broadest definition; a pathogen may be any microorganism that can alter health of a host, in particular produce disease. In particular, pathogens are selected among the pathogens that infect human. A pathogen may also be referred to as an infectious agent, or simply as a germ. A pathogen may be a virus, in particular a virus infecting human, especially a virus selected from the list consisting of measles virus, Chikungunya virus, Zika Virus, West Nile Virus, Dengue virus, SARS, MERS, coronavirus, in particular SARS-Cov-2, Influenza virus, Ebola virus, Nipas virus, HIV. A pathogen may alternatively be a non-viral pathogen such as a parasite in particular Mycobacterium tuberculosis and Plasmodium species causing malaria, in particular those infecting humans including P. vivax, P. falciparum, P. malariae, P. ovale, and P. knowlesi.

The term “polypeptide” is used to designate for example an “antigen” or a “protein” or “antigenic fragment” and defines a molecule resulting from a concatenation of amino acid residues. In particular, the polypeptides disclosed in the application originate from a pathogen and are antigens, proteins, structural proteins, or antigenic fragments thereof, that may be identical to native proteins or alternatively that may be derived thereof by mutation, including by substitution (in particular by conservative amino acid residues) or by addition of amino acid residues or by secondary modification after translation or by deletion of portions of the native proteins(s) resulting in fragments having a shortened size with respect to the native protein of reference. Fragments are encompassed within the present invention to the extent that they bear epitopes of the native protein suitable for the elicitation of an immune response in a host in particular in a human host, including a child host, preferably a response that enables the protection against an infection from a pathogen or against a pathogen-associated disease.

Epitopes are in particular of the type of T epitopes involved in elicitation of Cell Mediated Immune response (CMI response). T epitopes are involved in the stimulation of T cells through presentation of some parts of the T-cell epitope which can bind on MHC class II molecules, leading to the activation of T cells. Epitopes may alternatively be of type B, involved in the activation of the production of antibodies in a host to whom the protein has been administered or in whom it is expressed following administration of the infectious replicative particles of the invention. Fragments may have a size representing more than 50% of the amino-acid sequence size of the native protein of the pathogen wild type strain, preferably at least 90% or at least 95%. Polypeptide may have at least 50% identity with the native protein of the pathogen wild type strain, preferably at least 60%, preferably at least 70%, preferably at least 85%, at least 95%, at least 98% or at least 99% when comparison is carried out after alignment of the fragment or the modified amino acid sequences of the polypeptide with the corresponding region of the native protein.

Nucleic Acid Constructs with Multiple Additional Transcription Units (ATUs)

In a first aspect the invention provides a nucleic acid construct which comprises a cDNA molecule encoding a full length antigenomic (+) RNA strand of a non-segmented negative-sense single-stranded RNA virus, especially of a measles virus (MV), wherein the cDNA molecule further comprises inserted therein, at least a first additional transcription unit (ATU) and a second additional transcription unit (ATU), wherein the at least first and second additional transcription units (ATUs) are localized within a single intergenic region of the cDNA encoding the antigenomic (+) RNA strand as a single expression cassette, and wherein each ATU comprises a heterologous polynucleotide operably inserted within the ATU allowing the expression of a heterologous polypeptide encoded by the heterologous polynucleotide. Nucleic acid constructs according to this aspect of the invention are illustrated in FIGS. 1 to 8 and 9 to 13 for the measles virus.

According to a particular embodiment of the invention, Additional Transcription Units (ATU) comprises cis-acting sequences encompassing a promoter sequence and a terminator sequence originating from intergenic regions of measles virus genome inserted respectively upstream and downstream of additional heterologous sequences and allowing their expression by the measles RNA-dependent RNA polymerase. These cis-acting sequences can be selected from any intergenic sequence of measles virus genome: N-P, P-M, M-F, F-H, H-L. For the purpose of the description of the invention and depending on the context of the disclosure, the term “ATU” is used to designate the herein defined cis-acting sequences and is also used to designate the nucleic acid sequence framed by said cis-acting sequences and obtained after inserting the polynucleotide(s) that encodes the heterologous polypeptide(s).

The nucleic acid construct of the invention is in particular a purified DNA molecule, obtained or obtainable by recombination of at least one polynucleotide of a non-segmented negative-sense single-stranded RNA virus, especially of MV and at least two heterologous polynucleotides of a pathogen, especially a pathogen infecting human, operably cloned or linked together. Accordingly, in an embodiment of the invention, the nucleic acid construct is a recombinant nucleic acid construct.

According to the invention, the nucleic acid construct is prepared by cloning two polynucleotides, or several polynucleotides, e.g. more than two polynucleotides, each encoding at least one polypeptide e.g. a protein, an antigen, an antigenic fragment thereof, issued from a pathogen, in the cDNA encoding the full-length antigenomic (+) RNA of the virus, especially of the measles virus, said cloning enabling each ATU to contain at least one coding heterologous polynucleotide. As an example, the genome of a measles virus with ATUs is illustrated on FIG. 2 while several nucleic acid constructs according to the invention are illustrated on FIGS. 1, 3-8 and on FIGS. 10-17, and FIG. 25 for the measles virus. Alternatively, a nucleic acid construct of the invention may be prepared using steps of synthesis of nucleic acid fragments or polymerization from a template, including by PCR. The polynucleotide(s) and nucleic acid construct of the invention may be prepared in accordance with any known method in the art and in particular may be cloned, obtained by polymerization especially using PCR methods or may be synthesized.

As illustrated on FIG. 2 for the measles virus, different constructions are encompassed within the definition of the invention, wherein within a single intergenic region of the cDNA for the full length antigenomic (+) RNA strand of a non-segmented negative-sense single-stranded RNA genome of a measles virus, two ATUs may be provided between the P gene and the M gene, or three ATUs may be localized between the P gene and the M gene of the measles virus in the cDNA molecule(as illustrated on the FIG. 2A); two ATUs may be in the intergenic region localized between the H gene and the L gene of the measles virus in the cDNA molecule; three ATUs may be in the intergenic region localized between the H gene and the L gene of the measles virus in the cDNA molecule (as illustrated on the FIG. 2B); two ATUs may be in the intergenic region localized upward the N gene of the measles virus in the cDNA molecule; three ATUs may be localized in the intergenic region upward the N gene of the measles virus in the cDNA molecule (as illustrated on the FIG. 2C). In another embodiment of a single construct, ATUs may also be localized in different intergenic regions of the measles virus in the cDNA molecule; for example, two ATUs may be localized in the intergenic region localized between the P gene and the M gene of the measles virus in the cDNA molecule and one ATU may be localized in the intergenic region localized between the H gene and the L gene of the measles virus in the cDNA molecule (illustrated on FIG. 2D); two ATUs may be localized in the intergenic region localized between the P gene and the M gene of the measles virus in the cDNA molecule and two ATUs may be localized in the intergenic region localized between the H gene and the L gene of the measles virus in the cDNA molecule (illustrated on FIG. 2E); two ATUs may be localized in the intergenic region localized between the P gene and the M gene of the measles virus in the cDNA molecule and two ATUs may be localized in the intergenic region localized upward the N gene of the measles virus in the cDNA molecule (illustrated on FIG. 2F); two ATUs may be localized in the intergenic region localized between the P gene and the M gene of the measles virus in the cDNA molecule and one ATU may be localized in the intergenic region localized upward the N gene of the measles virus in the cDNA molecule, and one ATU may be localized in the intergenic region between the H gene and the L gene of the measles virus in the cDNA molecule (illustrated on FIG. 2G). These embodiments are only illustrative of the invention. Several other embodiments wherein a different number of ATU regions, and/or different localization of ATUs, are encompassed within the present definition of the invention, provided that at least two ATUs are localized within the same intergenic region of the full length antigenomic (+) RNA strand of a non-segmented negative-sense single-stranded RNA virus and each contains (or each is suitable for containing) a heterologous polynucleotide encoding a heterologous polypeptide issued from a pathogen.

As shown on FIG. 2, a plurality of ATUs may be inserted in a single intergenic region of the nucleic acid construct, allowing the insertion within each single ATU of at least one heterologous polynucleotide encoding at least one heterologous polypeptide, thereby allowing the insertion of either one or multiple polynucleotides issued from a single pathogen, or a plurality of heterologous polynucleotides issued from different pathogens. On FIG. 11, antigens originating from the west Nile virus (WNV), the Chikungunya virus (CHIKV) and the Zika Virus (ZIKV) are each encoded by a polynucleotide inserted within its proper ATU localized within the same single intergenic region of the MV genome, i.e., between the P gene and the M gene of the MV genome in the illustrated example. In another embodiment, antigens originating from the West Nile virus (WNV), the Chikungunya virus (CHIKV) and the Zika Virus (ZIKV) are each encoded by a polynucleotide inserted within its proper ATU localized within the same single intergenic region of the MV genome, i.e. between the H gene and the L gene of the MV genome, in the so-called ATU3 illustrated in the figures. Such a construct corresponds to the plasmid illustrated in FIG. 16. In another embodiment, antigens originating from the West Nile virus (WNV), the Chikungunya virus (CHIKV) and the Zika Virus (ZIKV) are each encoded by a polynucleotide, the polynucleotides being inserted in two different ATUs localized in different intergenic regions of the MV genome, i.e. between the P gene and the M gene of the MV genome for the polynucleotide encoding the CHIKV antigen, in the so-called ATU2 illustrated in the figures, and between the H gene and the L gene of the MV genome for the polynucleotides encoding the WNV antigen and the ZIKV antigen. Such a construct corresponds to the plasmid illustrated in FIG. 17.

In a particular embodiment of the invention, illustrated on FIG. 2, a first ATU and a second ATU are localized: (i) between the P gene and the M gene of the measles virus in the cDNA molecule or (ii) between the H gene and the L gene of the measles virus in the cDNA molecule or (iii) between the N gene of the measles virus and the T7RNA polymerase promoter in the cDNA molecule.

An Additional Transcription Unit according to the invention is hence a DNA unit suitable to be cloned (or inserted) or cloned (or inserted) within a cDNA molecule encoding a full length antigenomic (+) RNA strand of a non-segmented negative-sense single-stranded RNA virus, especially of a measles virus, preferably in an intergenic region of the virus genome. The ATU encodes a heterologous polypeptide as defined herein and contains both a coding sequence, which will be translated into the polypeptide, and regulatory sequence(s), which direct and regulate the synthesis of that polypeptide. ATU may furthermore comprise one, two or several restriction sites to ease the insertion of different polynucleotides in a suitable manner for expressing a polypeptide encoded by each inserted polynucleotide. In a particular embodiment of the invention, the ATUs contain cis-acting sequences necessary for the transcription of the P gene of the measles virus.

An ATU generally comprises two CTT codons corresponding respectively to the start and stop codons of the virus polymerase. ATUs may further comprise ATG START codon and STOP codon(s), the STOP codon(s) being selected from TAA, TAG or TGA codons, and is in particular TAG codon, corresponding respectively to the start and stop codons for translation of the heterologous polynucleotide comprised within the ATU. In a particular embodiment, ATG and TAG codons corresponding respectively to the start and stop codons for translation of the heterologous polynucleotide cloned within the ATU are part of the ATU. An ATU may also comprise at least one of the following elements: a TATA box, more particularly of sequence “TTATAAAAAA”, at least one Start/Stop codon, in particular a “CTT” codon, more particularly two “CTT” codons; at least one “CTT” codon may directly follow the TATA box in the 3′ or 5′ direction, a promoter (for example a promoter issued from a RNA virus, especially of a measles virus (MV), more particularly the promoter of the P gene of the MV, or the promoter of the F gene of the MV, or the promoter of the H gene of the MV, or the promoter of the L gene of the MV, or the promoter of the M gene of the MV, or the promoter of the N gene of the MV,), more particularly directly following the Start/Stop codon in the 3′ direction, of the cDNA molecule encoding a full length antigenomic (+) RNA strand of a non-segmented negative-sense single-stranded RNA virus, especially of the MV wherein an ORF is inserted that encodes a heterologous polypeptide. In a preferred embodiment of the invention, an ATU comprises a CTT codon, a promoter sequence, at least one restriction site, a heterologous polynucleotide encoding a heterologous polypeptide (and comprising a START codon and at least one STOP codon), at least one restriction site, a terminator 5 sequence (which could be a TATA box as defined here above), and a CTT codon; in particular in this order from the 5′ end towards the 3′ end of the ATU. Several illustrative embodiments are represented in FIGS. 7, 10 and 18.

In an embodiment of the invention, an ATU comprises from its 5′ end to its 3′ end:
a promoter sequence originating from intergenic regions of measles virus genome,
a heterologous polynucleotide encoding at least one heterologous polypeptide as defined herein, wherein the heterologous polynucleotide is in particular inserted in a multiple cloning site, especially a multiple cloning site containing restriction sites not found in the MV cDNA or rarely found therein;
a terminator sequence,
wherein the promoter and terminator sequences are inserted respectively upstream and downstream of additional heterologous sequences in a way to allow their expression by the measles RNA-dependent RNA polymerase and wherein the promoter and terminator sequences can be selected from the functional sequences in any intergenic sequence of measles virus genome: N-P, P-M, M-F, F-H, H-L

In a particular embodiment of the invention, the 5′ extremity of an ATU (i.e. the extremity upward the heterologous polynucleotide encoding the heterologous polypeptide) comprises a promoter sequence selected from:
SEQ ID No. 40 (promoter of the gene N of MV); or
SEQ ID No. 41 (promoter of the gene P of MV); or
SEQ ID No. 42 (promoter of the gene M of MV); or
SEQ ID No. 43 (promoter of the gene F of MV); or
SEQ ID No. 44 (promoter of the gene H of MV); or
SEQ ID No. 45 (promoter of the gene L of MV).

In a particular embodiment of the invention, the 3′ extremity of an ATU (i.e. the extremity downward the heterologous polynucleotide encoding the heterologous polypeptide) comprises a terminator sequence selected from:
SEQ ID No. 46 (terminator of the N gene of MV); or
SEQ ID No. 47 (terminator of the P gene of MV); or
SEQ ID No. 48 (terminator of the M gene of MV); or
SEQ ID No. 49 (terminator of the F gene of MV); or
SEQ ID No. 50 (terminator of the H gene of MV); or
SEQ ID No. 51 (terminator of the L gene of MV).

In a particular embodiment of the invention, the 5′ extremity of an ATU (i.e. the extremity upward the heterologous polynucleotide encoding the heterologous polypeptide) and the 3′ extremity of an ATU (i.e. the extremity downward the heterologous polynucleotide encoding the heterologous polypeptide) respectively comprise:
SEQ ID No. 40 (promoter of the N gene of MV) and SEQ ID No. 46 (terminator of the N gene of MV); or
SEQ ID No. 41 (promoter of the P gene of MV) and SEQ ID No. 47 (terminator of the P gene of MV); or
SEQ ID No. 42 (promoter of the M gene of MV) and SEQ ID No. 48 (terminator of the M gene of MV); or
SEQ ID No. 43 (promoter of the F gene of MV) and SEQ ID No. 49 (terminator of the F gene of MV); or
SEQ ID No. 44 (promoter of the H gene of MV) and SEQ ID No. 50 (terminator of the H gene of MV); or
SEQ ID No. 45 (promoter of the L gene of MV) and SEQ ID No. 51 (terminator of the L gene of MV).

In a particular embodiment of the invention, an ATU comprises or consists in the sequence of:
SEQ ID No. 52, wherein the n corresponds to the heterologous polynucleotide encoding a heterologous polypeptide; or
SEQ ID No. 53, wherein the n corresponds to the heterologous polynucleotide encoding a heterologous polypeptide; or
SEQ ID No. 54, wherein the n corresponds to the heterologous polynucleotide encoding a heterologous polypeptide; or
SEQ ID No. 55, wherein the n corresponds to the heterologous polynucleotide encoding a heterologous polypeptide; or
SEQ ID No. 56, wherein the n corresponds to the heterologous polynucleotide encoding a heterologous polypeptide; or
SEQ ID No. 57, wherein the n corresponds to the heterologous polynucleotide encoding a heterologous polypeptide.

The heterologous sequence represented by “n” does not need a START codon, since the START codon is already present upward the “n’ in the sequence SEQ ID No. 52, SEQ ID No. 53, SEQ ID No. 54, SEQ ID No. 55, SEQ ID No. 56, SEQ ID No. 57. A STOP codon may or may not be present in the heterologous polynucleotide represented by n since a STOP codon is already present in SEQ ID No. 52 downward the “n” in the sequence; an extra STOP codon may nonetheless be added, in particular for compliance with the rule of 6. The “n” feature is not intended to provide indication relative to the number of nucleotides contained in the sequence of the heterologous polynucleotide but rather to indicate the location of the heterologous polynucleotide in the ATU sequence.

In a particular embodiment of the invention, an ATU comprises from its 5′ end to its 3′ end:
A polynucleotide (5′ATU) comprising or consisting of (i) SEQ ID No. 16 (5′tataaaaaacttaggaaccaggtccacacagccgccagcccatcaacgcgtacgATG 3′); or (ii) SEQ ID No. 19 (5′tataaaaaacttaggaaccaggtccacacagccgccagcc
catcaaccatccactcccacgattggaggccggccATG 3′), or (iii) SEQ ID No. 20 (5′ tataaaaaacttaggaaccaggtccacacagccgccagcccatcaaccatccactcccacgattg gagcggccgcATG 3′);
A heterologous polynucleotide encoding at least one heterologous polypeptide as defined herein;
A polynucleotide (3′ATU) comprising or consisting of SEQ ID No. 17 (5′ (TAA)TAGgcgcgcagcgcttagacgtctcgcgaTCGATACTAGTACAACCTAAAT CCATTATAAAAAACTTAGGAGCAAAGTGAT 3′) (characters in bold and in lowercase are extra nucleotide residues added to respect the rule of 6, underlined residues correspond to restriction sites (for BssHII, AfeI and NruI).

The combination of these three polynucleotides forms an ATU, allowing the transcription and the expression of the heterologous polypeptide encoded by the heterologous polynucleotide. The polynucleotide of SEQ ID No. 16 comprises the TATA box, the CTT codon, the P promotor, a restriction site for BsiWI restriction enzyme, and a START codon, while the polynucleotide of SEQ ID No. 17 comprises a STOP codon, and a CTT codon. Additional nucleotides may be present within an ATU, preferentially not between the polynucleotide of SEQ ID No. 16 (or SEQ ID No. 19 or SEQ ID No. 20) and the heterologous polynucleotide and not between the heterologous polynucleotide and the polynucleotide of SEQ ID No. 17. An extra STOP codon TAA may be present towards the 5′ end of SEQ ID No. 17, in particular for compliance with the rule of 6. Additional nucleotides may be present at the 5′ end and/or at the 3′ end of the ATU. Such additional nucleotides may for example be restriction sites or regulation sequences, like but not limited to silencers, enhancers, promoters, terminators.

In a particular embodiment, the nucleic acid construct of the invention comprises a cDNA molecule encoding a full length antigenomic (+) RNA strand of a non-segmented negative-sense single-stranded RNA virus, especially of a measles virus (MV), wherein the cDNA molecule comprises a first ATU and a second ATU localized within a same intergenic region of the cDNA of the virus, wherein the first ATU and the second ATU each comprise or consist from their 5′ end towards their 3′ end of (i) the nucleotide sequence of SEQ ID No. 16 or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45, (ii) a heterologous polynucleotide sequence encoding a heterologous polypeptide, and (iii) the nucleotide sequence of SEQ ID No. 17 or SEQ ID No. 46 or SEQ ID No. 47 or SEQ ID No. 48 or SEQ ID No. 49 or SEQ ID No. 50 or SEQ ID No. 51, wherein the heterologous polynucleotides present within the first ATU and the second ATU are different. The intergenic region comprising the ATUs may in particular be any of those cited herein in respect of the full length antigenomic (+) RNA strand of MV.

In a particular embodiment, the nucleic acid construct of the invention comprises a cDNA molecule encoding a full length antigenomic (+) RNA strand of a non-segmented negative-sense single-stranded RNA virus, especially of a measles virus (MV), wherein the cDNA molecule comprises a first ATU and a second ATU localized within a same intergenic region of the cDNA of the virus, wherein the first ATU and the second ATU each comprise or consist from their 5′ end towards their 3′ end of (i) the nucleotide sequence of SEQ ID No. 52, SEQ ID No. 53, SEQ ID No. 54, SEQ ID No. 55, SEQ ID No. 56, SEQ ID No. 57, wherein n corresponds to a heterologous polynucleotide sequence encoding a heterologous polypeptide, and, wherein the heterologous polynucleotides present within the first ATU and the second ATU are different. The intergenic region comprising the ATUs may in particular be any of those cited herein in respect of the full length antigenomic (+) RNA strand of MV.

In a particular embodiment of the invention, an ATU is a polynucleotide comprised within SEQ ID No: 1. SEQ ID No. 1 comprises an ATU sequence suitable for cloning within the cDNA molecule encoding a full-length antigenomic (+) RNA strand of a non-segmented negative-sense single-stranded RNA MV virus, wherein a heterologous polynucleotide sequence encoding a heterologous polypeptide issued or derived from a pathogen is inserted between positions 79 and 796 included (i.e. comprising the initiation codon ATG present at the 3′ end of the sequence SEQ ID No. 16, SEQ ID No. 19 and SEQ ID No. 20, a stop codon TAA is present at positions 794 to 796 of SEQ ID No. 1 and corresponds to the native stop codon of the polynucleotide sequence; another stop codon TAG is also present at positions 797 to 799 of SEQ ID No: 1 which corresponds to the STOP codon present in SEQ ID No. 17) or between positions 82 and 796 included (i.e. without the start codon ATG present at the 3′ end of the sequence SEQ ID No. 16, SEQ ID No. 19 and SEQ ID No. 20). TATA box and CTT codons encompassing respectively the start and stop codons of the polymerase are in bold. ATG and TAG codons corresponding to the start and stop codons for translation of the heterologous polynucleotide cloned within the ATU are underlined.

(SEQ ID No. 16 * SEQ ID No. 17)
ctagcctaccctccatcattgttataaaaaacttaggaaccaggtccaca
cagccgccagcccatcaacgcgtactATG*TAATAGcgcgcagcgcttag
acgtctcgcgaTCGATACTAGTACAACCTAAATCCATTATAAAAAACTTA
GGAGCAAAGTGAT.

The ATG codon in bold corresponds to the start codon ATG of the heterologous polypeptide and is provided at the 3′ end of sequence SEQ ID No. 16 in the present example. This start codon ATG corresponds to the first codon of the coding sequence of the heterologous polypeptide. Underlined codons are STOP codons, characters in lowercase correspond to the restriction sites, CTT codons are in bold and underlined.

The * sign in the sequence corresponds to the location of the insert of a heterologous polynucleotide. In the polynucleotide of SEQ ID No. 1, * is a sequence encoding Green Fluorescent Protein (GFP) (minus the START codon ATG localized at positions 79 to 81 of SEQ ID No. 1 and already represented here above in bold capital characters) and the STOP codons of the sequence localized at positions 794 to 796 of SEQ ID No. 1 and already represented here above in underlined capital characters) localized between the nucleotide at position 79 and the nucleotide at position 796. In the present invention, this sequence localized between the nucleotide at position 79 and the nucleotide localized at position 796 may be substituted by any heterologous polynucleotide encoding a heterologous polypeptide issued from a pathogen. In other words, the * sign may be substituted by any heterologous polynucleotide encoding a heterologous polypeptide, and an ATU may comprise or consist of the heterologous polynucleotide of SEQ ID No. 1 wherein the nucleotide sequence from position 79 to position 796 is substituted by a heterologous polynucleotide sequence encoding a heterologous polypeptide, in particular wherein said heterologous polynucleotides in the first ATU and the second ATU encode heterologous polypeptides that are different from each other and optionally wherein the first and the second heterologous polynucleotides ATUs are separated in the single intergenic region of the cDNA by a spacer sequence such as the sequence of polynucleotide of SEQ ID No. 2 or SEQ ID No. 11; the spacer sequence consisting of the 3′ end of the first ATU followed by the 5′ end of the subsequent ATU (i.e. the second ATU). As an example, an ATU corresponding to this definition may be retrieved from the polynucleotide of SEQ ID No. 12 which 5 corresponds to an expression vector wherein a heterologous polynucleotide encoding an immunogenic polypeptide of CHIKV is localized between nucleotide residues 3532 and 7275 on SEQ ID No. 12. This heterologous polynucleotide, encoding an immunogenic polypeptide of Chikungunya virus takes place of the * sign in the ATU defined here above and is framed towards its 5′ end and its 3′ end by the polynucleotide sequence illustrated here above.

Alternatively, an ATU according to the invention may correspond to a polynucleotide sequence wherein ATU localized within SEQ ID No. 1 may be substituted by one of the following ATU:

(SEQ ID No. 19 * SEQ ID No. 17)
tataaaaaacttaggaaccaggtccacacagccgccagcccatcaacca
tccactcccacgattggaggccggccATG*TAATAGgcgcgcagcgctt
agacgtctcgcgaTCGATACTAGTACAACCTAAATCCATTATAAAAAAC
TTAGGAGCAAAGTGAT.
(SEQ ID No. 20 * SEQ ID No. 17)
tataaaaaacttaggaaccaggtccacacagccgccagcccatcaacca
tccactcccacgattggagcggccgcATG*TAATAGgcgcgcagcgctt
agacgtctcgcgaTCGATACTAGTACAACCTAAATCCATTATAAAAAAC
TTAGGAGCAAAGTGAT.

Underlined codons are STOP codons, characters in lowercase correspond to the restriction sites, CTT codons are in bold and underlined.

The nucleotide residues next to the 5′ end and the 3′ end of each ATU may correspond to the same residues as the nucleotide residues next to the 5′ end and the 3′ end of the ATU localized within SEQ ID No. 1, or may differ, as illustrated in SEQ ID No. 13, wherein the nucleotide residues next to the 5′ end and 3′ end of each ATU present within SEQ ID No. 13 are not identical.

In another particular embodiment, within a single intergenic region of the full length antigenomic (+) RNA strand of a non-segmented negative-sense single-stranded RNA virus, especially of MV, comprising several heterologous polynucleotides inserted therein, the first heterologous polynucleotide encoding a first heterologous polypeptide and the second (and/or subsequent) heterologous polynucleotide encoding second (and/or subsequent) heterologous polypeptide may be separated by a polynucleotide of sequence SEQ ID No. 2 or SEQ ID No. 11.

The polynucleotides of SEQ ID No. 2 and SEQ ID No. 11 comprise from their 5′ end towards their 3′ end a STOP codon, a first CTT codon, a TATA box, a second CTT codon and a P promoter. They comprise elements found at the end of a first ATU and at the beginning of a subsequent ATU. The polynucleotide of SEQ ID No. 2 or of SEQ ID No. 11 may separate two heterologous polynucleotides each encoding a heterologous polypeptide and each pertaining to separate ATUs, as a result of insertion of the sequence of SEQ ID No. 2 or 11. In other words, in the nucleic acid construct, and when two heterologous polynucleotides are localized within the same intergenic region of the cDNA of the virus, and when a polynucleotide of SEQ ID No. 2 or of SEQ ID No. 11 is inserted between said two heterologous polynucleotides, the 5′ end (at least the STOP codon and the first CTT codon) of the polynucleotide of SEQ ID No. 2 or of SEQ ID No. 11 belongs to the first ATU, comprising the first heterologous polynucleotide while the 3′ end (at least the TATA box, the second CTT codon and the P promoter) of such sequence belongs to the second ATU comprising the second heterologous polynucleotide.

In an embodiment of the invention, the first ATU and the second ATU, both localized within a same intergenic region of the cDNA of the virus, comprise or consist in the nucleotide sequence of SEQ ID No. 16 or SEQ ID No. 19 or SEQ ID No. 20, a heterologous polynucleotide encoding a heterologous polypeptide as defined here in (said sequence comprising a start codon ATG, since SEQ ID No. 16, SEQ ID No. 19 and SEQ ID No. 20 all comprise the start codon ATG at their 3′ end), and the nucleotide sequence of SEQ ID No. 17; optionally wherein the first and the second ATUs are in part made of the sequence of polynucleotide of SEQ ID No. 2 or SEQ ID No. 11.

In particular, at least one region separating two heterologous polynucleotides inserted within an intergenic region of the full length antigenomic (+) RNA strand of a non-segmented negative-sense single-stranded RNA virus, especially of the MV, may be derived from the nucleotide sequence of SEQ ID No. 11, wherein each extremity of the sequence corresponds to particular restriction sites, suitable for insertion within a Bacterial Artificial Chromosome (also referenced “BAC” in the present description).

In a particular embodiment, the multiple heterologous polynucleotides (each encoding a heterologous polypeptide issued from a pathogen) inserted within the cDNA molecule encoding a full length antigenomic (+) RNA strand of a non-segmented negative-sense single-stranded RNA virus, especially of a measles virus (MV), are separated from each other by a polynucleotide comprising or consisting of the sequence of SEQ ID No. 2 or SEQ ID No. 11, or a sequence that is issued from SEQ ID No. 2 or SEQ ID No. 11, i.e. shares at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity more preferably at least 98% identity, more preferably at least 99% identity, and most preferentially 100% identity, with SEQ ID No. 2 or SEQ ID No. 11 and preferably has the same length as one of these sequences.

In a particular embodiment, each ATU presents within the cDNA molecule encoding a full length antigenomic (+) RNA strand of a non-segmented negative-sense single-stranded RNA virus, especially of a measles virus (MV), comprises or consists of (i) the sequence of SEQ ID No. 52, SEQ ID No. 53, SEQ ID No. 54, SEQ ID No. 55, SEQ ID No. 56, SEQ ID No. 57, wherein n represents a polynucleotide sequence encoding a heterologous polypeptide; or (ii) a sequence issued from SEQ ID No. 52, SEQ ID No. 53, SEQ ID No. 54, SEQ ID No. 55, SEQ ID No. 56, SEQ ID No. 57, i.e. shares at least 90% identity, more preferably at least 91% identity, more preferably at least 92% identity, more preferably at least 93% identity, more preferably at least 94% identity, more preferably at least 95% identity, more preferably at least 96% identity, more preferably at least 97% 5 identity more preferably at least 98% identity, more preferably at least 99% identity, and most preferentially 100% identity, with SEQ ID No. 52, SEQ ID No. 53, SEQ ID No. 54, SEQ ID No. 55, SEQ ID No. 56, and SEQ ID No. 57, and preferably has the same length as one of these sequences.

In an embodiment of the invention, it is provided a nucleic acid construct wherein a third ATU is localized within the same intergenic region where the first ATU and the second ATU are localized, the third ATU comprising a heterologous polynucleotide operably linked within the third ATU allowing the expression of a heterologous polypeptide encoded by the heterologous polynucleotide wherein said heterologous polypeptide of the third ATU is different from the heterologous polypeptides encoded by the first and the second ATUs, and wherein the heterologous polynucleotides within a single intergenic region of the cDNA are separated from each other by a sequence comprising the polynucleotide of SEQ ID No. 2 or SEQ ID No. 11.

In a particular embodiment, the nucleic acid construct of the invention comprises a cDNA molecule encoding a full length antigenomic (+) RNA strand of a non-segmented negative-sense single-stranded RNA virus, especially of a measles virus (MV), wherein the cDNA molecule further comprises inserted therein a third ATU, wherein the third ATU (i) comprises or consists in the nucleotide sequence of SEQ ID No. 1 wherein the nucleotide sequence from position 79 to position 796 is substituted by a heterologous polynucleotide sequence encoding a selected heterologous polypeptide (said sequence comprising a start codon ATG, since positions 79 to 81 of SEQ ID No. 1 are respectively A, T and G); in particular the third ATU comprises of consists in the same nucleotide sequence as the first ATU or the second ATU but for the coding heterologous polynucleotide that is contains, and (ii) is separated from another closest ATU in the single intergenic region of the cDNA by the polynucleotide of sequence SEQ ID No. 2 or SEQ ID No. 11.

In a particular embodiment, the nucleic acid construct of the invention comprises a cDNA molecule encoding a full length antigenomic (+) RNA strand of a non-segmented negative-sense single-stranded RNA virus, especially of a measles virus (MV), wherein the cDNA molecule further comprises inserted therein a third ATU, wherein the third ATU (i) comprises or consists from its 5′ end towards its 3′ end in the nucleotide sequence of SEQ ID No. 16 or SEQ ID No. 19 or SEQ ID No. 20 SEQ ID No. 20 or SEQ ID No. 40 or SEQ ID No. 41 or SEQ ID No. 42 or SEQ ID No. 43 or SEQ ID No. 44 or SEQ ID No. 45, heterologous polynucleotide sequence encoding a heterologous polypeptide (without the start codon ATG, since SEQ ID No. 16, SEQ ID No. 19, SEQ ID No. 20, SEQ ID No. 20, SEQ ID No. 40, SEQ ID No. 41, SEQ ID No. 42, SEQ ID No. 43, SEQ ID No. 44 and SEQ ID No. 45 all comprise the start codon ATG at their 3′ end), and the nucleotide sequence of SEQ ID No. 17, or SEQ ID No. 46 or SEQ ID No. 47 or SEQ ID No. 48 or SEQ ID No. 49 or SEQ ID No. 50 or SEQ ID No. 51, in particular the third ATU comprises of consists in the same nucleotide sequence as the first ATU or the second ATU but for the coding heterologous polynucleotide that is contains, and (ii) is separated from another closest ATU in the single intergenic region of the cDNA by the polynucleotide of sequence SEQ ID No. 2 or SEQ ID No. 11.

The nucleic acid construct according to the invention comprises ATUs (a first ATU and a second ATU and when present a third ATU) with a heterologous polynucleotide that encodes a heterologous polypeptide that is different from the heterologous polypeptide encoded by the other ATUs. In other words, each heterologous polynucleotide present within the nucleic acid construct differs from the other heterologous polynucleotide(s) inserted within the same nucleic acid construct, and hence each ATU encodes a particular polypeptide different from the polypeptide(s) encoded by other ATUs. In a more particular embodiment of the invention, the nucleic acid construct of the invention comprises a plurality of heterologous polynucleotides, each inserted within an ATU, each heterologous polynucleotide encoding a heterologous polypeptide different from another heterologous polypeptide encoded by another heterologous polynucleotide.

In an embodiment of the invention, the nucleic acid construct may comprise an additional ATU(s) localized in a different intergenic region of the cDNA molecule encoding a full length antigenomic (+) RNA strand of a non-segmented negative-sense single-stranded RNA virus, especially of a measles virus (MV). Such an embodiment is for example illustrated for a measles virus on FIGS. 2D, 2E, 2F and 2G wherein at least one additional ATU is present within the nucleic acid construct in a different intergenic region that the first ATU and the second ATU. In a particular embodiment, the additional ATU comprises or consists in the nucleotide sequence of SEQ ID No. 1 wherein the nucleotide sequence from position 79 to position 796 is substituted by a heterologous polynucleotide sequence encoding at least one heterologous polypeptide (said sequence comprising an start codon ATG, since positions 79 to 81 of SEQ ID No. 1 are respectively A, T and G). In a particular embodiment, the nucleic acid construct of the invention comprises a cDNA molecule encoding a full length antigenomic (+) RNA strand of a non-segmented negative-sense single-stranded RNA virus, especially of a measles virus (MV), wherein the cDNA molecule further comprises inserted therein an additional ATU localized within a different intergenic region than the first and the second ATUs, wherein the additional ATU (i) comprises or consists from its 5′ end towards its 3′ end in the nucleotide sequence of SEQ ID No. 16 or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45, heterologous polynucleotide sequence encoding a heterologous polypeptide (without the start codon ATG which is provided by the nucleotide sequence of SEQ ID No: 16 or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45), and the nucleotide sequence of SEQ ID No. 17 or SEQ ID No. 46 or SEQ ID No. 47 or SEQ ID No. 48 or SEQ ID No. 49 or SEQ ID No. 50 or SEQ ID No. 51, in particular the additional ATU comprises of consists in the same nucleotide sequence as the first ATU or the second ATU but for the coding heterologous polynucleotide that is contains.

Heterologous Polynucleotide Inserted within the Nucleic Acid Construct of the Invention and Encoded Heterologous Polypeptide

Each heterologous polynucleotide localized within an ATU encodes one heterologous polypeptide or a plurality of heterologous polypeptides issued or derived from one or several pathogens, preferably from one pathogen. In a particular embodiment, each ATU comprises a heterologous polynucleotide issued from a pathogen which is a different pathogen from the one(s) providing the other heterologous polynucleotides inserted in another ATU. Heterologous polypeptides may also comprise different antigens issued from a single pathogen, or may comprise the same antigen repeated several times in a single ATU. Alternatively, for example when the production of a fusion protein comprising antigens issued from a single species or form a plurality of different species may lead to a proper immune response against the antigens present in the fusion protein, the heterologous polynucleotide encoding at least one heterologous polypeptide issued or derived from a single pathogen may result from the fusion of several polynucleotides, each encoding a particular heterologous polypeptide, e.g., a particular protein, antigen or an antigenic fragment thereof, of a pathogen. For example, the heterologous polynucleotide may result from the fusion of heterologous polynucleotides each encoding a single heterologous polypeptide of an antigen of a pathogen and such fusion within the nucleic acid construct may be achieved by a linker sequence, in particular a linker sequence corresponding to an intergenic region, or as a result of cloning or insertion in a particular ATU sequence(s). A linker sequence is well known in the art and can be a short nucleotide sequence comprising or consisting in a regulatory sequence of the measles virus.

Accordingly, within each ATU comprising a heterologous polynucleotide, the heterologous polynucleotide may encode a single heterologous polypeptide, two different heterologous polypeptides, two identical heterologous polypeptides, three different heterologous polypeptides, two identical heterologous polypeptides and another heterologous polypeptide, four different heterologous polypeptides, two identical heterologous polypeptides and two others identical heterologous polypeptides, two identical heterologous polypeptides and two others different heterologous polypeptides, three identical heterologous polypeptides and another different heterologous polypeptide. Any one of these heterologous polynucleotides encoding at least two (identical or different) heterologous polypeptides may be issued from the fusion of several heterologous polynucleotides, or prepared using steps of synthesis of nucleic acid fragments or polymerization from a template, including by PCR. Alternatively, any one of these heterologous polynucleotides may be a cDNA issued from the genomic RNA of at least one pathogen, after retrotranscription, said cDNA being either the full genomic cDNA or a fragment thereof, and encoding a polypeptide of the pathogen. The heterologous polynucleotides contained in the at least one ATUs enable expression of heterologous polypeptides as functional separate polypeptides rather than fusion polypeptides.

In an embodiment, the heterologous polypeptides encoded by the heterologous polynucleotides inserted within the ATUs are immunogenic polypeptides originating from or derived from at least one pathogen as defined herein, in particular pathogens infecting human, in particular from at least one of the following viruses: measles virus, Chikungunya virus, Zika Virus, West Nile Virus, Dengue virus, SARS, MERS, coronavirus, in particular SARS-Cov-2, Influenza virus, Ebola virus, Nipas virus, HIV or from a non-viral pathogen such as a parasite including Mycobacterium tuberculosis and Plasmodium species causing malaria, in particular those infecting humans:P. vivax, P. falciparum, P. malariae, P. ovale, and P. knowlesi; in a preferred embodiment, at least one heterologous polypeptide is issued from one of the following viruses: the Chikungunya virus, the West Nile virus, the Zika virus, the SARS virus, the coronavirus, in particular SARS-CoV-2, and/or the HIV. In particular, the pathogen from which the heterologous polypeptide encoded within the genome of the cDNA molecule encoding a full length antigenomic (+) RNA strand of a non-segmented negative-sense single-stranded RNA virus (especially MV) of the invention is issued is a virus, in particular a virus infecting human, especially a virus selected from the list consisting of Chikungunya virus, the West Nile virus, the Zika virus, the SARS virus, the coronavirus, in particular SARS-CoV-2, and/or the HIV. A heterologous polypeptide encoded by the recombinant infectious virus expressed from the nucleic acid construct of the invention may also be the C, E2 and E1 proteins of the Chikungunya virus, the sE protein of the West Nile virus, the prME protein of the Zika virus, the S and M proteins of the SARS (Severe acute respiratory syndrome caused by the SARS Coronavirus), the S and M proteins of the MERS virus (Middle East respiratory syndrome caused by the MERS-Coronavirus) and/or the S and M proteins of the SARS-Cov-2 coronavirus, responsible for the Covid-19 and optionally wherein the heterologous polynucleotides originate or derive from different virus types.

In an alternative embodiment of the invention, a heterologous polypeptide encoded by a heterologous polynucleotide may be a fusion protein as defined in the present description, in particular when the heterologous polypeptide comprises several antigens issued or derived from a single species. In a preferred embodiment of the invention, each heterologous polynucleotide contained in the ATUs enable expression of a heterologous polypeptide comprising antigens issued or derived from a single species.

An example is illustrated on FIG. 11. The amino acid sequences of SEQ ID No. 13, SEQ ID No. 14 and SEQ ID No. 15 are issued from respectively the C, E2 and E1 proteins of the Chikungunya virus (SEQ ID No. 13), the sE protein of the West Nile virus (SEQ ID No. 14), and/or the prME protein of the Zika virus (SEQ ID No. 15) and are encoded by the polynucleotide localized between the nucleotide at position 3532 and the nucleotide at position 11037 in SEQ ID No. 12. Such a nucleic acid construct is furthermore illustrated on FIG. 13, and alternate versions are illustrated on FIG. 16 and FIG. 17, in combination with a BAC plasmid. The present invention encompasses other constructs, wherein the heterologous polynucleotides localized between positions 3532 and 11037 of the polynucleotide of sequence SEQ ID No. 12 comprises another heterologous polynucleotide encoding other immunogenic heterologous polypeptides as defined here above.

More particularly, the heterologous polypeptide encoding the C, E2 and E1 proteins of the Chikungunya virus may comprise or consist of the heterologous polypeptide of SEQ ID No. 13.

More particularly, the heterologous polypeptide encoding the sE protein of the West Nile virus may comprise or consist of the heterologous polypeptide of SEQ ID No. 14.

More particularly, the heterologous polypeptide encoding the prME protein of the Zika virus may comprise or consist of the heterologous polypeptide of SEQ ID No. 15.

In an embodiment of the invention, illustrated on FIG. 12, the heterologous polypeptides encoded by the heterologous polynucleotides inserted within the ATUs are immunogenic heterologous polypeptides of amino acid sequence of SEQ ID No. 13 for C, E2 and E1 proteins of the Chikungunya virus in one ATU, SEQ ID No. 14 for the sE protein of the West Nile virus in another ATU, SEQ No. 15 for the prME protein of the Zika virus in another ATU. Each heterologous polynucleotide encoding a particular immunogenic polypeptide may be separated from the other heterologous polynucleotide(s) by a sequence comprising or consisting of the polynucleotide of SEQ ID No. 2 or SEQ ID No. 11.

In a particular embodiment of the invention, the nucleic acid construct of the invention comprises the polynucleotide of SEQ ID No. 4, which comprises within three distinct ATUs the immunogenic polypeptides C, E2 and E1 proteins of the Chikungunya virus in the first ATU, the sE protein of the West Nile virus in the second ATU and the prME protein of the Zika virus in the third ATU. The polynucleotide of SEQ ID No. 4 may be inserted within any construct disclosed in the present description, in particular within the MV genome of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, preferably the Schwarz strain, the AIK-C strain and the Zagreb strain, in particular the Schwarz strain of SEQ ID No. 62, the AIK-C strain of SEQ ID No. 58 and the Zagreb strain of SEQ ID No. 59. The polynucleotide of SEQ ID No. 4 may be inserted within any intergenic region of the MV genome, more particularly (i) between the P gene and the M gene of the measles virus in the cDNA molecule or (ii) between the H gene and the L gene of the measles virus in the cDNA molecule or (iii) between the N gene of the measles virus and the T7RNA polymerase promoter in the cDNA molecule. In a particular embodiment of the invention, the nucleic acid construct of the invention comprises within the intergenic region localized between the P gene and the M gene of the measles virus a heterologous polynucleotide comprising of consisting of the polynucleotide of SEQ ID No. 4.

These nucleic acid constructs comprise encoded in three distinct ATUs the immunogenic polypeptides C, E2 and E1 proteins of the Chikungunya virus in the first ATU, the sE protein of the West Nile virus in the second ATU and the prME protein of the Zika virus in the third ATU, the first and the second polynucleotides encoding respectively the immunogenic polypeptides C, E2 and E1 proteins of the Chikungunya virus and the sE protein of the West Nile virus being separated from each other by a polynucleotide of SEQ ID No. 2 or SEQ ID No. 11, and the second and the third polynucleotides respectively encoding sE protein of the West Nile virus and the prME protein of the Zika virus being separated from each other by a polynucleotide of SEQ ID No. 2 or SEQ ID No. 11. In particular the ATU(s) is (are) built as the sequence of SEQ ID No. 16 (or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45)-*-SEQ ID No.17 or SEQ ID No. 46 or SEQ ID No. 47 or SEQ ID No. 48 or SEQ ID No. 49 or SEQ ID No. 50 or SEQ ID No. 51 wherein “*” represents one of the above heterologous polynucleotides (without the start codon ATG provided by SEQ ID No. 16, SEQ ID No. 19 or SEQ ID No. 20 and SEQ ID No. 40 to SEQ ID No. 45; a stop codon may or may not be present within the “*” sequence, since SEQ ID No. 17 and SEQ ID No. 46 to SEQ ID No. 51 comprise a stop codon, the presence of an extra STOP codon (or two extra stop codons may be required in order to respect the prescriptions of the rule of 6 as explained here above).

In a particular embodiment, the first ATU and the second ATU comprise or consist of different heterologous polynucleotides that are selected among polynucleotides encoding heterologous polypeptides comprising or consisting in amino acid sequences of SEQ ID No. 13 and SEQ ID No. 14, or selected among heterologous polynucleotides encoding heterologous polypeptides comprising or consisting of amino acid sequences of SEQ ID No. 13 and SEQ ID No. 15, and wherein the first heterologous polynucleotide and the second heterologous polynucleotide are separated by a spacer polynucleotide comprising or consisting in the nucleotide sequence of SEQ ID No. 2 or SEQ ID No. 11. In particular the ATU(s) is (are) built as the sequence of SEQ ID No. 16 (or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45)-*-SEQ ID No. 17 or SEQ ID No. 46 or SEQ ID No. 47 or SEQ ID No. 48 or SEQ ID No. 49 or SEQ ID No. 50 or SEQ ID No. 51 wherein “*” represents one of the above heterologous polynucleotides encoding a polypeptide of SEQ ID No. 13 or SEQ ID No. 15 (the 5′ M amino acid of SEQ ID No. 13 or 15 being encoded by the ATG codon provided by SEQ ID No. 16 or SEQ ID No. 19 or SQ ID No. 20).

In a particular embodiment of the invention, each ATU presents within the cDNA molecule encoding a full length antigenomic (+) RNA strand of a non-segmented negative-sense single-stranded RNA virus, especially of a measles virus (MV), comprises or consists of (i) the sequence of SEQ ID No. 52, SEQ ID No. 53, SEQ ID No. 54, SEQ ID No. 55, SEQ ID No. 56, SEQ ID No. 57, wherein n represents a heterologous polynucleotide encoding a polypeptide of SEQ ID No. 13, SEQ ID No. 14 or SEQ ID No. 15.

In a particular embodiment of the invention, the recombinant nucleic acid molecule or the nucleic acid construct encoding the antigenomic RNA of the virus recombined with the heterologous polynucleotide, which is defined herein is further modified, i.e., comprises additional nucleotide sequences or motifs.

Nucleic Acid Construct Comprising a Measles Virus cDNA Sequence

In an embodiment of the invention, wherein the cDNA molecule encodes a full length antigenomic (+) RNA strand of a measles virus, particular attenuated strains of the measles virus can be used to implement the present invention. Accordingly, in a preferred embodiment of the invention, the non-segmented negative-sense single-stranded RNA virus is a measles virus (MV). In a more particular embodiment, the non-segmented negative-sense single-stranded RNA virus is a measles virus (MV) originates from an attenuated strain.

In a preferred embodiment of the invention, the measles virus is an attenuated virus strain. An “attenuated strain” of measles virus is defined as a strain that is avirulent or less virulent than the parent strain in the same host, while maintaining immunogenicity and possibly adjuvanticity when administered in a host i.e., preserving immunodominant T and B cell epitopes and possibly the adjuvanticity such as the induction of T cell costimulatory proteins or the cytokine IL-12.

An attenuated strain of a MV accordingly refers to a strain which has been serially passaged on selected cells and, possibly, adapted to other cells to produce seed strains suitable for the preparation of vaccine strains, harboring a stable genome which would not allow reversion to pathogenicity nor integration in host chromosomes. As a particular “attenuated strain”, an approved strain for a vaccine is an attenuated strain suitable for the invention when it meets the criteria defined by the FDA (US Food and Drug Administration) i.e., it meets safety, efficacy, quality and reproducibility criteria, after rigorous reviews of laboratory and clinical data (www.fda.gov/cber/vaccine/vacappr.htm).

In a more particular embodiment of the invention, the MV is originating from an attenuated virus strain selected from the group consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, preferably the Schwarz strain, the Zagreb strain and the AIK-C strain in particular the Schwarz strain of SEQ ID No. 62, the AIK-C strain of SEQ ID No. 58 and the Zagreb strain of SEQ ID No. 59. All these strains have been described in the prior art and access to them is provided in particular as commercial vaccines.

In a preferred embodiment, the nucleic acid construct or the recombinant nucleic acid molecule encoding the antigenomic RNA strand of a non-segmented negative-sense single-stranded RNA virus, especially of the measles virus, recombined with the heterologous polynucleotide according to the invention further comprises, at its 5′-end, adjacent to the first nucleotide of the nucleotide sequence encoding the full-length antigenomic (+) RNA strand of non-segmented negative-sense single-stranded RNA virus, especially of the measles virus, a GGG motif followed by a hammerhead ribozyme sequence and also comprises, at its 3′-end, adjacent to the last nucleotide of said nucleotide sequence encoding the full length anti-genomic (+) RNA strand, the sequence of a ribozyme. The Hepatitis delta virus ribozyme (6) is advantageously hence provided at the 3′-end, adjacent to the last nucleotide of said nucleotide sequence encoding the full length anti-genomic (+) RNA strand.

The GGG motif placed at the 5′ end, adjacent to the first nucleotide of the above coding sequence improves the efficiency of the transcription of said cDNA coding sequence. As a requirement for the proper assembly of measles virus particles is the fact that the cDNA encoding the antigenomic (+) RNA of the nucleic acid construct of the invention complies with the rule of six, when the GGG motif is added, a ribozyme is also added at the 5′ end of the coding sequence of the cDNA, 3′ from the GGG motif, in order to enable cleavage of the transcript at the first coding nucleotide of the full-length antigenomic (+) RNA strand of MV.

In a particular embodiment of the invention, in order to prepare the nucleic acid construct of the invention, the preparation of a cDNA molecule encoding the full-length antigenomic (+) RNA of a MV disclosed in the prior art is achieved by known methods. Said cDNA provides especially the genome vector when it is inserted in a vector such as a plasmid.

In an embodiment of the invention, the number of consecutive nucleotides in the nucleic acid construct is a multiple of six and/or the number of consecutive nucleotides in the recombinant cDNA molecule is a multiple of six (specified as “the rule of six”). In a particular embodiment of the invention, the nucleic acid construct comprising the cDNA encoding the nucleotides of the full-length infectious antigenomic (+) RNA strand of a non-segmented negative-sense single-stranded RNA virus, especially of a measles virus (MV), but without the operatively cloned heterologous polynucleotide complies with the rule of six (6) of the measles virus genome and the polypeptide encoding a heterologous polypeptide complies with the rule of six.

A particular cDNA molecule suitable for the preparation of the nucleic acid construct of the invention is the one obtained using the Schwarz strain of MV. Accordingly, the cDNA coding for the antigenome of the measles virus used within the present invention may be obtained as disclosed in WO2004/000876 or may be obtained from plasmid pTM-MVSchw deposited by Institut Pasteur at the Collection Nationale de Culture de Microorganismes (CNCM), 28 rue du Dr Roux, 75724 Paris Cedex 15, France, under No 1-2889 on Jun. 12, 2002, the sequence of which is disclosed in WO2004/000876 incorporated herein by reference. The plasmid pTM-MVSchw has been obtained from a Bluescript plasmid and comprises the polynucleotide coding for the full-length measles virus (+) RNA strand of the Schwarz strain placed under the control of the promoter of the T7 RNA polymerase. It has 18967 nucleotides and a sequence represented as SEQ ID NO. 5. cDNA molecules (also designated cDNA of the measles virus or MV cDNA for convenience) from other MV strains may be similarly obtained starting from the nucleic acid purified from viral particles of attenuated MV such as those described herein.

The cDNA coding for the antigenome of the measles virus used within the present invention may also be obtained from plasmid pTM2-MVSchw-gfp deposited by Institut Pasteur at the Collection Nationale de Culture de Microorganismes (CNCM), 28 rue du Dr Roux, 75724 Paris Cedex 15, France, under No 1-2890 on Jun. 12, 2002. It has 19795 nucleotides and a sequence represented as SEQ ID NO. 6. This plasmid contains the sequence encoding the eGFP marker that may be deleted.

The organization of the genome of measles viruses and their replication and transcription process have been fully identified in the prior art and are especially disclosed in Horikami S. M. and Moyer S. A. (Curr. Top. Microbiol. Immunol. (1995) 191, 35-50 or in Combredet C. et al (Journal of Virology, November 2003, p11546-11554) for the Schwarz vaccination strain of the virus or for broadly considered negative-sense RNA viruses, in Neumann G. et al (Journal of General Virology (2002) 83, 2635-2662).

The “rule of six” is expressed in the fact that the total number of nucleotides present in a nucleic acid representing the MV (+) strand RNA genome or in the nucleic acid constructs comprising the same is a multiple of six. The “rule of six” has been acknowledged in the state of the art as a requirement regarding the total number of nucleotides in the genome of the measles virus, which enables efficient or optimized replication of the MV genomic RNA. In the embodiments of the present invention defining a nucleic acid construct that meets the rule of six, said rule applies to the nucleic acid construct specifying the cDNA encoding the full-length MV (+) strand RNA genome. In this regard, the rule of six applies individually to the cDNA encoding the nucleotide sequence of the full-length infectious antigenomic (+) RNA strand of the measles virus, and/or to the nucleic acid construct encompassing the heterologous polynucleotides cloned into ATU(s) in said cDNA and encoding the heterologous polypeptide(s) of pathogen(s).

Nucleic Acid Construct Comprising a Bacterial Artificial Chromosome (BAC)

In a particular embodiment of the invention, It is provided a recombinant nucleic acid construct comprising a Bacterial Artificial Chromosome comprising a cDNA molecule encoding a full length antigenomic (+) RNA strand of a measles virus (MeV or MV) inserted therein; wherein the cDNA molecule comprises at least one additional transcription unit (ATU), the at least one additional transcription units (ATUs) being localized within an intergenic region of the cDNA, the at least one ATU comprising (i) a heterologous polynucleotide operably linked to enable the expression of a heterologous polypeptide encoded by the heterologous polynucleotide, or (ii) an insertion site suitable for the functional insertion of a heterologous polynucleotide.

A nucleic acid construct comprising a BAC plasmid is for example illustrated on FIGS. 5 and 6, for a measles virus, wherein the cDNA molecule is illustrated inserted within a bacterial artificial chromosome plasmid, the pSMART-BAC, while several heterologous polynucleotides encoding antigen(s) from a pathogen or from a plurality of pathogens are or may be inserted within a plurality of ATUs. A nucleic acid construct of the invention may be prepared using steps of synthesis of nucleic acid fragments or polymerization from a template, including by PCR. The heterologous polynucleotide(s) and nucleic acid construct of the invention may rather be prepared in accordance with any known method in the art and in particular may be cloned, obtained by polymerization especially using PCR methods or may be synthesized.

In a particular embodiment, the recombinant nucleic acid construct as disclosed herein in all embodiments is operably inserted within a recombinant Bacterial Artificial Chromosome plasmid, and the cDNA molecule and the at least one ATUs are defined in accordance with any embodiments disclosed herein.

The cDNA molecule encoding a full length antigenomic (+) RNA strand of a virus, in particular MV, may be inserted within the BAC plasmid throughout the use of restriction enzymes which recognize a restriction site on the BAC plasmid on one hand, and on the cDNA molecule encoding a full length antigenomic (+) RNA strand of a virus on the other hand so as to functionally insert (i.e. in a suitable manner for the expression of the genes of the virus and the expression of the heterologous polypeptide encoded by the heterologous polynucleotide inserted within the ATUs localized within the cDNA molecule encoding a full length antigenomic (+) RNA strand of a virus). The BAC plasmid may be any kind of bacterial artificial chromosome plasmid, in particular the pSMART BAC plasmid and its derivatives, pEZ-BAC plasmid, pBeloBACllplasmid, pBACe3.6, pBAC/OriV, pBAC-RT, pHA1, pHA2, pTARBAC2 and its derivatives, and the pBAC contruct inserted in Transmissible gastroenteritis coronavirus (TGEV), as defined here above. In a preferred embodiment, the bacterial artificial chromosome plasmid is selected from the group consisting of pSMART-BAC, pEZ-BAC and pBeloBAC11. In a more preferred embodiment of the invention, the bacterial artificial chromosome plasmid is the pSMART-BAC plasmid, or is issued from the pSMART-BAC plasmid

In a particular embodiment of the invention, the BAC is devoid of a T7 promoter, when the expression of at least one gene of the virus encoded by the cDNA of its full length antigenomic (+) RNA strand or the heterologous polypeptide encoded by the heterologous polynucleotide inserted within the ATUs, is under control of a T7 promoter. In a particular embodiment of the invention, at least one expression control sequence of the gene of the virus encoded by the cDNA of its full length antigenomic (+) RNA strand or the heterologous polypeptide encoded by the heterologous polynucleotide inserted within the ATUs comprises the T7 promoter and T7 terminator sequences. These sequences are respectively located 5′ and 3′ of the coding sequence for the full length antigenomic (+) RNA strand of the virus and from the adjacent sequences around this coding sequence. Accordingly, in a particular embodiment the nucleic acid construct of the invention comprises these additional control sequences.

The BAC plasmid with the cDNA molecule encoding a full length antigenomic (+) RNA strand of a non-segmented negative-sense single-stranded RNA virus, especially of a measles virus (MV), may correspond to any recombination of a BAC as described herein and any cDNA molecule encoding a full length antigenomic (+) RNA strand of a non-segmented negative-sense single-stranded RNA virus as described herein, in particular any recombination of a cDNA encoding MV genome with heterologous polynucleotides inserted within ATUs allowing expression of different polypeptides issued from pathogens.

In a particular embodiment of the inventions, the BAC plasmid may be issued or derived from a BAC of SEQ ID No. 3, SEQ ID. 7: SEQ ID No. 8, SEQ ID No. 60 or SEQ ID No. 61, SEQ ID No. 12, SEQ ID No. 63 or SEQ ID No. 64 wherein the heterologous polynucleotides inserted within the cDNA molecule encoding a full length antigenomic (+) RNA strand of the measles virus are substituted by other heterologous polynucleotide sequences encoding immunogenic heterologous polypeptides as defined herein. More particularly, the polynucleotide localized between NotI restriction sites of SEQ ID No. 3, or between NotI restriction sites of SEQ ID No. 7, or between NotI restriction sites of SEQ ID No. 8 or SEQ ID No. 60 or SEQ ID No. 61, or between NotI restriction sites of SEQ ID No. 12, or between NotI restriction sites of SEQ ID No. 63, or between NotI restriction sites of SEQ ID No. 64, may be substituted by any other heterologous polynucleotide sequence(s) encoding immunogenic heterologous polypeptides as defined herein especially according to the specific embodiments disclosed. A NotI restriction site is a nucleotide sequence recognized and cut by the NotI restriction enzyme and it corresponds to the nucleotide sequence of SEQ ID No. 18 (5′ GCGGCCGC 3′).

In a particular embodiment of the invention, it is provided a nucleic acid construct as described herein or a recombinant BAC plasmid as described herein, wherein a third ATU is localized within the same intergenic region where the first ATU and the second ATU are localized, the third ATU comprising a heterologous polynucleotide operably linked within the third ATU allowing the expression of a heterologous polypeptide encoded by the heterologous polynucleotide wherein the heterologous polypeptide is different form the heterologous polypeptides encoded by the heterologous polynucleotides of the other ATUs, and optionally wherein the heterologous polypeptide(s) encoded by each ATU originate from different virus types. In other particular embodiments, the nucleic acid construct comprises a Measles virus, a BAC plasmid, and three ATUs, and the three ATUs are localized within the same intergenic region of the Measles virus in the cDNA molecule, in particular the three ATUs are localized between the P gene and the M gene of the measles virus in the cDNA molecule; or between the H gene and the L gene of the measles virus in the cDNA molecule, or between the N gene and the T7RNA polymerase promoter.

In another particular embodiment of the invention, it is provided a nucleic acid construct as described herein, in particular a recombinant BAC plasmid as described herein, wherein the intergenic region localized between the P gene and the M gene of the measles virus in the cDNA molecule comprises all the ATUs, and wherein the heterologous polynucleotides inserted in the intergenic region are separated by a spacer polynucleotide comprising or consisting of SEQ ID No. 2 or SEQ ID No. 11.

In another particular embodiment of the invention, it is provided a nucleic acid construct as described herein in particular a recombinant BAC plasmid as described herein, comprising a third ATU, wherein the first ATU, the second ATU and the third ATU comprise(s) or consist(s) of different polynucleotides encoding heterologous polypeptides comprising or consisting in amino acid sequences that are selected among SEQ ID No. 13, SEQ ID No. 14 and SEQ ID No. 15, and wherein the heterologous polynucleotide encoding the heterologous polypeptides are separated by a spacer polynucleotide comprising or consisting in the nucleotide sequence of SEQ ID No. 2 or SEQ ID No. 11. In a particular embodiment, these heterologous polypeptides comprising or consisting in amino acid sequences that are selected among SEQ ID No. 13, SEQ ID No. 14 and SEQ ID No. 15 are all inserted within ATUs localized between the P gene and the M gene of the measles virus in the cDNA molecule.

Method for Producing a Vaccine

The present invention also concerns a method for producing a vaccine based on recombinant infectious viral particles and/or virus-like particles.

To this end, the invention also relates to a process for rescuing recombinant infectious non-segmented negative-sense single-stranded RNA virus, especially recombinant infectious Measles virus, expressing at least one, in particular at least two, heterologous polypeptides or immunogenic fragments thereof or antigenic fragments thereof expressed from at least one, in particular by at least two, heterologous polynucleotide inserted within an ATU localized within a cDNA molecule encoding a full length antigenomic (+) RNA strand of a measles virus (MV), said heterologous polypeptides being in particular issued from a single or a plurality of pathogens as already defined herein, and comprising:
(a) In cells, in particular HEK293 helper cells, stably expressing T7 RNA polymerase and measles virus N and P proteins and/or transfected with an expression vector encoding T7 RNA polymerase and/or transfected with an expression vector encoding measles virus N and P proteins, transfecting any nucleic acid construct according to any embodiment described herein;
(b) Maintaining the transfected cells of step (a) in conditions suitable for the production of recombinant virus and/or virus like particles;
(c) Infecting cells, in particular Vero cells, in conditions enabling the propagation of the recombinant infectious virus by co-cultivating these cells with the cells issued from step (b); in particular at 32° C.;
(d) Harvesting the recombinant infectious virus and/or virus like particles, in particular at 32° C.

In a particular embodiment, expression is characterized by being maintained over at least 12 passages of the rescued recombinant infectious negative-sense single-stranded RNA virus when amplified on Vero cells, especially of the amplified recombinant infectious measles virus.

According to a particular embodiment, the harvesting of the recombinant virus, in particular recombinant Measles virus, expressing at least one polypeptide of the pathogen or an immunogenic fragment or an antigenic comprises measles viruses expressing the C, E2 and E1 proteins of the Chikungunya virus, the sE protein of the West Nile virus, and/or the prME protein of the Zika virus, and are in particular polypeptides of amino acid sequence of SEQ ID No. 13 for C, E2 and E1 proteins of the Chikungunya virus, SEQ ID No. 14 for the sE protein of the West Nile virus, SEQ ID No. 15 for the prME protein of the Zika virus.

0 According to a particular embodiment of said process, the transfer vector plasmid has the sequence of SEQ ID NO. 12.

According to a particular embodiment of said process, the transfer vector plasmid has the sequence of SEQ ID NO. 63.

According to a particular embodiment of said process, the transfer vector plasmid has the sequence of SEQ ID NO. 64.

According to a particular embodiment, the harvesting step is performed at 32° C.

According to a particular embodiment, the infection step is performed at 32° C.

According to a particular embodiment, the harvesting and the infection steps are both performed at 32° C. The co-cultivation step between infection and harvesting may be performed at any temperature ranging from 32° C. to 37° C., both extreme temperatures of 32° C. and 37° C. being explicitly included within the range, more preferably either at 37° C. or at 32° C., and most preferably at a temperature of 32° C.

As used herein, the term “recombining” applied to cells means introducing at least one polynucleotide into a cell, for example under the form of a vector, said polynucleotide integrating (entirely or partially) or not integrating into the cell genome (such as defined above).

According to a particular embodiment, recombination can be obtained with a first polynucleotide, which is the nucleic acid construct of the invention. Recombination can also, or alternatively, encompass, introducing a polynucleotide, which is a vector encoding a RNA polymerase large protein (L) of a MV, whose definition, nature and stability of expression has been described herein. In accordance with the invention, the cell or cell lines or a culture of cells stably producing a RNA polymerase, a nucleoprotein (N) of a measles virus and a polymerase cofactor phosphoprotein (P) of a measles virus is a cell or cell line as defined in the present specification or a culture of cells as defined in the present specification, i.e., are also recombinant cells to the extent that they have been modified by the introduction of one or more polynucleotides as defined above. In a particular embodiment of the invention, the cell or cell line or culture of cells, stably producing the RNA polymerase, the N and P proteins, does not produce the L protein of a measles virus or does not stably produce the L protein of a measles virus, e.g., enabling its transitory expression or production.

The production of recombinant infectious replicating virus particles of the invention may involve a transfer of cells transformed as described herein. The term “transfer” as used herein refers to the plating of the recombinant cells onto a different type of cells, and particularly onto monolayers of a different type of cells. These latter cells are competent to sustain both the replication and the production of infectious virus particles, i.e., respectively the formation of infectious viruses inside the cell and possibly the release of these infectious viruses outside of the cells. This transfer results in the co-culture of the recombinant cells of the invention with competent cells as defined in the previous sentence. The above transfer may be an additional, i.e., optional, step when the recombinant cells are not sufficiently efficient virus-producing culture, i.e., when infectious virus particles cannot be efficiently recovered from these recombinant cells. This step is introduced after further recombination of the recombinant cells of the invention with nucleic acid construct of the invention, and optionally a vector comprising a nucleic acid encoding a RNA polymerase large protein (L) of a measles virus.

In a particular embodiment of the invention, a transfer step is required since the recombinant cells, usually chosen for their capacity to be easily recombined are not efficient enough in the sustaining and production of recombinant infectious virus particles. In said embodiment, the cell or cell line or culture of cells of step 1) of the above-defined methods is a recombinant cell or cell line or culture of recombinant cells according to the invention.

Cells suitable for the preparation of the recombinant cells of the invention are prokaryotic or eukaryotic cells, particularly animal or plant cells, and more particularly mammalian cells such as human cells or non-human mammalian cells or avian cells or yeast cells. In a particular embodiment, cells, before recombination of its genome, are isolated from either a primary culture or a cell line. Cells of the invention may be dividing or non-dividing cells.

According to a preferred embodiment, helper cells are derived from human embryonic kidney cell line 293, which cell line 293 is deposited with the ATCC under No. CRL-1573. Particular cell line 293 is the cell line disclosed in the international application WO2008/078198 and referred to in the following examples.

According to another aspect of this process, the cells suitable for passage are CEF cells. CEF cells can be prepared from fertilized chicken eggs as obtained from EARL Morizeau, 8 rue Moulin, 28190 Dangers, France, or from any other producer of fertilized chicken eggs.

The process which is disclosed according to the present invention is used advantageously for the production of infectious replicative MV particles that may be used in an immunogenic composition.

Compositions, Vaccines and Seeds Issued or Derived from a Nucleic Acid Construction According to the Invention

The invention also relates to an immunogenic composition, advantageously a vaccine composition comprising (i) an effective dose of the recombinant measles virus according to the invention, and/or of the recombinant VLPs according to the invention and (ii) a pharmaceutically acceptable vehicle, wherein the composition or the vaccine elicits a humoral, especially a protective, in particular a neutralizing humoral response and/or a cellular response in an animal host, especially in a human being, in particular after a single immunization, against the polypeptide(s) of the pathogen.

Also encompassed by the present invention, it is provided a seed preparation or a stock preparation of a recombinant infectious non-segmented negative-sense single-stranded RNA virus, especially of a recombinant infectious measles virus that consists in an single virus clone or respectively an amplified single virus clone of a recombinant infectious virus rescued by reverse genetics from any nucleic acid construct described herein in particular from any recombinant BAC plasmid described herein.

It is also provided a vaccine which comprises a clonally selected and amplified recombinant infectious negative-sense single-stranded RNA virus, especially a clonally selected and amplified recombinant infectious measles virus obtainable from the rescue by reverse genetics of any nucleic acid construct described herein in particular from the recombinant BAC plasmid according to any one of the recombinant BAC plasmids described herein.

Also encompassed by the present invention is a recombinant infectious non-segmented negative-sense single-stranded RNA virus, especially a recombinant infectious Measles virus, wherein the genome of said recombinant infectious virus comprises or consists in a nucleic acid construct according to any embodiment described herein.

The invention also relates to an immunogenic or vaccine composition comprising (i) an effective dose of the recombinant virus according to the invention, and/or of the recombinant VLPs according to the invention and (ii) a pharmaceutically acceptable vehicle for use in the prevention or treatment of an infection by a pathogen In a particular embodiment, said composition is for administration to children, adolescents or travelers.

The invention also relates to an immunogenic composition which comprises a clonally selected and amplified recombinant infectious negative-sense single-stranded RNA virus, especially a clonally selected and amplified recombinant infectious measles virus obtainable from the rescue by reverse genetics of any nucleic acid construct according to the present description, or from the recombinant BAC plasmid according to any embodiment described herein.

In a particular embodiment, the immunogenic vaccine or composition of the invention enables stable expression of multiple heterologous polypeptides from any nucleic acid construct according to the present description, or from the recombinant BAC plasmid according to any embodiment described herein, wherein such stable expression is characterized by being maintained over at least 12 passages of the rescued recombinant infectious negative-sense single-stranded RNA virus when amplified on Vero cells, especially of the amplified recombinant infectious measles virus.

Said composition may comprise a suitable vehicle for administration e.g. a pharmaceutically acceptable vehicle to a host, especially a human host and may further comprise but not necessarily adjuvant to enhance immune response in a host. The inventors have indeed shown that the administration of the active ingredients of the invention may elicit an immune response without the need for external adjuvantation.

Such a vaccine composition comprises advantageously active principles (active ingredients) which comprise recombinant infectious replicating virus particles rescued from the vector and constructs as defined herein optionally associated with VLPs comprising the (same) pathogen proteins.

The administration scheme and dosage regime may require a unique administration of a selected dose of the recombinant infectious replicating virus particles. Alternatively, it may require administration of multiple doses.

In a particular embodiment the administration is performed in accordance with a prime-boost regimen. Priming and boosting may be achieved with identical active ingredients consisting of said recombinant infectious replicating virus particles. Alternatively, priming and boosting administration may be achieved with different active ingredients, involving said recombinant infectious replicating virus particles in at least one of the administration stages of the prime-boost administrations.

Considering that the currently known doses for human vaccines are in the range of 10³ to 10⁵ TCID50, a suitable dose of recombinant virus to be administered may be in the range of 0.1 to 10 ng, in particular 0.2 to 6 ng, and possibly as low as 0.2 to 2 ng.

According to a particular embodiment of the invention, the immunogenic or vaccine composition defined herein may also be used for protection against an infection by the measles virus.

The invention also relates to a method for preventing a pathogen virus related disease said method comprising the immunization of a mammalian, especially a human, in particular a child, by the injection, in particular by subcutaneous injection, of recombinant infectious virus according to the invention.

The invention also relates to a combination vaccine which comprises a recombinant measles virus which encodes at least two, in particular three or more than three immunogenic polypeptides originating from or derived from distinct pathogens infecting human, in particular from at least two viruses selected from the group of Chikungunya virus, West Nile virus and Zika virus, wherein the immunogenic polypeptides are expressed as individual polypeptides by the recombinant virus. In a more particular embodiment, the combination vaccine allows the expression of multiple heterologous polypeptides that are obtained from a recombinant virus rescued using any nucleic acid construct according to the invention or the recombinant BAC plasmid according to any embodiment of the invention.

The invention also relates to a method for treating a pathogen virus related disease, said method comprising the immunization of a mammalian, especially a human, in particular a child, by the injection, in particular subcutaneous injection, of recombinant infectious virus according to the invention.

Infectious Ribonucleoprotein Complexes (RNPs)

The invention also relates to infectious ribonucleoprotein complexes (RNPs) also designated ribonucleoproteins, ribonucleocapsids or ribonucleoparticles of the recombinant measles viruses, generated by reverse genetics. The preparation of the RNPs may be achieved using techniques known from the person skilled in the art such as those disclosed in WO 2009/095791.

As defined herein, the term “RNP” refers to a structure containing a replicating recombinant MV ribonucleic acid (RNA) obtained according to the invention and the transcriptase complex formed of MV nucleoprotein (N) phosphoprotein (P) and polymerase (L) auto-assembled to form a structure, in particular a rod-shaped structure.

Accordingly, an object of the present invention is infectious RNPs from recombinant measles virus (MV) expressing heterologous polypeptide(s), obtained using the nucleic acid construct defined herein which comprises a cDNA molecule encoding a full length antigenomic (+) RNA strand of a non-segmented negative-sense single-stranded RNA virus, especially of a measles virus (MV), wherein the cDNA molecule further comprises inserted therein, at least a first additional transcription unit (ATU) and a second additional transcription unit (ATU), wherein the at least first and second additional transcription units (ATUs) are localized within a single intergenic region of the cDNA encoding the antigenomic (+) RNA strand as a single expression cassette, and wherein each ATU comprises a heterologous polynucleotide operably inserted within the ATU allowing the expression of a heterologous polypeptide encoded by the heterologous polynucleotide.

According to a particular embodiment of the invention, the RNPs may be obtained using any of the herein disclosed embodiments of this nucleic acid construct.

The invention thus relates to a Ribonucleoprotein of a recombinant measles virus strain which comprises a RNA genome assembled with the transcriptase complex proteins of the measles virus wherein the genome contains the recombinant full length antigenomic (+) RNA strand of the measles virus (MV), and inserted therein, sequences encoding at least a first additional transcription unit (ATU) and a second additional transcription unit (ATU), wherein the at least first and second additional transcription units (ATUs) are localized within a single intergenic region of the recombinant antigenomic (+) RNA strand as a single expression cassette, and wherein each ATU comprises a heterologous polynucleotide operably inserted within the ATU allowing the expression of a heterologous polypeptide encoded by the heterologous polynucleotide

RNPs according to the invention thus express the heterologous polypeptides encoded by the recombinant cDNA of the invention.

RNPs may be obtained using methods known in the art such as methods carried out in yeast, especially budding yeast as illustrated by Saccharomyces cerevisiae.

In a particular embodiment of the invention, the recombinant host cell, in particular yeast strain, suitable for the expression of infectious non-segmented negative-strand RNA virus Ribonucleocapsids (RNPs), is obtained after being transformed with at least the following expression vector:
(i) At least one genome vector, comprising, as an insert under the control of regulatory expression sequences functional in the host cell, a cloned DNA molecule which comprises a nucleic acid construct of the invention.

In another particular embodiment of the invention, the recombinant host cell, in particular yeast strain, suitable for the expression of infectious recombinant measles virus Ribonucleocapsids (RNPs), is obtained after being transformed with at least the combination of the following expression vectors:
(i) at least one genome vector, comprising, as an insert under the control of regulatory expression sequences functional in the host cell, a cloned DNA molecule which comprises a nucleic acid construct of the invention,
(ii) one or more trans-complementation vectors comprising, under the control of regulatory expression sequences functional in the host cell, nucleotide sequences which enable said vector(s) to collectively express the proteins necessary for the synthesis of the viral transcriptase complex of said recombinant measles virus used in the genome vector, and enable assembly of the ribonucleocapsid (RNPs) of said virus, which are functional in the host cell for the replication and transcription, said vector(s) further optionally comprising, under the control of regulatory expression sequences functional in the host cell, a selectable marker.

In a particular embodiment, the RNA of the MV used to derive the nucleic acid construct is the RNA of a Schwarz strain, an AIKC strain or a Zagreb strain as disclosed herein.

RNPs can be used in an immunogenic composition or may be used as seeds to reproduce the viral particles or virus-like particles of the said recombinant measles virus, in cell culture in conditions that would allow their use as immunogens, preferably as vaccine components.

### SHORT DESCRIPTION OF THE DRAWINGS

These and further aspects of the invention will be explained in greater detail by way of examples and with reference to the accompanying drawings in which:

FIG. 1: Schematic diagram of measles virus and its non-segment negative RNA, genome, structural proteins (N, nucleoprotein; P, phosphoprotein; M, matrix protein; F, fusion protein; H, hemaglutinin; L, Large polymerase), and accessory proteins (C and V protein) in the vector (pTM-MVSchw) containing an additional transcript unit (ATU). T7, T7RNA polymerase promoter; hh, hammerhead ribozyme; δ, hepatitis delta virus (HDV) genome ribozyme; T7t, T7RNA polymerase terminator.

FIG. 2. Schematic illustration of measles virus and its non-segment negative RNA wherein several different ATUs have been incorporated. From A) to G), several embodiments are illustrated where the localization of the ATUs are different. ATU references (e.g. ATU1, ATU2 ATU3, etc.) do not involve a particular order, and/or a correspondence to the ATUs disclosed in prior art, wherein ATU1 is usually localized upstream the N gene of the MV genome, ATU2 is localized between genes P and M of the MV genome, and ATU3 is localized between the H and L genes of the MV genome. The numeric reference associated to each ATU has herein only an illustrative purpose, to clearly illustrate that a plurality of ATUs may be localized between a single intergenic region of the MV genome or between multiple intergenic regions of the MV genome, but these ATUs may have a different structure, or share the same structure as detailed in the description of the invention, and may or may not be equivalent to the ATUs disclosed in prior art.

FIG. 3. pSMART-BACv2 plasmid (Lucigen) genomic map. The vector incorporates controllable genetic elements that allow the single copy BAC to be amplified in vivo; ori2 (oriS) origin of replication, repE gene, and sopABC partition loci. The vector also carries the oriV medium-copy origin of replication for arabinose inducible promoter. The schematic also indicates the targeted features/restriction sites (underlined in red) that are modified by either removal of the region, T7 promoter, or site-directed mutagenesis to abolish the restriction sites, BssHII, SalI and FseI, allowing further insertion of targeted genes into ATU2 on MV vector.

FIG. 4. Modified pSMAC-BAC plasmid map with T7 promoter between SwaI and NotI sites removed and replaced with chimeric intron region amplified from pCI-Neo. The two sites of BssHII, position 3396 and 4540 were site-directed mutated from C3397 to A and G4542 to A while keeping the same encoding amino acid. To allow insertion of genes in the ATU2 and ATU3 simultaneously, SalI (position 6195) and FseI (position 1021) restriction sites were also site-directed mutated from T 6196 to A and G1020 to C.

FIG. 5: A) pSMART-BAC-2.0 (Lucigen) and its modifications to generate pSMAC-BAC. B) pTM-MV map. C) pSMAC-MV map.

FIG. 6. pSMAC-MV plasmid map; blue box indicates pSMAC vector backbone, grey box indicates the antigenome of Schwarz MV with each measles gene shown in light yellow boxes with translation elements in green boxes.

FIG. 7: Introduction of fluorescence genes into ATU2 of pSMAC-MV into BsiWI and BssHII restriction sites of ATU2. A) Intergenic sequence: fragment 3331-3392 of MV Schwarz genomic sequence with single nucleotide change T to A at position 3365 to mutate SalI restriction site was linked to fragment 1735-1802 of MV Schwarz genomic sequence containing the CTT start-stop signal and P promoter. Two restriction sites (6-base and 8-base respectively) were added at the 5′ and 3′ end of the intergenic sequence. The unique BstBI and FseI restriction sites were added to facilitate further insertion of additional genes. B) The encoding sequences of fluorescent GFP and mCherry proteins were added on both sides of the intergenic sequence and the whole fragment was introduced into ATU2 of pSMAC-MV to allow GFP and mCherry expression simultaneously from ATU2.

FIG. 8: pSMAC-MV-GFP-mCherry (pSMAC-MV-GM) plasmid map. The additional fluorescent genes were added at the ATU2 position between measles virus P and M genes. The GFP gene is indicated in green and the mCherry gene in red with the intergenic region (144 bp) between BstBI and FseI sites.

FIG. 9: Average syncytia counts after recovery of MV-GFP-mCherry comparing between pSMAC and pTM vectors. The experiments were performed in triplicate and 3 biological repeats.

FIG. 10: A) Intergenic sequence with possibility of any unique 6- or 8-nucleotide restriction site to facilitate the swapping of genes in the multiple ATU. B) Schematic map of pSMAC-MV with multiple ATUs inserted in ATU2 BsiWI/BssHII enabling 3 additional genes expressed separately in the same position of recombinant virus.

FIG. 11. Schematic diagram of a measles virus according of FIG. 1. The pSMAC-MV vector contains three antigenic sequences from CHIKV, WNV and ZIKV with intergenic sequences in between, and inserted between the P gene and the M gene of the MV genome, the MV being inserted within a BAC, as illustrated in FIG. 1. CHIKV: Chikungunya Virus—WNV: West Nile Virus—ZIKV: Zika Virus ZIKV with intergenic sequences in between them corresponding to SEQ ID No. 2.

FIG. 12. Cloning strategy for pSMAC-MV-CIWIZ plasmid containing 3 immunogens of CHIKV, WNV and ZIKV plus intergenic sequences inserted in ATU2. A) Cloning strategy for the multiple antigens to achieve construct 2 (CHIKV and WNV) and construct 3 (CHIKV and ZIKV). This first step was done using extension PCR. B) Cloning steps to achieve the multivalent construct to ligate the CHIKV fragment to the WNV and ZIKV fragment through Sbfl site, giving the triple antigens construct that can be inserted into ATU2 using restriction enzymes, BsiWI and BssHII.

FIG. 13. Schematic map of pSMAC-MV-CIWIZ (CHIKV-WNV-ZIKV) vector plasmid. CHIKV, WNV and ZIKV genes are represented in orange, blue and green arrows respectively. The intergenic region or ATU 2.1 and 2.2 are indicated in pink boxes. Total size of pSMAC-MV-CIWIZ is 31676 bp.

FIG. 14. Schematic representation of additional constructs with polynucleotides encoding three immunogens of CHIKV, WNV and ZIKV inserted into pSMAC-MVschw into ATU2 and/or ATU1. In construct 1) (referenced pSMAC-2CHIK-3WIZ) CHIKV antigen is encoded by a polynucleotide inserted into ATU2 and WNV and ZIKV antigens are encoded by a polynucleotide inserted into ATU3 wherein the polynucleotide comprises an intergenic region between these two antigens, the intergenic region comprising the end of a first ATU as described herein, and the beginning of a second and subsequent ATU as described herein. In construct 2) (referenced pSMAC3-CIWIZ), all three antigens are encoded by a polynucleotide inserted into ATU3 wherein the polynucleotide comprises an intergenic region between the antigens, each intergenic region comprising the end of a first ATU as described herein, and the beginning of a second and subsequent ATU as described herein.

FIG. 15. Schematic representation of cloning strategy for creating pSMAC with CHIKV gene in the ATU2 and WNV gene and ZIKV gene interspaced by an intergenic region in the ATU3 (i.e. the construct referenced pSMAC-2CHIK-3WIZ in the present description). The required fragments were amplified separately from different plasmids and then reassembled into a SalI-BssHII open pSMAC3-MV vector.

FIG. 16. Schematic representation of pSMAC-MV vector containing three antigenic sequences from CHIKV, WNV and ZIKV with intergenic sequences in between, and inserted into ATU3. This vector has the sequence of SEQ ID No. 64 and is referenced pSMAC3-CIWIZ in the present description.

FIG. 17. Schematic representation of pSMAC-MV vector containing one antigenic sequence from CHIKV inserted into ATU2 and two antigenic sequences from WNV and ZIKV inserted into ATU3. This vector has the sequence of SEQ ID No. 63 and is referenced pSMAC-2CHIK-3WIZ in the present description.

FIG. 18. Western Blot analysis of cell lysates from Vero cells infected with bivalent or trivalent recombinant measles viruses. Control cell lysates were from Vero cells infected with MV-CHIK, MV-WNV and MV-ZIKV separately. For CHIKV antigens, anti-CHIK-E1 antibody detecting the E1 envelope protein (50 KDa) was used. For WNV and ZIKV antigens, the anti-flavivirus envelope protein (clone 4G2) was used to detect WNV secreted envelope protein (50 KDa) and ZIKV surface envelope protein (60 KDa). All the clones with the red stars were positive for the antigens expression and were chosen for amplifying viral stocks and further analysis.

FIG. 19. Growth kinetics of recombinant MV constructs in Vero cells infected at an MOI of 0.1. Cell-associated virus titers are indicated in TCID₅₀/ml. A and B represent two different set of viruses rescued comparing to MV-Schwarz and MVs with single antigen control.

FIG. 20. Homologous prime-boost immunization of IFNAR −/− mice (n=6; or n=4 when a single antigen is encoded within the MV and for MV control). Mice were immunized intraperitoneally with 1×10⁵ TCID50 of the indicated recombinant MV at days 0 and 30. Sera were collected 30 and 51 days after immunization.

FIG. 21. Humoral responses detected in mice sera using specific ELISA to detect antibodies to the three different antigens encoded within constructs according to the invention and listed on the right hand of the figure. Prime/boost vaccination is analysed by measuring antibody responses against A) MV antigens, B) CHIKV-E protein, C) ZIKV-protein or D) WNV-secreted E protein. The data show the reciprocal endpoint dilution titers with each data point representing an individual animal.

FIG. 22. Harvest of MV-GFP grown on Vero CCL-81 cells cultured in VP-SFM medium without serum at 32 or 37° C. Virus was harvested from cells and medium and tittered by the TCID50 method. Different conditions of cell density were tested.

FIG. 23. Harvest of MV-GFP grown on Vero CCL-81 cells cultured in VP-SFM medium without serum at 32° C. Virus was harvested from medium and tittered by the TCID50 method.

FIG. 24. Schematic representation of the MV genome, wherein the promoter sequences and the terminator sequences of the genes of MV are highlighted. Promoter sequences and terminator sequences of (i) the gene N are in red; (ii) the gene P are in green; (iii) the gene M are in orange; (iv) the gene H are in dark blue; (v) of the gene L are in black. These regulatory sequences may be used in the ATUs according to the invention; the promoter sequence of a gene of the MV may be present within an ATU according to the invention, as well as the terminator sequence.

FIG. 25. Examples of ATUs suitable for insertion into the genome of a MV genome. Different ATUs are illustrated and correspond to ATUs designed for the expression of the heterologous polypeptide encoded by the heterologous polynucleotide inserted within each ATU present within the genome of a MV. The “CTT” codons in cyan corresponds to the START/STOP codon of the ATUs; the sequences in purple correspond to the promoter sequences of the ATUs, and are issued from the promoter sequences of the genes of MV; the sequences in orange correspond to restriction sites; the green sequences consists in the START codon and the STOP codon of the heterologous polynucleotides encoding the heterologous polypeptides to be inserted within the ATUs at the “insertion site”; the sequences in red correspond to the terminator sequences of the ATUs, and are issued from the terminator sequences of the genes of MV. (A). ATU with F promoter sequence issued from the promoter region of the gene F of MV and F terminator sequence issued from the terminator region of the gene F of MV; (B) ATU with H promoter sequence issued from the promoter region of the gene H of MV and H terminator sequence issued from the terminator region of the gene H of MV; (C) ATU with L promoter sequence issued from the promoter region of the gene L of MV and L terminator sequence issued from the terminator region of the gene L of MV; (D) ATU with M promoter sequence issued from the promoter region of the gene M of MV and M terminator sequence issued from the terminator region of the gene M of MV; (E) ATU with N promoter sequence issued from the promoter region of the gene N of MV and N terminator sequence issued from the terminator region of the gene N of MV; (F) ATU with P promoter sequence issued from the promoter region of the gene P of MV and M terminator sequence issued from the terminator region of the gene P of MV.

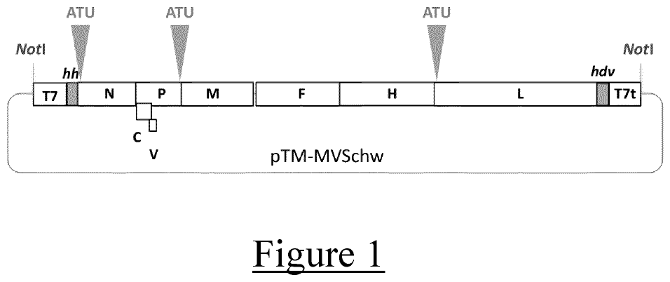

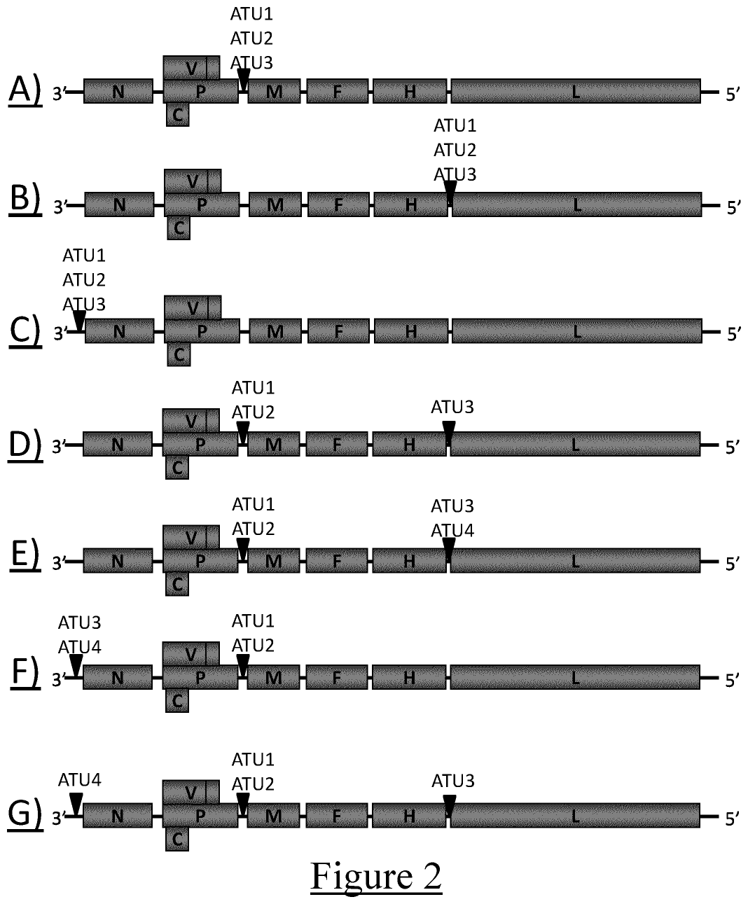

### EXAMPLES OF THE INVENTION

Modification of pSMART-BAC

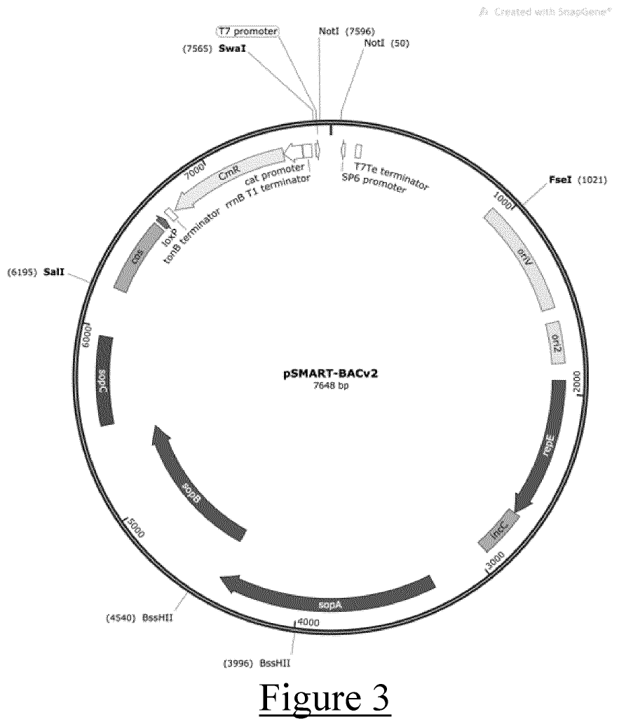

Commercially available pBAC or pSMART-BAC were previously used to clone several large non-segmented RNA viruses²⁴. We used pSMART-BAC (Lucigen pSMART-BAC v2.0) to insert the full-length MV genome into NotI restriction sites (FIG. 3). However, several modifications of the original pSMART-BAC were first needed in order to allow a successful rescue of measles virus. First, the existing T7 promoter on pSMART-BAC needed to be removed to prevent interfering with the expression from the T7 promoter already present upstream of the MV full-length genome. Second, two BssHII sites on pSMART-BAC were mutated by site directed mutagenesis to abolish BssHII enzyme recognition while maintaining the same amino acid sequence to allow the insertion of heterologous sequences in the additional transcription units (ATUs)³. Similarly, the SalI restriction site was also mutated by site directed mutagenesis to allow insertion of the heterologous genes into ATU2 and ATU3 at the same time. Lastly, the FseI restriction site was also mutated to enable the multiple gene insertion into a single ATU. The detailed modification of pSMART-BAC is described in the following:

1. Removing T7 Promoter from the Mutated pSMART-BAC Plasmid

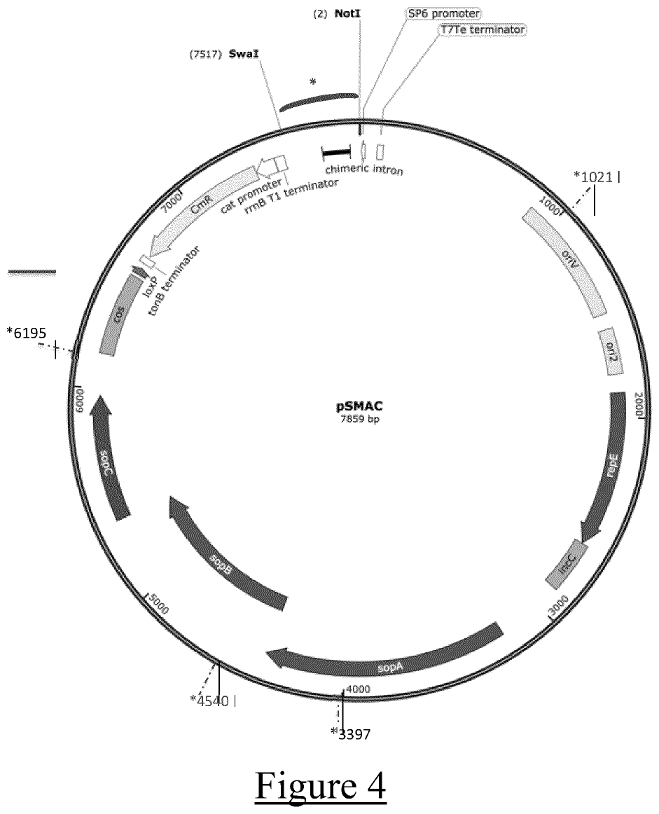

To remove the T7 promoter from pSMART-BAC-GFP plasmid, a 348 bp linker DNA was amplified from pCI-Neo to replace between SwaI and NotI sites (primers are indicated in Table 1). The mutated pSMART-BAC-GFP bacmid and the PCR fragment were digested with SwaI and NotI (Anza restriction system Invitrogen). After ligation, the pSMART-BAC-GFP plasmid mutated in BssHII sites and deleted of its T7 promoter was generated and named pSMAC-BAC (FIG. 4).

TABLE 1
primers used to remove T7 promoter from the
pSMART-BAC-GFP plasmid

| Primer | | |
|---|---|---|
| name | Sequence 5′-3′ | SEQ ID |
| SwaI- | TGATTTAAATTCGTTTAGTGAACCGTCAGATC | 25 |
| Neo | | |
| NotI- | TTGCGGCCGCAGTACTCTAGCCTTAAGAGCTG | 26 |
| Neo | | |

2. Site-Directed Mutagenesis of Two BssHII Sites in the pSMART-BAC-GFP Plasmid

pSMART plasmid contains two BssHII restriction sites, one in the sopA gene at position 3397 and another in the sopB gene at position 4542. Site-directed mutagenesis was carried out by using GeneART Site-Directed Mutagenesis PLUS kit (ThermoFisher Scientific). The primers were designed to replace nucleotide C3397-A and G4542-A and maintain the same in-frame amino acid (Table 2). Primers were mixed in order of MIX1, 2 μl of pSopA-fwd and pSopB-rev for reaction Tube 1, and MIX2, 2 μl of pSopB-fwd and pSopA-rev for reaction Tube 2. Site-directed mutagenesis reactions were prepared according to the manual and aliquot 20 μl for each reaction using Q5 High-Fidelity 2× Master Mix (NEB). The amplification step was performed with optimum extension times for Tube1 and tube 2 at, 3:33 min and 16 sec, respectively. The PCR products were analyzed on the gel electrophoresis with 7 kb and 500 bp for Tube1 and 2, respectively. The excised and purified bands were used for a 20-μl recombination reaction by adding 2 μl of PCR product from Tube 1 and 2, plus the addition of 8 μl DNase-RNase free water and GeneArt 2× Enzyme mix for 10 μl. The recombination reaction was stopped by adding 1 μl 0.5M EDTA. The product of this reaction (3 μl) was used to transform DH5aTH-T1R competent cells according to the manual and transformants were screened onto LB plate containing Chloramphenicol. The positive clones were picked for plasmid amplification and purification. The mutated clones were confirmed by BssHII digestion and sequencing.

TABLE 2
Primers used for site directed mutagenesis to
remove BssHII restriction sites in pSMART

| Primer | | |
|---|---|---|
| name | Sequence 5′-3′ | SEQ ID |
| pSopB- | CATTACTCCTACGCGAGCAATTAACGAATCC | 21 |
| fwd | | |
| pSopB- | GGATTCGTTAATTGCTGCCGTAGGAGTAATG | 22 |
| rev | | |
| pSopA- | ACCCAGGTTAGGCGCACTGTCAATAACTATG | 23 |
| fwd | | |
| pSopA- | CATAGTTATTGACAGTGCGCCTAACCTGGGT | 24 |
| rev | | |

3. Site-Directed Mutagenesis of SalI and FseI Restriction Sites in the pSMART-BAC Plasmid

To allow cloning genes or ATU cassette in the ATU2 and ATU3 simultaneously,

SalI restriction site in pSMART-BAC needed to be abrogated. Because the SalI restriction site is located in a non-coding region of the plasmid, it could be mutated without concern for in-frame amino acid sequence. The mutation changed the T6196-A. Because the FseI site of pSMART-BAC is located inside the OriV gene, we mutated G1020-C rendering 99.8% homology to the original OriV. Similarly, the site-directed mutagenesis was carried out using GeneART Site-Directed Mutagenesis PLUS kit (ThermoFisher Scientific). The primers were designed to replace nucleotide T6196-A and G1020-C(Table 2). The protocol used was the same as above with different extension times: 1 min 20 sec for TUBE1 and 2 min 35 sec for TUBE2. The mutated clones were confirmed by SalI and FseI digestions and sequencing.

The resulting plasmid was named pSMAC-BAC.

Construction of pSMAC-BAC Containing Full-Length MV Schwarz Infectious Genome

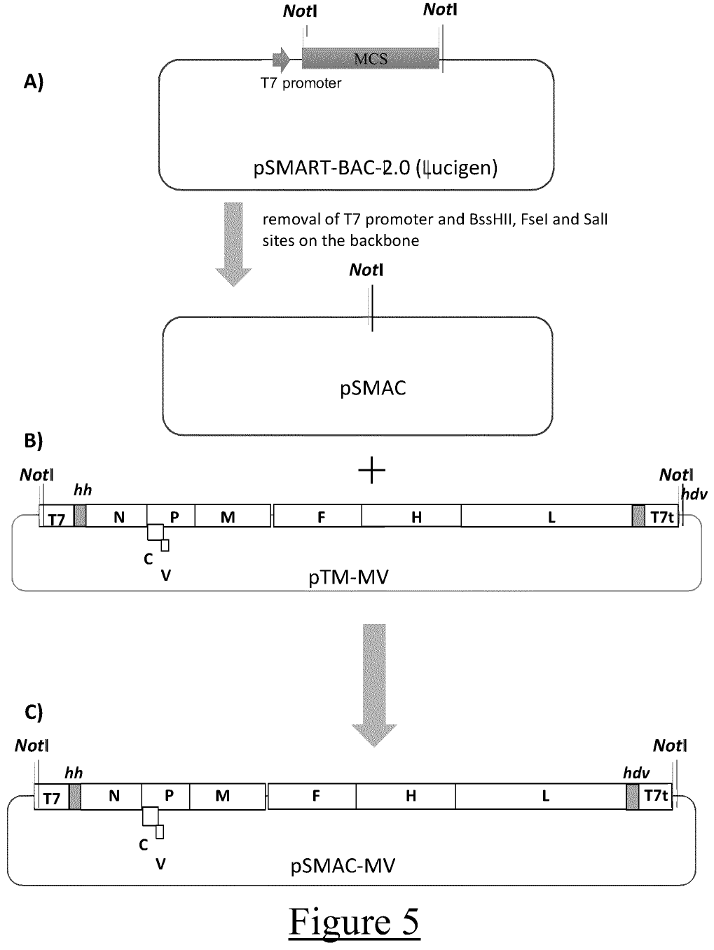

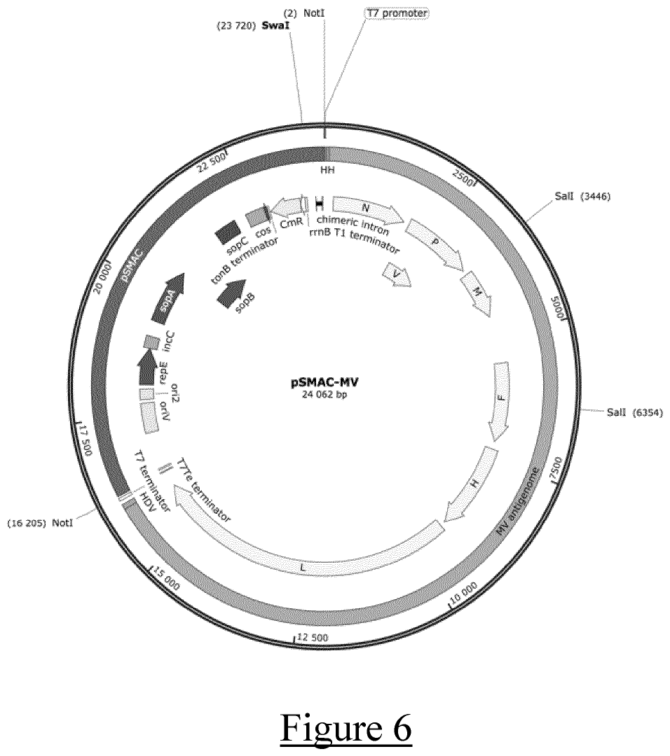

The nucleotide sequence of the full-length antigenomic (+) RNA strand of Schwarz measles virus together with T7 RNA polymerase promoter and hammerhead ribozyme sequences in 5′ and hepatitis delta ribozyme and T7 RNA polymerase terminator sequences in 3′ was inserted into the pSMAC-BAC plasmid. This sequence was simply excised from plasmid pTM-MVSchw using NotI restriction enzyme and cloned into NotI open pSMAC plasmid after alkaline phosphatase treatment to prevent self-ligation of the vector (FIG. 5). The resulting ligation reaction was used to transform NEB10 competent bacteria using manufacturer protocol. The positive clones were screened on LB/Chloramphenicol agar plates. Isolated clones were picked for plasmid amplification and purification and then sequenced to confirm the cloning. The resulting plasmid was named pSMAC-MV (FIG. 6).

Construction of pSMAC-BAC Containing Full-Length MV Infectious Genome with ATU Containing Fluorescent Proteins GFP and mCherry

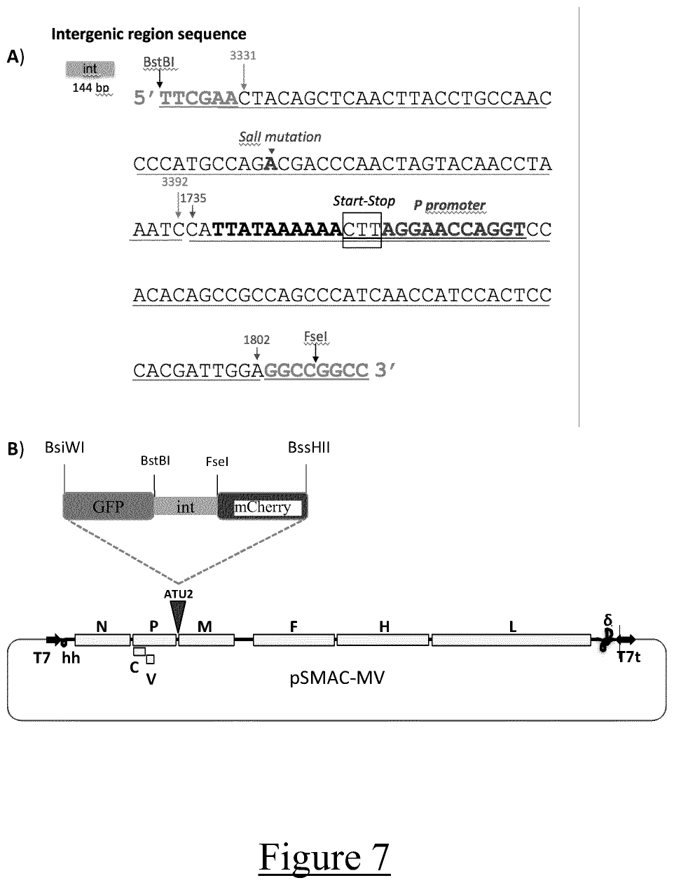

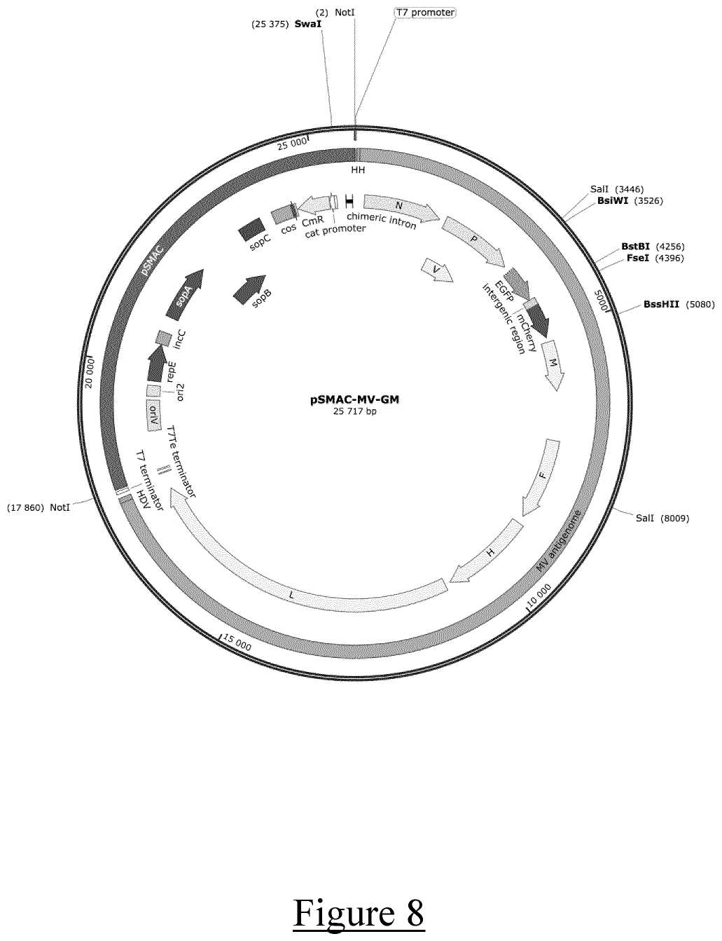

To allow an easy observation of rescued virus and comparison between pTM and pSMAC rescue efficiency, we cloned into the pSMAC-BAC plasmid a recombinant MV full-length cDNA antigenomic sequence expressing simultaneously the GFP and the mCherry fluorescent proteins in a single position from ATU2 (FIG. 7). We first generated an intergenic sequence allowing the expression of two additional sequences from a single ATU (FIG. 7A). This sequence is composed of nucleotides 3331-3392 of MV Schwarz genomic sequence with a single T to A nucleotide change at position 3365 to mutate a SalI restriction site linked to nucleotides 1735-1802 of MV Schwarz genomic sequence containing the P promoter. Two restriction sites (6-base and 8-base respectively) were added at the 5′ and 3′ end of the intergenic sequence. Unique BstBI and FseI restriction sites were added to facilitate further insertion of additional genes. The encoding sequences of fluorescent GFP and mCherry proteins were added on both sides of this intergenic sequence and the whole fragment was introduced into BsiWI and BssHII sites of pSMAC-MV ATU2 to allow the simultaneous expression of GFP and mCherry from ATU2 (FIG. 7B). The resulting plasmid was named pSMAC-MV-GM (FIG. 8).

Recovery of infectious virus from pSMAC-MV-GM and comparison with pTM-MV-GM

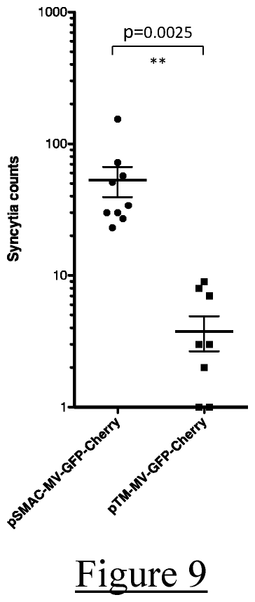

To assess the efficiency of recovery of infectious recombinant virus from pSMAC-BAC compared to pTM, we used the previously described reverse genetics method with the helper cell line HEK293-T7-MV (use of the helper-cell-based rescue system is for example described by Radecke (Radecke et al., EMBO J., 1995, 14:5773-5784) and modified by Parks (Parks et al., J. Virol., 1999, 73:3560-3566)). After co-transfection of HEK293-T7-MV cells with 5 μg of vector plasmids together with 20 ng of pEMC-L plasmid and calcium phosphate in 35 mm dishes and heat shock at 42° C. for three hours. After 48-hour incubation, transfected cells were overlaid onto monolayers of Vero cells (ATCC CCL-81) on 10 cm petri dishes. Cells were then gently covered with carboxy-methyl-cellulose (CMC 50% in DMEM and 5% FCS) to allow single clonal syncytia appearance and to avoid spreading and multiplication of clones. pTM-MV-GFP-mCherry (pTM-MV-GM) was used as a comparison control. Rescue experiments were done in triplicate and three separate biological repeats were performed. After three days fluorescent syncytia were counted. Fluorescent infectious recombinant virus was rescued 15-50 times more efficiently from pSMAC-MV-GM than from pTM-MV-GM (FIG. 9). This demonstrates that pSMAC strongly enhances the recovery of recombinant MV because the sole difference is the backbone vector between pSMAC-MV-GM and pTM-MV-GM emphasizing the advantage of pBAC in rescue MV more efficiently.

pSMAC Plasmid Accommodates Larger Additional Sequences with Multiple Antigens Constructions

The versatility of MV recombinant vaccine vector has demonstrated a solid proof of principle in humans. Clinical trials with a measles-chikungunya vaccine candidate (MV-CHIKV) have shown the safety and the immunogenicity of this vaccine platform in volunteers with preexisting immunity to measles. The use of pSMAC-MV plasmid as MV vector makes it possible to insert larger additional sequences into MV antigenomic sequence as BAC plasmids are known to stably accommodate large amounts of DNA. To prove this concept, we generated a single pSMAC-MV recombinant vector able to express simultaneously three large immunogens from three distinct pathogens: CHIKV, WNV, and ZIKV. We inserted antigens that we previously identified as protective when expressed individually from single MV vectors. The pSMAC-MV-CHIKV-WNV-ZIKV triple-antigen construct (FIG. 13) contains 7476 bp additional nucleotides including the intergenic sequences, i.e. more than 3000 bp longer than the largest recombinant MV described so far.

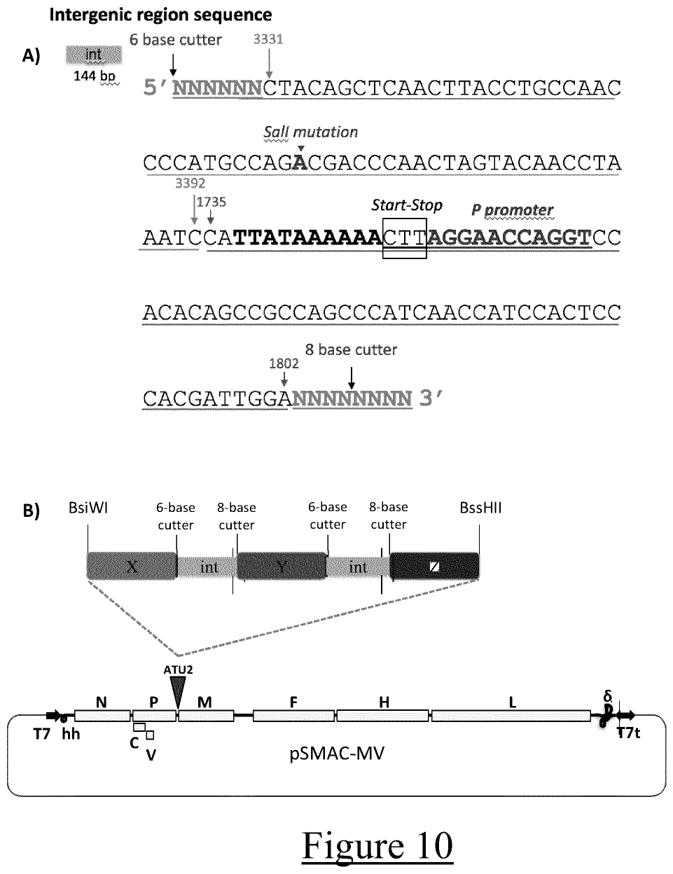

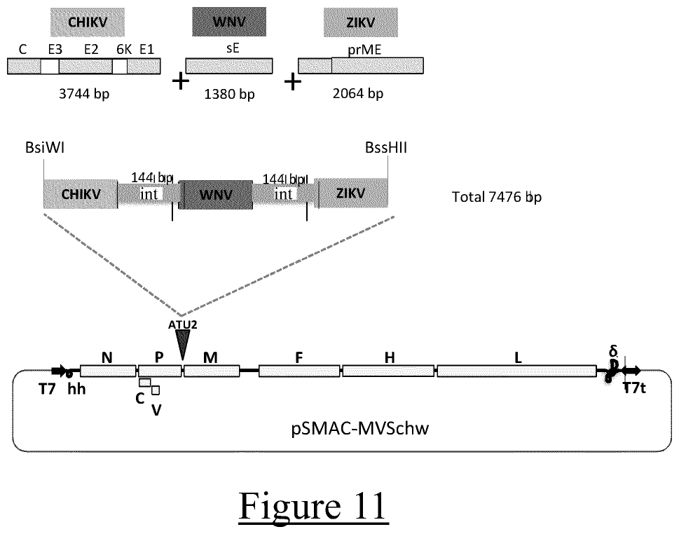

The three antigens were inserted between P and M genes of MV vector as this position allows high expression of the additional transgenes. To express the three transgenes independently from this single position of MV genome, we added between each antigenic sequence an intergenic sequence based on MV cis-acting elements allowing their expression as standard MV genes. The intergenic sequence was designed to contain the essential elements, polyA tail and measles promoter and the CTT start-stop signal for MV polymerase. This intergenic sequence is a combination of N promoter (position 1734-1784) and a copied sequence of P/M intergenic region (position 3331-3409). Several restriction sites were also added to allow easy manipulation of genes within the multiple ATUs. The detailed sequences are depicted in FIGS. 10, 11 and 12.

Construction of Triple Antigens of CHIKV, WNV, and ZIKV with MV Intergenic Region

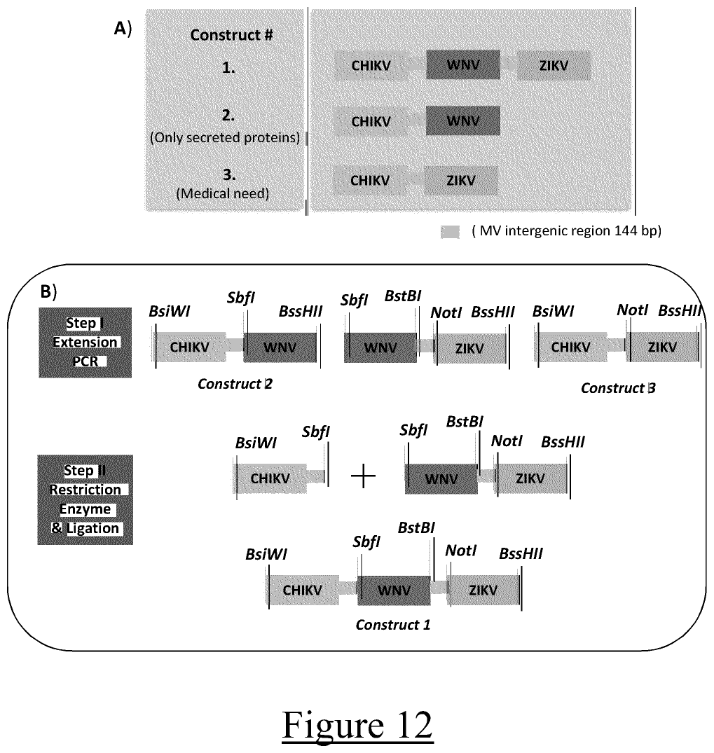

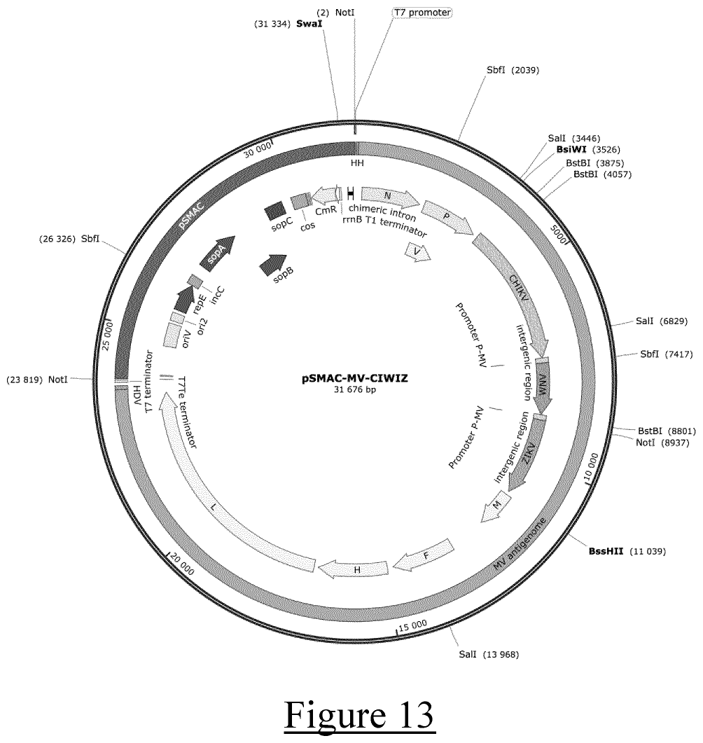

The amplification of the triple immunogens with intergenic region (FIG. 12) was performed by extension PCR using primers in Table 3. The fragment CHIKV-intergenic-WNV (CIW) was amplified in two-step extension PCR. First, CHIKV fragment was amplified using primers BsiWI-CHIKV and inter-anti-CHIKV using pTM2-CHIKV as template, while WNV fragment was amplified using primers inter-sens-Sbf1-WNV and WNV-BssHII with pTM-WNV as template. The PCR reactions were carried out using Q5 2× mastermix (NEB) protocol recommended by the manufacturer. Both PCR products were analyzed and purified for further use in the second PCR (FIG. 12). The final product of CIW was digested with BsiWI and BssHII restriction enzymes, and then ligated into the pSMAC-MV to generate pSMAC-MV-CIW with the double antigenic fragment CIW inserted into ATU2.

TABLE 3
Primers used for extension PCR in the
construction of a triple antigens (CHIKV, WNV and
ZIKV) construct, with intergenic region of MV for
individually expression of the antigens

| Primer | | SEQ |
|---|---|---|
| name | Sequence 5′-3′ | ID |
| BsiWI- | TTACGTACGATGGAGTTCATCCCAACCCAAAC | 27 |
| CHIKV | | |
| inter anti- | ATAATGGATTTAGGTTGTACTAGTTGGGTCGACT | 28 |
| CHIKV | GGCATGGGGTTGGCAGGTAAGTTGAGCTGTAGTT | |
| | CGAACTATTAGTGCCTGCTGAACGACAC | |
| inter sens- | AACCTAAATCCATTATAAAAAACTTAGGAACCAG | 29 |
| Sbfl-WNV | GTCCACACAGCCGCCAGCCCATCAACCATCCACT | |
| | CCCACGATTGGACCTGCAGGATGAGAGTTGTGTT | |
| | TGTC | |
| inter anti- | ATAATGGATTTAGGTTGTACTAGTTGGGTCGACT | 30 |
| WNV | GGCATGGGGTTGGCAGGTAAGTTGAGCTGTAGTT | |
| | CGAATTAGACAGCCTTCCCAAC | |
| inter sens- | AACCTAAACCATTATAAAAAACTTAGGAACCAGG | 31 |
| ZIKV | TCCACACAGCCGCCAGCCCATCAACCATCCACTC | |
| | CCACGATTGGAGGCCGGCCATGGAGAAGAAGCGG | |
| | AGAG | |
| WNV- | TGAGCGCGCTTAGACAGCCTTCCCAAC | 32 |
| BssHII | | |
| ZIKV- | TTAGCGCGCTCATCAGGCAGACACG | 33 |
| BssHII | | |
| Sbfl-WNV | ATTCCTGCAGGATGAGAGTTGTGTTTGTC | 34 |
| Sbfl-ZIKV | TTACCTGCAGGATGGAGAAGAAGCGGAGAG | 35 |
| BsiWI- | TTACGTACGATGGAGAAGAAGCGGAGAG | 36 |
| ZIKV | | |
| inter anti- | ATAATGGATTTAGGTTGTACTAGTTGGGTCGACT | 37 |
| ZIKV | GGCATGGGGTTGGCAGGTAAGTTGAGCTGTAGTT | |
| | CGAATCATCAGGCAGACACGGCGGTGGAC | |
| inter sens- | AACCTAAATCCATTATAAAAAACTTAGGAACCAG | 38 |
| Sbfl- | GTCCACACAGCCGCCAGCCCATCAACCATCCACT | |
| CHIKV | CCCACGATTGGACCTGCAGGATGGAGTTCATCCC | |
| | AACCCAAAC | |
| CHIKV- | TTAGCGCGCCTATTAGTGCCTGCTGAACGAC | 39 |
| BssHII | | |

The WNV-intergenic-ZIKV fragment (WIZ) was created in the same manner using the primers Sbf-WNV and inter-anti-WNV with pTM-WNV as template, while primers inter-sens-ZIKV and BssHII-ZIKV using pCDNA-ZIKV as template. The combined WIZ fragment was further digested with Sbfl and BssHII restriction enzymes. The purified WIZ fragment was then ligated to the CIW fragment similarly cut to generate pSMAC-MV-CIWIZ containing the triple antigenic fragment CIWIZ inserted into ATU2 (FIG. 12). We have also generated in the same way the bivalent vaccine candidates CHIKV-inter-ZIKV (CIZ) and ZIKV-inter-CHIKV (ZIC) in the same pSMAC-MV vector. Plasmids were fully sequenced to control integrity of cloning.

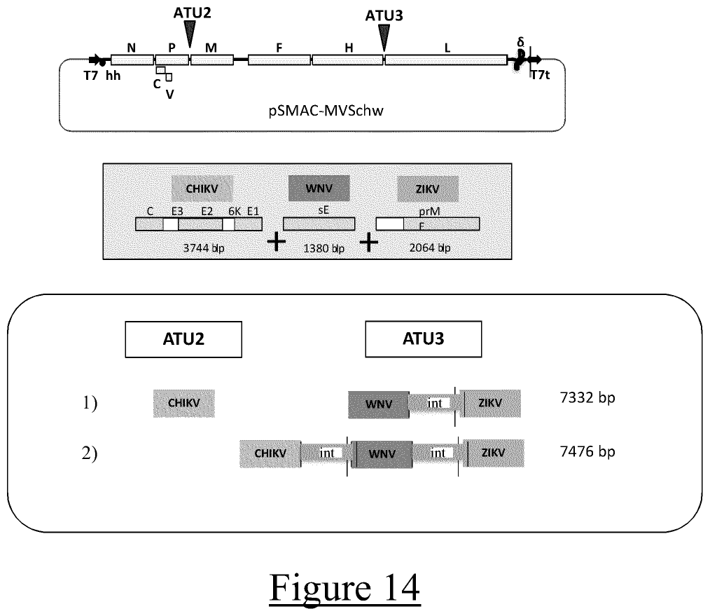

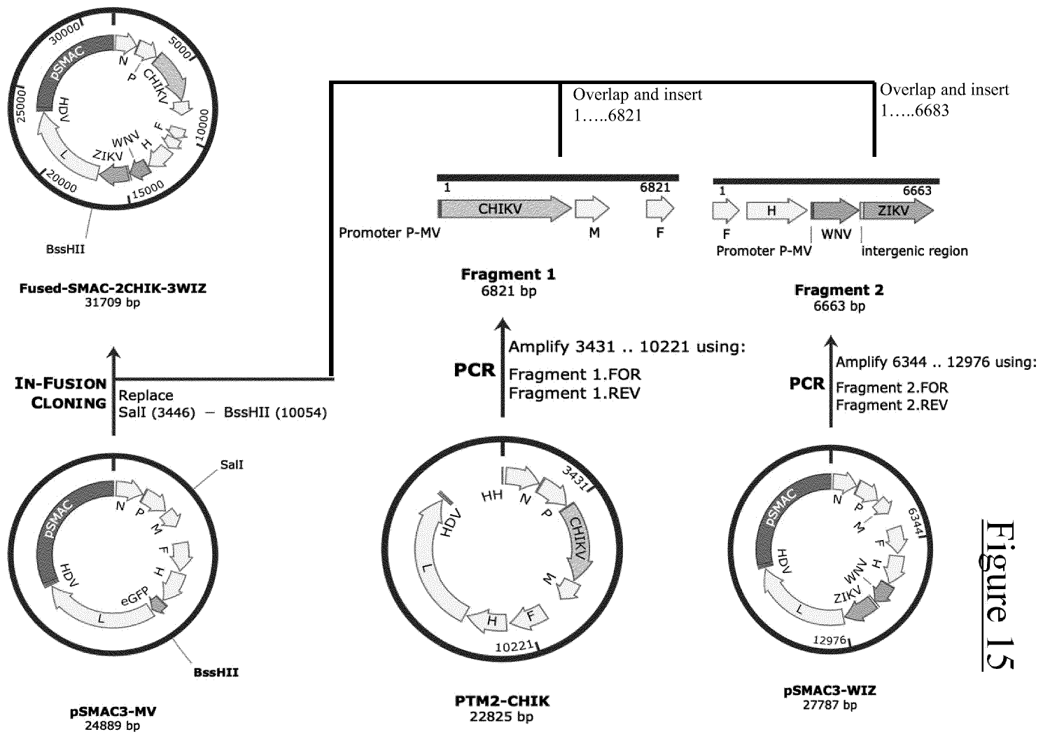

The triple antigenic fragment of CIWIZ (CHIKV-intergenic-WNV-intergenic-ZIKA) was also cloned into the ATU3 (FIG. 14). First, the complete MV genome with GFP inserted within ATU3 was cloned into pSMAC using the NotI restriction enzyme. Then the CIWIZ fragment was inserted into ATU3 in place of GFP using BsiWI and BssHII restriction sites. The resulting construct is pSMAC-MV3-CIWIZ (or pSMAC3-CIWIZ).

In order to prove that the additional genes can be expressed simultaneously from ATU2 and ATU3, the CHIKV fragment was also cloned individually into ATU2 while the WIZ (WNV-intergenic-ZIKV) fragment was cloned into ATU3 (FIG. 14). The resulting construct is pSMAC-MV-2CHIKV-3WIZ (or pSMAC-2CHIKV-3WIZ).

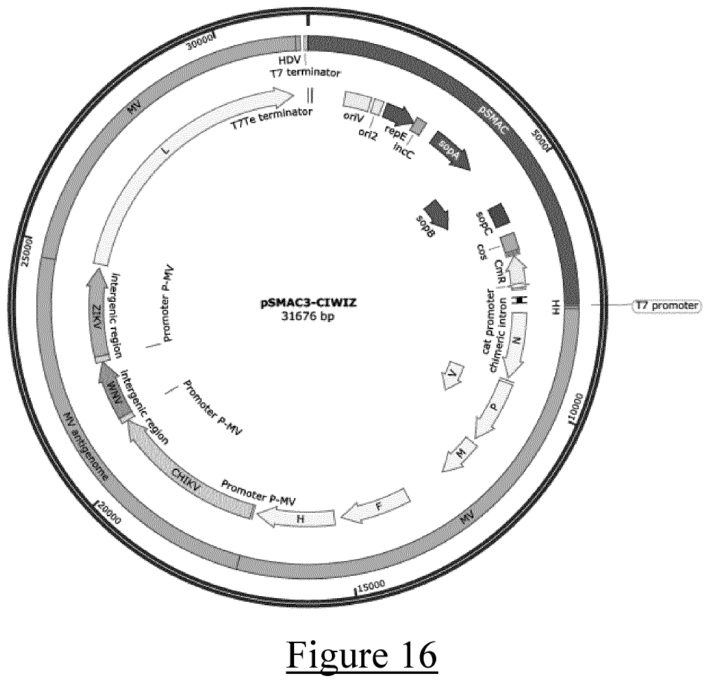

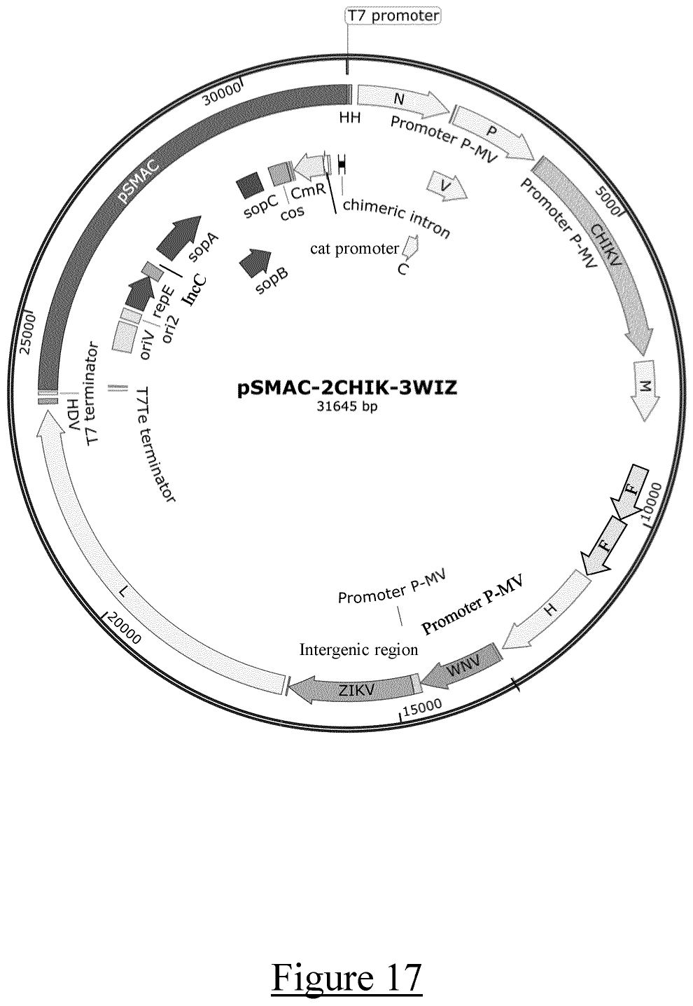

Due to limitation of restriction enzymes sites in the antigenic fragments, the In-Fusion cloning technique was used to assemble the three fragments as depicted in FIG. 15. The vector pSMAC3-MV was cut with SalI and BssHII restriction enzymes while the CHIKV fragment was amplified from pTM2-CHIKV and the WIZ fragment was amplified from pTM3-WIZ. The reaction was carried out using In-Fusion SNAP assembly (Takara) according to the manufacturer's recommendation resulting in the plasmids pSMAC-MV3-CIWIZ and pSMAC-MV-2CHIKV-3WIZ (FIGS. 16 and 17).

Rescue of Multivalent Recombinant MV

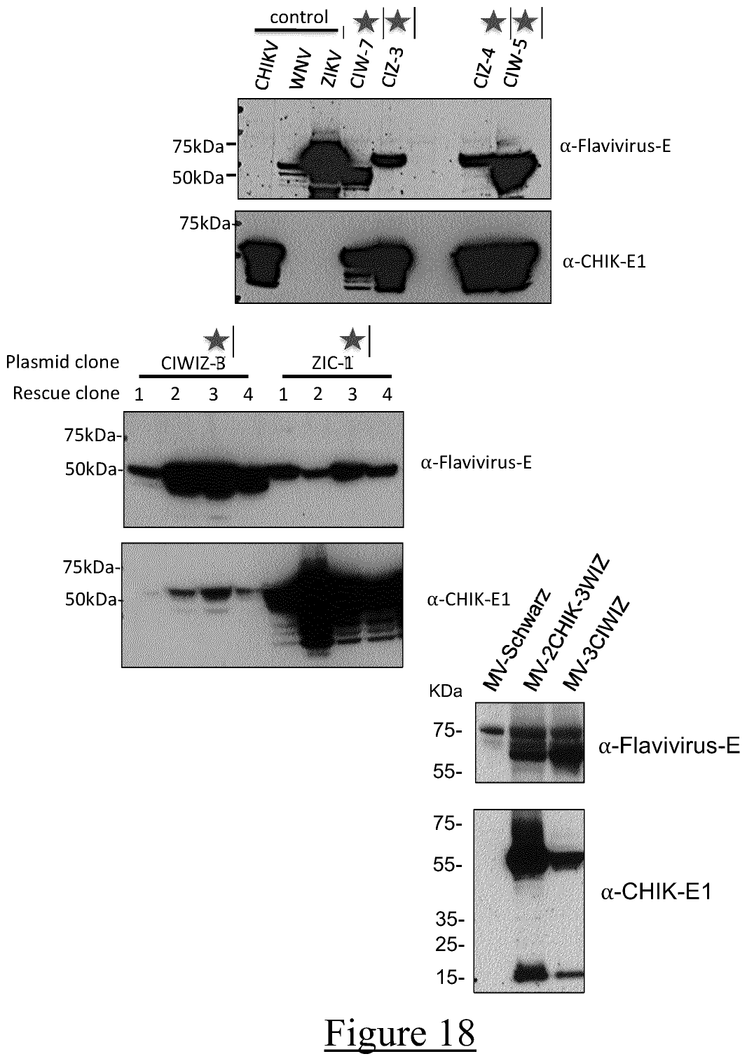

The recovery of infectious recombinant MV expressing multivalent antigens (MV-CIWIZ) from pSMAC-MV-CIWIZ, pSMAC-MV3-CIWIZ and pSMAC-MV-2CHIKV-3WIZ were performed as above³. The recombinant virus clones were picked from the coculture plates, plaque purified and amplified for stock preparation and further characterization. The targeted antigens expression was detected using western blot stained with antibodies directed to CHIKV E1 envelope protein or Flavivirus envelope (4G2) (FIG. 18).

The sequence of MV-CIWIZ rescued viruses were verified after viral RNA extraction, cDNA synthesis and RT-PCR amplification with specific primers. The sequencing result confirmed that the recombinant MV listed contains the exact sequence of the triple antigen CIWIZ.

Therefore, using pSMAC enables constructing and manipulating recombinant infectious MV carrying very large inserted additional genetic material that can still be rescued successfully.

Growth Kinetics of Multivalent Recombinant MVs.

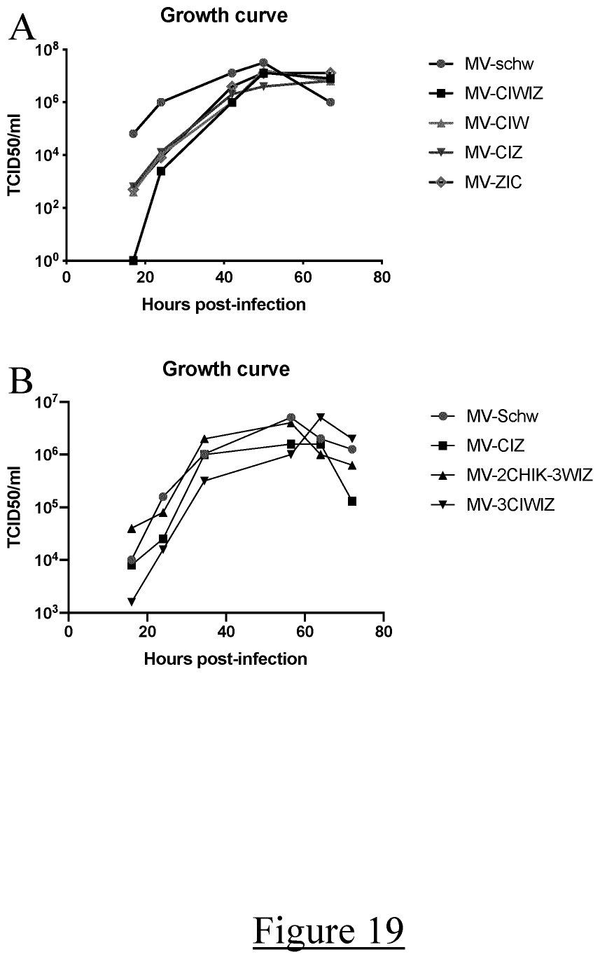

The growth kinetics of multivalent recombinant MVs was studied on Vero cells monolayers in 6-well plates. Cells were infected with the recombinant MVs at an MOI of 0.1. One plate was used per recombinant MV construct. At various time points post-infection, infected cells were scraped into 1 ml OptiMEM, lysed by freeze-thaw, clarified by centrifugation. Viral titers were determined on Vero cells seeded in 96-well plates at 7500 cells/well, and infected with serial ten-fold dilutions of virus in DMEM with 5% FBS. After incubation for 7 days, cells were stained with crystal violet, and TCID50 values were calculated and plotted according to the collected time points. The resulting growth curves of multivalent recombinant MVs are similar to MV-Schwarz, with only a slight delay in the growth of the triple CIWIZ virus resulting in similar titers achieved (FIG. 19). Accordingly, the growth kinetic of the recombinant MV constructs according to the invention is similar to the growth kinetic of the control MV-Schwarz.

Immunogenicity of Recombinant MVs Expressing Multivalent Antigens.

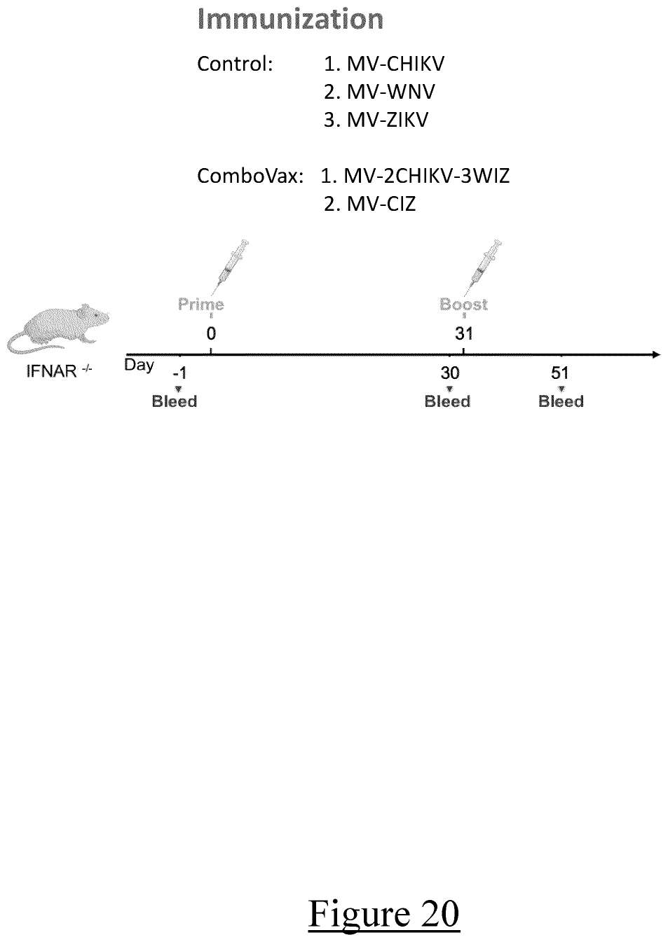

In order to verify that the multivalent antigens expressed by MV are immunogenic, Groups of 6 to 8-week-old mice deficient for type-I IFN receptor (IFNAR−/−) were intraperitoneally injected with 105 TCID50 of MV-2CHIK-3WIZ (chosen according to the highest expression of the antigens) and MV-CIZ (according to the medical interests as the CHIKV and ZIKV are endemic in the same areas) or the control single antigen recombinant MVs; MV-CHIK, MV-ZIKV and MV-WNV.[8, 14, 39]. Two immunizations were administered at a four-week interval. Sera were collected before the first immunization (day −1) and then after each immunization (day 31 and day 51) (FIG. 20). All serum samples were heat-inactivated for 30 min at 56° C. and used for ELISA to detect specific IgG to the three different antigens.

To set up the ELISA, Edmonston strain-derived MV antigens (Jena Bioscience), recombinant CHIKV E protein, WNV E protein or ZIKV E protein were coated (50 μl) on NUNC MAXISORP 96-well immuno-plates (Thermo Fisher) at 1 μg/ml in 1× phosphate-buffered saline (PBS). Coated plates were incubated overnight at 4° C., washed 3 times with washing buffer (PBS, 0.05% Tween), and further blocked for 1 h at 37° C. with blocking buffer (PBS, 0.05% Tween, 5% milk). Serum samples from immunized mice were serially diluted in the binding buffer (PBS, 0.05% Tween, 2.5% milk) and incubated on plates for 1 h at 37° C. After washing steps, an HRP-conjugated goat anti-mouse IgG (H+L) antibody (Jackson ImmunoResearch) was added for 1 h at 37° C. Antibody binding was detected by addition of the TMB substrate (Eurobio) and the reaction was stopped with 100 μl of 30% H2SO4. The optical densities were recorded at 450 and 620 nm wavelengths using the EnSpire 2300 Multilabel Plate Reader (Perkin Elmer). Endpoint titers for each individual serum sample were calculated.

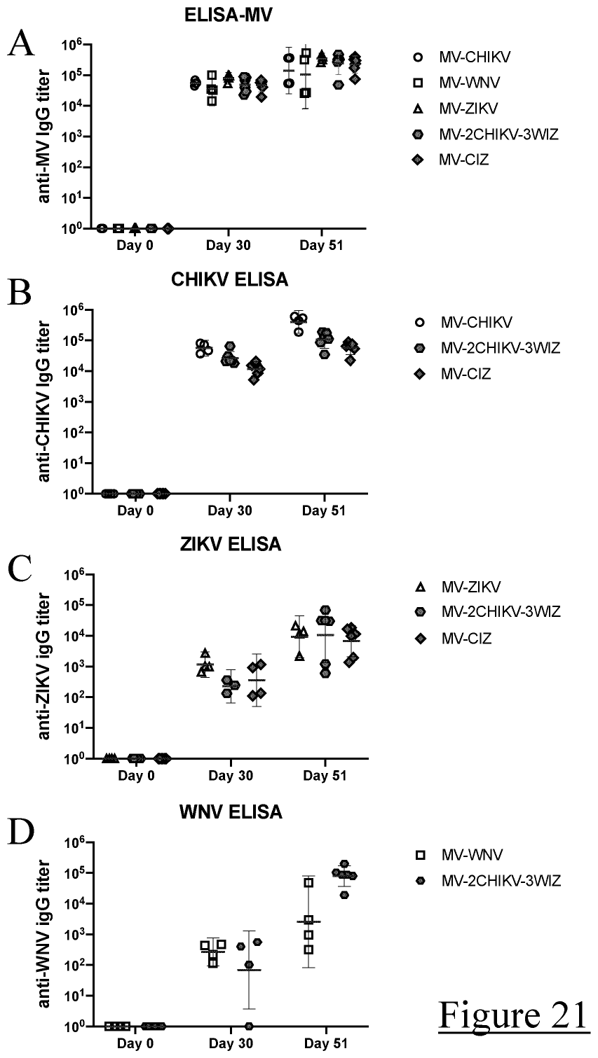

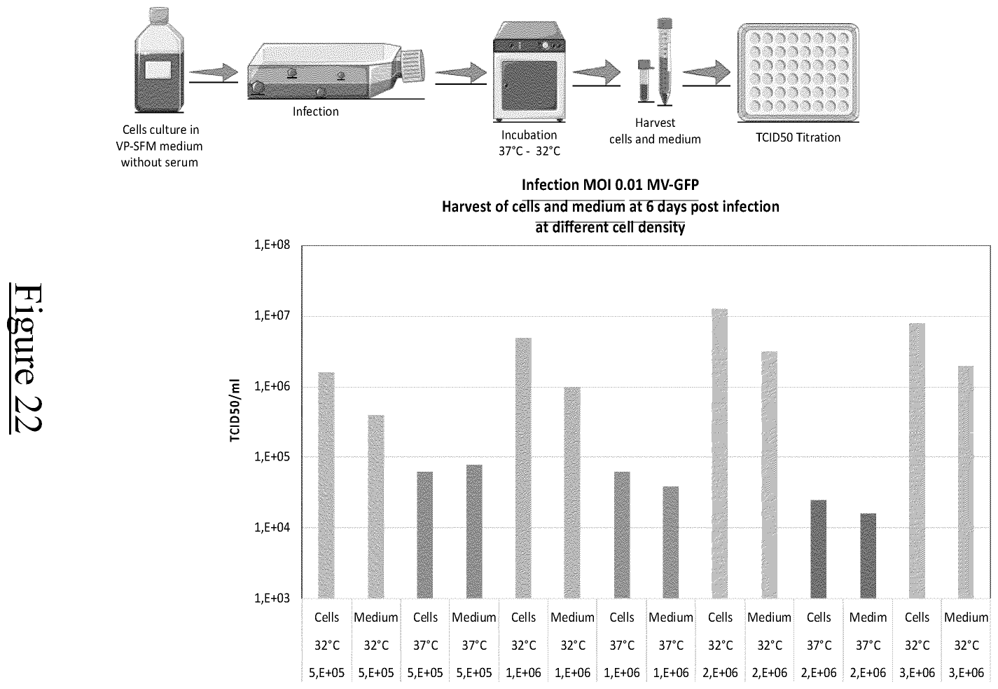

The results demonstrate that either triple or double antigens recombinant MVs stimulate specific IgG in mice at titer levels similar to those elicited by recombinant MVs expressing the corresponding single antigens (FIG. 21). These titers were previously demonstrated to be protective against challenge infection by CHIKV, WNV or ZIKV in this animal model.

This is a proof of concept that pSMAC can accommodate the cloning of approximately 7.5 kb of additional polynucleotide(s) encoding antigen(s) and allow the effective rescue of multivalent recombinant MVs with an immunogenicity comparable to single-antigen expressing recombinant MVs.

Culture of Recombinant MV on Vero Cells CCL-81 Approved for Human Use Cultures without Fetal Calf Serum

In order to prepare recombinant MV seeds ready for industrial manufacturing with no risk of contamination by BSE agents we developed a culture method to grow recombinant MV on Vero cells approved for human use and cultured in serum-free medium.

Vero CCL-81 (ATCC) cells seeded at a density of 4×10⁴ cells/cm² (3×10⁶ cells/T-75 flask) are cultured as monolayers on T-75 flasks in VP-SFM medium (Gibco/Invitrogen, ref 11681-020) supplemented with 4 mM L-glutamine (Invitrogen, ref 25030024) and Penicilin streptomycin (Invitrogen, ref 15140122) by incubation at 37° C., 5% CO2 in humid incubator. Cells are passaged after TrypLE enzyme (Invitrogen, ref 12563011) treatment for 5 minutes at room temperature. Enzyme action is stopped by dilution in 20 ml of VP-SFM medium.

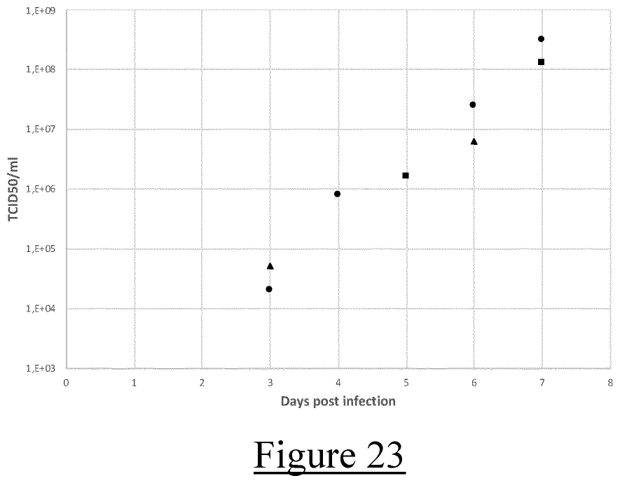

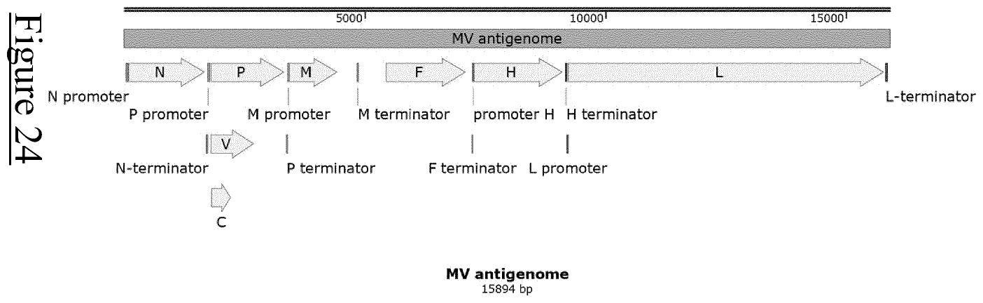

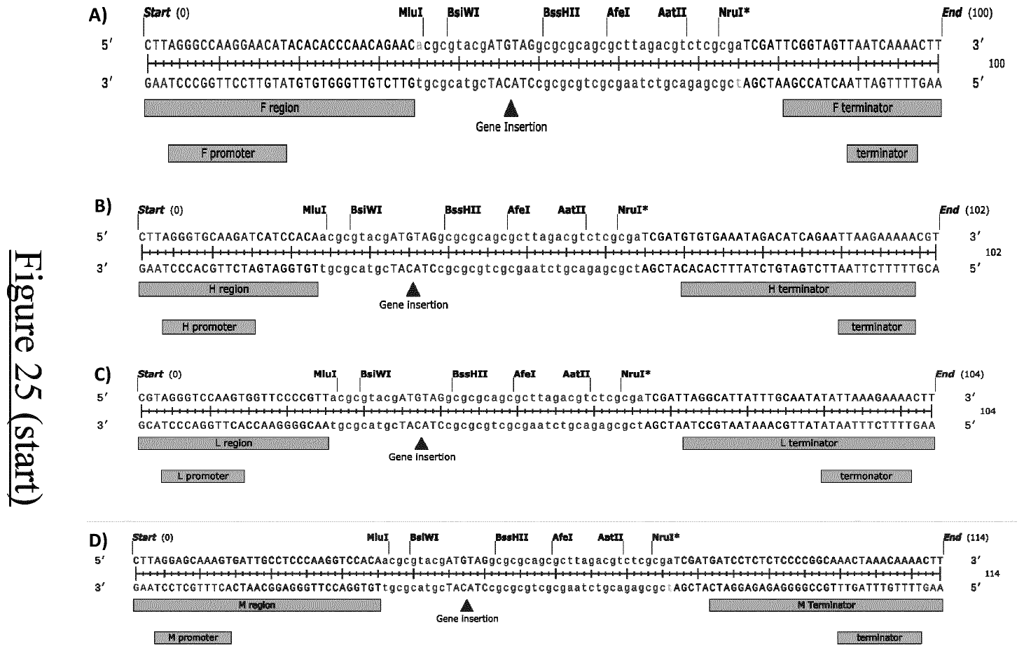

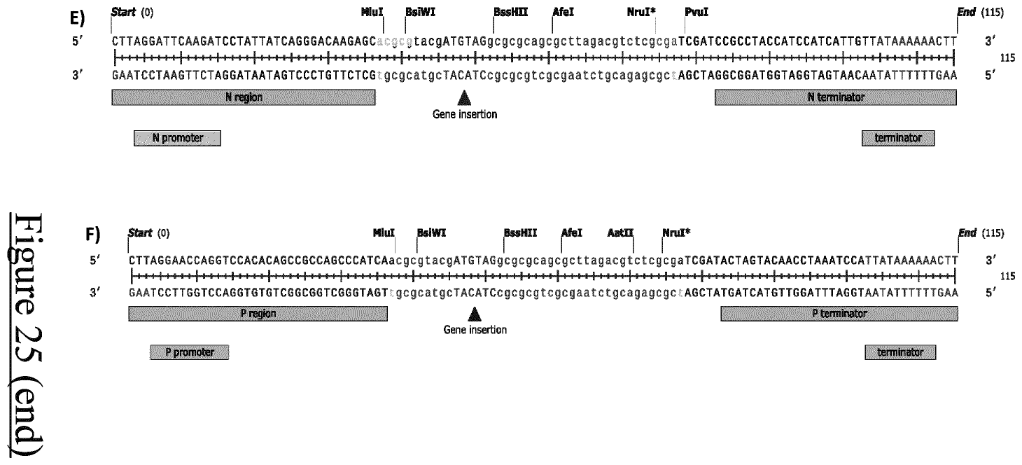

Sub-confluent cells (70-80% confluence) are washed with 10 ml of D-PBS without calcium and magnesium (Invitrogen, ref 14190094) and infected at 32 C with MV-GFP in 2 ml of VP-SFM medium at a multiplicity of infection of 0.01. After 2 hours of adsorption, the inoculum is replaced by 15-20 ml of VP-SFM medium. Culture is continued for 7 days. Medium or cells are regularly collected to determine the titer of produced virus by TCID50 titration on Vero cells (FIGS. 22 and 23). The results show that recombinant MV can be produced at titers up to 10⁸ TCID50 in medium after 7 days of production.

Protocols for the evaluation of protective immunity in mice

Any candidate vaccine, but in particular a candidate vaccine that encodes at least two antigens, preferably three antigens, originating from different species infecting human (such as the constructs pSMAC-MV3-CIWIZ and pSMAC-MV-2CHIKV-3WIZ) may be tested in mice at standard dose (104-105 TCID50/mouse) administered intraperitoneally in 6-8 week-old mice (groups of 8 mice). Control animals may receive the same dose of empty MV vector. Other groups of mice may be immunized with the same schedule with individual vaccines (MV-WNV, MV-CHIKV, MV-ZIKV). Animals may be primed at day 0 and boosted one month after. Blood samples may be collected one month after each injection for antibody analysis. ELISA specific to MV, WNV, CHIKV and ZIKV may be used. Cross reactivity of humoral response may be analyzed. To evaluate the functionality of humoral response, neutralization tests to the three viruses may be used. A vaccine candidate may lead to the neutralization of at least one virus, preferably two viruses, and most preferably the three viruses. Cell-mediated immune response may be assessed both at 7 days after priming and one month after boosting by ELISPOT assay on freshly collected splenocytes and using specific WNV, CHIKV, and ZIKV peptides.

Immunized animals may be separated in three groups to be challenged respectively by WNV, CHIKV and ZIKV. The challenges may be performed as previously published for each virus, using already determined doses. Protective efficacy will be measured by both survival and viremia analysis (RT-qPCR). A vaccine candidate may lead to immunization against one virus, two viruses or the three viruses.

Protocols for the Evaluation of Immunogenicity in Macaques

To evidence the equivalence between constructs encoding a single antigen and constructs that encodes at least two, preferably three, antigens originating from different species infecting human (such as the constructs pSMAC-MV3-CIWIZ and pSMAC-MV-2CHIKV-3WIZ), two groups of cynomolgus macaques may be immunized either with a construct vector expressing simultaneously at least 2, preferably 3, protective antigens from WNV, CHIKV and/or ZIKV, or with a mixture of the 3 independent MV vectors previously generated and expressing each of the same antigens. Ten animals may be included in both experimental groups. This number seems to be the minimum to evidence a 0.66-log difference of neutralizing activity titers with a power of 80% and an alpha risk level of 0.005.

Hematological parameters may be followed every week (blood cells counts, numeration). Blood samples may be collected for immune responses analysis at week 1, 2 and 4 after each immunization and just before boosting. Antibody titers to MV, WNV, CHIKV and ZIKV may be analyzed by using already established ELISA. Neutralizing activities may also be monitored using specific PRNT assays previously developed. A vaccine candidate may lead to the neutralization of at least one virus, preferably two viruses, and most preferably the three viruses. T cell responses may be measures by IFNg ELISPOT and intracellular cytokine staining (ICS). A vaccine candidate may lead to a T cell response against at least one virus, preferably two viruses, and most preferably the three viruses The following parameters can be analyzed: CD3, CD4, CD8, CD154, CD137, TNF-alpha, IFN-gamma, IL2. Memory response may be analyzed using the same parameters in blood samples collected at 3 months after boosting. The primary read-out may be neutralizing activity titers. These titers may be compared to the titers correlating with protection in mice challenge previously performed.

### BIBLIOGRAPHY

Lund, N., et al., The Effect of Oral Polio Vaccine at Birth on Infant Mortality: A Randomized Trial. Clin Infect Dis, 2015. 61(10): p. 1504-11.
Aaby, P. and C. S. Benn, Stopping live vaccines after disease eradication may increase mortality. Vaccine, 2020. 38(1): p. 10-14.
Combredet, C., et al., A molecularly cloned Schwarz strain of measles virus vaccine induces strong immune responses in macaques and transgenic mice. J Virol, 2003. 77(21): p. 11546-54.
Calain, P. and L. Roux, The rule of six, a basic feature for efficient replication of Sendai virus defective interfering RNA. J Virol, 1993. 67(8): p. 4822-30.
Frantz, P. N., S. Teeravechyan, and F. Tangy, Measles-derived vaccines to prevent emerging viral diseases. Microbes Infect, 2018. 20(9-10): p. 493-500.
Guerbois, M., et al., Live attenuated measles vaccine expressing HIV-1 Gag virus like particles covered with gp160DeltaV1V2 is strongly immunogenic. Virology, 2009. 388(1): p. 191-203.
Lorin, C., et al., Toxicology, biodistribution and shedding profile of a recombinant measles vaccine vector expressing HIV-1 antigens, in cynomolgus macaques. Naunyn Schmiedebergs Arch Pharmacol, 2012. 385(12): p. 1211-25.
Despres, P., et al., Live measles vaccine expressing the secreted form of the West Nile virus envelope glycoprotein protects against West Nile virus encephalitis. J Infect Dis, 2005. 191(2): p. 207-14.
Brandler, S., et al., Measles vaccine expressing the secreted form of West Nile virus envelope glycoprotein induces protective immunity in squirrel monkeys, a new model of West Nile virus infection. J Infect Dis, 2012. 206(2): p. 212-9.
1Brandler, S., et al., Pediatric measles vaccine expressing a dengue antigen induces durable serotype-specific neutralizing antibodies to dengue virus. PLoS Negl Trop Dis, 2007. 1(3): p. e96.
Brandler, S., et al., Pediatric measles vaccine expressing a dengue tetravalent antigen elicits neutralizing antibodies against all four dengue viruses. Vaccine, 2010. 28(41): p. 6730-9.
del Valle, J. R., et al., A vectored measles virus induces hepatitis B surface antigen antibodies while protecting macaques against measles virus challenge. J Virol, 2007. 81(19): p. 10597-605.
Cantarella, G., et al., Recombinant measles virus-HPV vaccine candidates for prevention of cervical carcinoma. Vaccine, 2009. 27(25-26): p. 3385-90.
Brandler, S., et al., A recombinant measles vaccine expressing chikungunya virus-like particles is strongly immunogenic and protects mice from lethal challenge with chikungunya virus. Vaccine, 2013. 31(36): p. 3718-25.
Yoneda, M., et al., Recombinant measles virus vaccine expressing the Nipah virus glycoprotein protects against lethal Nipah virus challenge. PLoS One, 2013. 8(3): p. e58414.
Yamaji, Y. and T. Nakayama, Recombinant measles viruses expressing respiratory syncytial virus proteins induced virus-specific CTL responses in cotton rats. Vaccine, 2014. 32(35): p. 4529-36.
Escriou, N., et al., Protection from SARS coronavirus conferred by live measles vaccine expressing the spike glycoprotein. Virology, 2014. 452-453: p. 32-41.
Malczyk, A. H., et al., A Highly Immunogenic and Protective Middle East Respiratory Syndrome Coronavirus Vaccine Based on a Recombinant Measles Virus Vaccine Platform. J Virol, 2015. 89(22): p. 11654-67.
Mateo, M., et al., Vaccines inducing immunity to Lassa virus glycoprotein and nucleoprotein protect macaques after a single shot. Sci Transl Med, 2019. 11(512).
Ramsauer, K., et al., Immunogenicity, safety, and tolerability of a recombinant measles-virus-based chikungunya vaccine: a randomised, double-blind, placebo-controlled, active-comparator, first-in-man trial. Lancet Infect Dis, 2015. 15(5): p. 519-27.
Reisinger, E. C., et al., Immunogenicity, safety, and tolerability of the measles-vectored chikungunya virus vaccine MV-CHIK: a double-blind, randomised, placebo-controlled and active-controlled phase 2 trial. Lancet, 2019. 392(10165): p. 2718-2727.
Domingo, E., et al., The quasispecies (extremely heterogeneous) nature of viral RNA genome populations: biological relevance—a review. Gene, 1985. 40(1): p. 1-8.
Shi, J. and D. P. Biek, A versatile low-copy-number cloning vector derived from plasmid F. Gene, 1995. 164(1): p. 55-8.
Shizuya, H. and H. Kouros-Mehr, The development and applications of the bacterial artificial chromosome cloning system. Keio J Med, 2001. 50(1): p. 26-30.
Tischer, B. K. and B. B. Kaufer, Viral bacterial artificial chromosomes: generation, mutagenesis, and removal of mini-F sequences. J Biomed Biotechnol, 2012. 2012: p. 472537.
Borenstein, R. and N. Frenkel, Cloning human herpes virus 6A genome into bacterial artificial chromosomes and study of DNA replication intermediates. Proc Natl Acad Sci USA, 2009. 106(45): p. 19138-43.
Cottingham, M. G., et al., Recombination-mediated genetic engineering of a bacterial artificial chromosome clone of modified vaccinia virus Ankara (MVA). PLoS One, 2008. 3(2): p. e1638.
Domi, A. and B. Moss, Cloning the vaccinia virus genome as a bacterial artificial chromosome in Escherichia coli and recovery of infectious virus in mammalian cells. Proc Natl Acad Sci USA, 2002. 99(19): p. 12415-20.
Meisinger-Henschel, C., et al., Introduction of the six major genomic deletions of modified vaccinia virus Ankara (MVA) into the parental vaccinia virus is not sufficient to reproduce an MVA-like phenotype in cell culture and in mice. J Virol, 2010. 84(19): p. 9907-19.
Roth, S. J., et al., Recovery of infectious virus from full-length cowpox virus (CPXV) DNA cloned as a bacterial artificial chromosome (BAC). Vet Res, 2011. 42: p. 3.
Almazan, F., et al., Engineering the largest RNA virus genome as an infectious bacterial artificial chromosome. Proc Natl Acad Sci USA, 2000. 97(10): p. 5516-21.
Almazan, F., et al., Construction of a severe acute respiratory syndrome coronavirus infectious cDNA clone and a replicon to study coronavirus RNA synthesis. J Virol, 2006. 80(21): p. 10900-6.
Fan, Z. C. and R. C. Bird, An improved reverse genetics system for generation of bovine viral diarrhea virus as a BAC cDNA. J Virol Methods, 2008. 149(2): p. 309-15.
Rasmussen, T. B., et al., Generation of recombinant pestiviruses using a full-genome amplification strategy. Vet Microbiol, 2010. 142(1-2): p. 13-7.
Jengarn, J., et al., Genetic manipulation of porcine epidemic diarrhoea virus recovered from a full-length infectious cDNA clone. J Gen Virol, 2015. 96(8): p.
Pu, S. Y., et al., Successful propagation of flavivirus infectious cDNAs by a novel method to reduce the cryptic bacterial promoter activity of virus genomes. J Virol, 2011. 85(6): p. 2927-41.
Aubry, F., et al., Flavivirus reverse genetic systems, construction techniques and applications: a historical perspective. Antiviral Res, 2015. 114: p. 67-85.
O'Connor, M., M. Peifer, and W. Bender, Construction of large DNA segments in Escherichia coli. Science, 1989. 244(4910): p. 1307-12.

1. A nucleic acid construct which comprises a cDNA molecule encoding a full length antigenomic (+) RNA strand of a non-segmented negative-sense single-stranded RNA virus, especially of a measles virus (MV), wherein the cDNA molecule further comprises inserted therein, at least a first additional transcription unit (ATU) and a second additional transcription unit (ATU), wherein the at least first and second additional transcription units (ATUs) are localized within a single intergenic region of the cDNA encoding the antigenomic (+) RNA strand as a single expression cassette, and wherein each ATU comprises a heterologous polynucleotide operably inserted within the ATU allowing the expression of a heterologous polypeptide encoded by the heterologous polynucleotide.

2. The nucleic acid construct of claim 1, wherein the non-segmented negative-sense single-stranded RNA virus is a measles virus (MV) originating from an attenuated strain, in particular an attenuated virus strain selected from the group consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, preferably the Schwarz strain, the AIK-C strain and the Zagreb strain, in particular the Schwarz strain of SEQ ID No. 62, the AIK-C strain of SEQ ID No. 58 and the Zagreb strain of SEQ ID No. 59.

3. The nucleic acid construct of claim 1, wherein the first ATU and the second ATU are localized: (i) between the P gene and the M gene of the measles virus in the cDNA molecule or (ii) between the H gene and the L gene of the measles virus in the cDNA molecule or (iii) between the N gene of the measles virus and the T7RNA polymerase promoter in the cDNA molecule, and optionally wherein the number of consecutive nucleotides in the nucleic acid construct is a multiple of six and/or wherein the number of consecutive nucleotides in the recombinant cDNA molecule is a multiple of six.

4. The nucleic acid construct according to claim 1, wherein the first ATU and/or the second ATU comprises the nucleotide sequence of SEQ ID No. 1 wherein the nucleotide sequence from position 79 to position 796 is substituted by a heterologous polynucleotide sequence encoding a heterologous polypeptide, in particular wherein said heterologous polynucleotides in the first ATU and the second ATU encode heterologous polypeptides that are different from each other and optionally wherein the first and the second heterologous polynucleotides are separated in the single intergenic region of the cDNA by a spacer sequence such as the sequence of polynucleotide of SEQ ID No. 2 or SEQ ID No. 11.

5. The nucleic acid construct according to claim 1, wherein the first ATU and/or the second ATU comprises its 5′ end towards its 3′ end of (i) the nucleotide sequence of SEQ ID No. 16 or SEQ ID No. 19 or SEQ ID No. 20 or SEQ ID No. 40 or SEQ ID No. 41 or SEQ ID No. 42 or SEQ ID No. 43 or SEQ ID No. 44 or SEQ ID No. 45, (ii) a heterologous polynucleotide sequence encoding a heterologous polypeptide, and (iii) the nucleotide sequence of SEQ ID No. 17 or SEQ ID No. 46 or SEQ ID No. 47 or SEQ ID No. 48 or SEQ ID No. 49 or SEQ ID No. 50 or SEQ ID No. 51; or
wherein the first ATU and/or the second ATU comprises the nucleotide sequence of SEQ ID No. 52, SEQ ID No. 53, SEQ ID No. 54, SEQ ID No. 55, SEQ ID No. 56, SEQ ID No. 57, wherein n corresponds to a heterologous polynucleotide sequence encoding a heterologous polypeptide, and wherein the heterologous polynucleotides present within the first ATU is different from the heterologous polynucleotide presents within the second ATU, and optionally wherein the first and the second heterologous polynucleotides are separated in the single intergenic region of the cDNA by a spacer sequence such as the sequence of polynucleotide of SEQ ID No. 2 or SEQ ID No. 11.

6. The nucleic acid construct according to claim 1, wherein a third ATU is localized within the same intergenic region where the first ATU and the second ATU are localized, the third ATU comprising a heterologous polynucleotide operably linked within the third ATU allowing the expression of a heterologous polypeptide encoded by the heterologous polynucleotide wherein said heterologous polypeptide of the third ATU is different from the heterologous polypeptides encoded by the first and the second ATUs, and wherein heterologous polynucleotides within a single intergenic region of the cDNA are separated from each other by a spacer sequence comprising or consisting of the polynucleotide of SEQ ID No. 2 or SEQ ID No. 11.

7. The nucleic acid construct according to claim 5, wherein the third ATU (i) comprises or consists in the nucleotide sequence of SEQ ID No. 1 wherein the nucleotide sequence from position 79 to position 796 is substituted by a heterologous polynucleotide sequence encoding a heterologous polypeptide; in particular the third ATU comprises of consists in the same nucleotide sequence as the first ATU or the second ATU but for the coding heterologous polynucleotide that is contains, and (ii) wherein the heterologous polynucleotide of the third ATU is separated from another heterologous polynucleotide inserted within the closest ATU in the single intergenic region of the cDNA by the polynucleotide of sequence SEQ ID No. 2 or SEQ ID No. 11.

8. The nucleic acid construct according to claim 1, wherein each of the first ATU and the second ATU and when present the third ATU comprises a heterologous polynucleotide that encodes a heterologous polypeptide that is different from the heterologous polypeptide encoded by the other ATUs and optionally wherein the heterologous polynucleotide or at least one of the ATUs, in particular of at least two ATUs or of all the ATUs, encodes multiple heterologous polypeptides.

9. The nucleic acid construct according to claim 1, wherein the heterologous polypeptides encoded by the heterologous polynucleotides inserted within the ATUs are immunogenic polypeptides originating from or derived from at least one pathogen infecting human, in particular from at least one virus infecting human, in particular the Chikungunya virus, the West Nile virus, the Zika virus, the SARS virus, the coronavirus, and/or the HIV, in particular a heterologous polypeptide comprising the C, E2 and E1 proteins of the Chikungunya virus, the sE protein of the West Nile virus, the prME protein of the Zika virus, the S and M proteins of the SARS virus and/or the S and M proteins of the coronavirus, in particular the S and M proteins of the SARS-CoV-2, and optionally wherein the heterologous polynucleotides originate or derive from different virus types.

10. The nucleic acid construct according to claim 1, wherein the intergenic region localized between the P gene and the M gene of the measles virus comprises the nucleotide sequence of SEQ ID No. 4.

11. A recombinant bacterial artificial chromosome (BAC) plasmid wherein the nucleic acid construct according to claim 1 is operably cloned.

12. The recombinant BAC plasmid according to claim 11, wherein the BAC nucleotide sequence is devoid of (i) a T7RNA polymerase promoter and/or of a CMV promoter and/or (ii) cloning sites that are present in the ATU sequences.

13. The recombinant BAC plasmid according to claim 11, wherein the bacterial artificial chromosome plasmid is selected from the group consisting of the pSMART BAC plasmid and its derivatives, pEZ-BAC plasmid, pBeloBAC11 plasmid, pBACe3.6, pBAC/OriV, pBAC-RT, pHA1, pHA2, pTARBAC2 and its derivatives, and the pBAC contruct inserted in Transmissible gastroenteritis coronavirus (TGEV), in particular is a pSMAC BAC, pEZ-BAC and pBeloBAC11 plasmid, in particular is the pSMAC-BAC plasmid, in particular comprises the polynucleotide of SEQ ID No. 3.

14. The recombinant BAC plasmid according to claim 1, wherein the promoter sequences localized on the recombinant BAC plasmid are different from the promoter sequences localized on the cDNA molecule.

15. The nucleic acid construct according to claim 1, wherein the first ATU and the second ATU comprise or consist of different polynucleotides that are selected among polynucleotides encoding heterologous polypeptides comprising or consisting of amino acid sequences of to SEQ ID No. 13 and SEQ ID No. 14, or selected among polynucleotides encoding heterologous polypeptides comprising or consisting of amino acid sequences of SEQ ID No. 13 and SEQ ID No. 15, and wherein the first heterologous polynucleotide and the second heterologous polynucleotide are separated by a spacer polynucleotide comprising or consisting in the nucleotide sequence of SEQ ID No. 2 or SEQ ID No.11.

16. The nucleic acid construct according to claim 1 and comprising a third ATU, wherein the first ATU, the second ATU and the third ATU comprise(s) or consist(s) of different polynucleotides encoding heterologous polypeptides comprising or consisting of amino acid sequences that are selected among SEQ ID No. 13, SEQ ID No. 14 and SEQ ID No. 15, and wherein the heterologous polynucleotides are separated by a spacer polynucleotide comprising or consisting in the nucleotide sequence of SEQ ID No. 2 or SEQ ID No. 11.

17. The nucleic acid construct according to claim 1, wherein the intergenic region localized between the P gene and the M gene of the measles virus in the cDNA molecule comprises all the ATUs, and wherein the heterologous polynucleotides inserted in the intergenic region are separated by a spacer polynucleotide comprising or consisting of SEQ ID No. 2 or SEQ ID No. 11.

18. A recombinant infectious non-segmented negative-sense single-stranded RNA virus, especially a recombinant infectious Measles virus, wherein the genome of said recombinant infectious virus comprises a nucleic acid construct according to claim 1.

19. A process for rescuing recombinant infectious non-segmented negative-sense single-stranded RNA virus, especially recombinant infectious measles virus expressing at least one heterologous polypeptide expressed from a heterologous nucleotide sequence inserted within an ATU localized within a cDNA molecule encoding a full length antigenomic (+) RNA strand of a measles virus (MV); comprising:
(a) In cells, in particular HEK293 helper cells, stably expressing T7 RNA polymerase and measles virus N and P proteins and/or transfected with an expression vector encoding T7 RNA polymerase and/or transfected with an expression vector encoding measles virus N and P proteins, transfecting the nucleic acid construct according to claim 1;
(b) Maintaining the transfected cells of step (a) in conditions suitable for the production of recombinant infectious virus and/or virus like particles;
(c) Infecting cells, in particular Vero cells, in conditions enabling the propagation of the recombinant infectious virus by co-cultivating these cells with the cells issued from step (b); in particular at 32° C.;
(d) Harvesting the recombinant infectious virus and/or virus like particles, in particular at 32° C.

20. A seed preparation or a stock preparation of a recombinant infectious non-segmented negative-sense single-stranded RNA virus, especially of a recombinant infectious measles virus that consists of a single virus clone or respectively an amplified single virus clone of a recombinant infectious virus rescued by reverse genetics from the nucleic acid construct according to claim 1.

21. A vaccine which comprises a clonally selected and amplified recombinant infectious negative-sense single-stranded RNA virus, clonally selected and amplified recombinant infectious measles virus obtainable from the rescue by reverse genetics of the nucleic acid construct according to claim 1.

22. A vaccine which enables stable expression of multiple heterologous polypeptides from the nucleic acid construct according to claim 1, wherein such stable expression is characterized by being maintained over at least 12 passages of the rescued recombinant infectious negative-sense single-stranded RNA virus when amplified on Vero cells, especially of the amplified recombinant infectious measles virus.

23. A combination vaccine which comprises a recombinant measles virus which encodes at least two immunogenic polypeptides originating from or derived from distinct pathogens infecting human, in particular from at least two viruses selected from the group of Chikungunya virus, West Nile virus and Zika virus, wherein the immunogenic polypeptides are expressed as individual polypeptides by the recombinant virus.

24. (canceled)

25. Ribonucleoprotein of a recombinant measles virus strain which comprises a RNA genome assembled with the transcriptase complex proteins of the measles virus wherein the genome contains the recombinant full length antigenomic (+) RNA strand of the measles virus (MV) and inserted therein, sequences encoding at least a first additional transcription unit (ATU) and a second additional transcription unit (ATU), wherein the at least first and second additional transcription units (ATUs) are localized within a single intergenic region of the recombinant antigenomic (+) RNA strand as a single expression cassette, and wherein each ATU comprises a heterologous polynucleotide operably inserted within the ATU allowing the expression of a heterologous polypeptide encoded by the heterologous polynucleotide as disclosed in claim 1.
